(19) United States
(12) Patent Application Publication
(10) Pub. No.: US 2017/0216434 A1
(43) Pub. Date: Aug. 3, 2017

(54) COMPOSITIONS AND METHODS FOR TREATMENT OF SICKLE CELL DISEASE

(71) Applicants: Wayne State University, Detroit, MI (US); Biogen, Inc., Cambridge, MA (US)

(72) Inventors: Patrick C. Hines, Grosse Pointe Park, MI (US); Moira M. Lancelot, Marshfield, WI (US); Jennell C. Jackson, Detroit, MI (US); William E. Hobbs, II, Cambridge, MA (US); Sriram Krishnamoorthy, Somerville, MA (US); David R. Light, Cambridge, MA (US)

(21) Appl. No.: 15/501,731

(22) PCT Filed: Aug. 5, 2015
(86) PCT No.: PCT/US2015/043770

§ 371 Date: Feb. 3, 2017

Related U.S. Application Data
(60) Provisional application No. 62/043,629, filed on Aug. 29, 2014.
(60) Provisional application No. 62/033,436, filed on Aug. 5, 2014.

(51) Int. Cl. A61K 39/395; G01N 33/564; A61K 31/17

(52) U.S. Cl. CPC A61K 39/39541; A61K 31/17; G01N 33/564; A61K 2300/00

(57) ABSTRACT

This invention relates to methods of treating sickle cell disease with VLA-4 antagonists and methods for evaluating the responsiveness of patients having sickle cell disease to treatment with VLA-4 antagonists.

### FIELD OF THE INVENTION

This invention relates generally to methods of treating sickle cell disease in a subject and evaluating a sample comprising blood cells of a subject. According to specific aspects, this invention relates to treatment of a subject having sickle cell disease with one or more VLA-4 antagonists and methods for evaluating the responsiveness of patients having sickle cell disease to treatment with one or more VLA-4 antagonists.

### BACKGROUND OF THE INVENTION

Sickle cell disease (SCD) is a congenital disease caused by the inheritance of a mutant β-globin allele resulting in abnormal hemoglobin, which is the oxygen carrying molecule in red blood cells (RBCs). SCD is associated with high early mortality, and it is estimated that only 35% to 50% of individuals with SCD in the US survive to age 45 (Hassell K L Am J Prey Med 38(4 Suppl):S512-21, 2010; Platt OS et al. N Engl J Med 330(23):1639-44, 1994). In the US, SCD is estimated to affect approximately 100,000 individuals, most from the African-American community (Hassell, supra).

### SUMMARY OF THE INVENTION

The invention relates, inter alia, to methods of treating sickle cell disease (SCD), methods of reducing the frequency and/or severity of acute vaso-occlusive events, and methods of treating acute vaso-occlusive events using a VLA-4 antagonist such as natalizumab. It was discovered that VLA-4 antagonists such as natalizumab can effectively reduce the symptoms of SCD. For example, the administration of a VLA-4 antagonist such as natalizumab to blood from a subject with SCD effectively inhibits adhesion of reticulocytes, immature red blood cells (RBCs) that are found in increased numbers in the peripheral blood of SCD patients, to VCAM-1, which is expressed on the endothelium. Also disclosed herein are methods for the identification subjects for treatment with a VLA-4 antagonist such as natalizumab.

Accordingly, in one aspect, the disclosure features a method of treating a subject suffering from or susceptible to sickle cell disease (SCD), the method comprising: administering to the subject a therapeutically effective amount of a composition comprising a VLA-4 antagonist, wherein the composition is administered such that one or more symptoms of SCD is prevented or reduced. In certain embodiments, the composition is administered such that the number, frequency, and/or duration of vaso-occlusive events in the subject is reduced, e.g., as compared to the number, frequency and/or frequency of vaso-occlusive events in the subject prior to treatment. In certain embodiments, the composition is administered such that red blood cell survival is increased in the subject, e.g., as compared to the red blood cell survival in the subject prior to treatment. In certain embodiments, the composition is administered such that hemoglobin levels are increased in the subject, e.g., as compared to the hemoglobin levels in the subject prior to treatment.

In another aspect, the disclosure features methods of treating an acute vaso-occlusive event in a subject, the method comprising: administering to the subject a therapeutically effective amount of a composition comprising a VLA-4 antagonist, wherein the composition is administered such that the severity and/or frequency of the acute vaso-occlusive event is reduced in the subject. In some embodiments, the composition is administered to the subject within 7 days, 6 days, 5 days, 4 days, 3 days, 2 days, or 1 day after the onset of the vaso-occlusive event in the subject.

In another aspect, the disclosure features methods of reducing the frequency of an acute vaso-occlusive event in a subject, the method comprising: administering to the subject a therapeutically effective amount of a composition comprising a VLA-4 antagonist, wherein the composition is administered such that the frequency of the acute vaso-occlusive event is reduced in the subject.

In certain embodiments, the therapeutically effective amount of the composition is less than 300 mg, e.g., 100-200 mg, e.g., 150 mg. In some embodiments, the therapeutically effective amount of the composition is 200-400 mg, e.g., 300 mg. In some embodiments, the therapeutically effective amount of the composition is greater than 300 mg, e.g., 400-500 mg, e.g., 450 mg. In some embodiments, the composition is administered by intravenous administration. In one embodiment, the composition is administered once a week, once every two weeks, once every three weeks or monthly, e.g., once every four weeks.

In certain embodiments, the therapeutically effective amount of the composition is 50-100 mg, e.g.,75 mg. In some embodiments, the composition is administered subcutaneously. In one embodiment, the composition is administered once a week, once every two weeks, once every three weeks or monthly, e.g., once every four weeks.

In certain embodiments, the α4 antagonist is an anti-VLA-4 antibody molecule, e.g., an anti-VLA-4 antibody molecule described herein. In particular embodiments, the anti-VLA-4 antibody molecule is a monoclonal, a humanized, a human, a chimeric anti-VLA-4 antibody molecule. In some embodiments, the VLA-4 antagonist is an α4-binding fragment of an anti-VLA-4 antibody. In certain embodiments, the α4 binding fragment is an Fab, Fab′, F(ab′)2, or Fv fragment.

In some embodiments, the anti-VLA-4 antibody molecule comprises one or more, preferably all, of HC CDR1, HC CDR2, HC CDR3, LC CDR1, LC CDR2 and LC CDR3 of natalizumab.

In certain embodiments, the VLA-4 antagonist is administered as a monotherapy. In particular embodiments, the VLA-4 antagonist is not administered in combination with hydroxyurea.

In some embodiments, the VLA-4 antagonist is administered in combination with an additional agent or procedure. In certain embodiments, the additional agent is a chemotherapeutic agent, e.g, hydroxyurea, e.g., administered to the subject in a dose of between 10 and 40 mg/kg/day. In particular embodiments, the additional agent is an analgesic, e.g., an opiod analgesic. In some embodiments, the additional procedure is a transfusion, e.g., a red blood cell transfusion or a transplant, e.g., a hematopoietic stem cell transplant (HSCT). In some embodiments, the VLA-4 antagonist and the additional agent or procedure are administered simultaneously to the subject. In particular embodiments, the VLA-4 antagonist and the additional agent or procedure are administered sequentially to the subject.

In some embodiments, the subject has not received a previous treatment with a VLA-4 antagonist, e.g., natalizumab. In some embodiments, the subject does not have or is not at risk for developing progressive multifocal leukoencephalopathy (PML).

In some embodiments, the subject has greater than 2%, e.g., greater than 5%, e.g., greater than 10%, or more, reticulocytes in their blood.

In some embodiments, the subject has 70 g/dL or more, e.g., 80 g/dL or more, hemoglobin in their blood before administration. In some embodiments, administration is temporarily discontinued if the subject has less than 67 g/dL hemoglobin in their blood. In particular embodiments, administration is permanently discontinued if the subject has less than 55 g/dL hemoglobin in their blood. In certain embodiments, administration is permanently if hemoglobin levels in the subject's blood decrease by more than 25 g/L over a 1 week period.

In certain embodiments, the subject is an adult human subject. In some embodiments, the subject is a pediatric human subject, e.g., 18 years or younger.

In another aspect, the disclosure features methods evaluating a sample comprising blood cells from a subject, the method comprising: (a) subjecting a first sample comprising blood cells that has been isolated from the subject to a flow adhesion assay through a channel, e.g., by perfusion via one or more microfluidic channels, wherein the channel is coated with VCAM-1 and wherein the flow adhesion assay is performed under shear stress conditions; (b) determining a level of adhesion of blood cells from the first sample to the channel; (c) contacting a second sample comprising blood cells that has been isolated from the subject with a VLA-4 antagonist, e.g., a VLA-4 binding antibody described herein; (d) subjecting the second sample to flow adhesion assay through a channel, e.g., by perfusion via one or more microfluidic channels, wherein the channel is coated with VCAM-1 and wherein the flow adhesion assay is performed under shear stress conditions; (e) determining a level of adhesion of blood cells from the second sample to the channel, e.g., microfluidic channel; and (f) identifying the subject as a candidate for treatment with a VLA-4 antagonist, e.g., a VLA-4 binding antibody described herein, if the level of adhesion determined in (e) is less than the level of adhesion determined in (b). In some embodiments, the method further comprises a step of obtaining the sample comprising blood cells from the subject. In certain embodiments, the method further comprises a step of administering a VLA-4 antagonist, e.g., a VLA-4 binding antibody described herein, to the subject. In some embodiments, the blood cells are red blood cells (RBCs). In particular embodiments, the blood cells are reticulocytes. In some embodiments the subject is selected for treatment with the VLA-4 antagonist based upon an evaluation of any of the methods described herein.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description, and from the claims.

### BRIEF DESCRIPTION OF THE DRAWINGS

FIGS. 1A, 1B, and 1C show representative whole blood flow cytometry (FCM) analysis of a SCD donor blood sample. Reticulocytes were stained with thiazole orange. FIG. 1A is a FCM dot plot showing RBC in gate P1 and platelets in gate P2. In FIG. 1B, RBCs were identified based on surface staining for CD235a. In FIG. 1C, platelets from gate P2 show weak or no staining for CD235a.

FIGS. 2A, 2B, 2C, and 2D show representative whole blood flow cytometry (FCM) analysis of healthy donor blood sample. In FIGS. 2A and 2C (top dot plot and bottom histogram) reticulocytes are unstained. In FIGS. 2B and 2D (top dot plot and bottom histogram) reticulocytes are stained with thiazole orange. Note the relatively low level of staining of reticulocytes in the healthy subject in accordance with published reports of reticulocyte composition in whole blood in healthy donors, which varies from 0.5-1.5%.

FIGS. 3A, 3B, 3C, and 3D show representative whole blood flow cytometry (FCM) analysis of a SCD donor blood sample. In FIGS. 3A and 3C (top dot plot and bottom histogram) reticulocytes are unstained. In FIGS. 3B and 3D (top dot plot and bottom histogram) reticulocytes are stained with thiazole orange. Note the higher occurrence of reticulocytes in peripheral blood from SCD subject (B) where it ranges anywhere from 2-45% (Swerlick 1993).

FIGS. 4A, 4B, and 4C show natalizumab staining of SCD reticulocytes and leukocytes. In FIG. 4A, thiazole orange stained cells were gated as reticulocytes. FIG. 4B is a dot plot representation and P10 gate represents thiazole orange positive cells that are stained for surface VLA-4 using control IgG4. FIG. 4C is a histogram representation of the MFIs of VLA-4 staining of reticulocytes in the P1 gate of FIG. 4A.

FIGS. 5A, 5B, and 5C show natalizumab staining of SCD reticulocytes and leukocytes. FIG. 5A is flow cytometry analysis of whole blood stained with natalizumab (10 μg/mL) and reticulocyte stain (thiazole orange). Thiazole orange stained cells were gated as reticulocytes. FIG. 5B is a dot plot representation and P1 gate represents thiazole orange positive cells that are stained for surface VLA-4. FIG. 5C is a histogram representation of the MFIs of VLA-4 staining of reticulocytes in the P1 gate of FIG. 5A.

FIGS. 6A, 6B, and 6C show natalizumab staining of SCD reticulocytes and leukocytes. In FIG. 6A mononuclear leukocytes were gated based on CD45 staining and side scatter, shown in gate P1. In FIGS. 6B and 6C, gated mononuclear cells were tested for natalizumab binding (6B) versus IgG4 (6C).

FIG. 7 shows a comparison of natalizumab and anti-CD29 antibody binding to reticulocytes. VLA-4 staining was carried out as described in Experiment 1 using either natalizumab (binds to the VLA-4 integrin α4-subunit) or anti-CD29 antibody (binds to the p1-subunit) and compared to IgG4 or isotype (iso) matched IgG1 antibody controls, respectively.

FIGS. 8A, 8B, and 8C show natalizumab dose-dependent binding to SCD leukocytes and reticulocytes. Whole blood was incubated with increasing concentrations of natalizumab or IgG4. Mononuclear leukocytes were gated using CD45 staining and side scatter properties. Reticulocytes were gated by thiazole orange staining Saturation curves were determined in whole blood samples from adult SCD donors. SCD leukocytes (FIG. 8A), SCD reticulocytes (FIG. 8B), and healthy donor leukocytes (FIG. 8C) are shown. Natalizumab binding to reticulocytes from healthy donor blood was below the limit of detection. Results shown are the mean±SD from 9 SCD donor samples (FIG. 8A), 8 SCD donor samples (FIG. 8B), and 4 healthy donor samples (FIG. 8C).

FIGS. 9A and 9B show natalizumab inhibition of whole blood and isolated leukocyte adhesion to VCAM-1 in SCD blood (pediatric SCD patient donors). Whole blood (FIG. 9A) and isolated leukocytes (FIG. 9B) from pediatric SCD patient donors were subjected to adhesion to VCAM-1 in microfluidic chambers under physiological conditions of shear stress (1 dynes/cm2; pulsatile flow at 1.67 Hz), in the presence of increasing concentrations of natalizumab or IgG4 (mcg/mL=μg/mL). Results are shown as percent inhibition of adhesion by each of the treatments and include data from 9 to 10 pediatric SCD donors at each dose.

FIGS. 10A, 10B, 10C, and 10D show natalizumab inhibiting whole blood and isolated leukocyte adhesion to VCAM-1 in SCD blood under physiological flow (adult SCD donors). In an adhesion assay, at concentrations above 0.1 to 10.0 μg/mL, natalizumab effectively blocked binding of total SCD whole blood cells (FIG. 10A), of the SCD leukocytes in the whole blood sample (FIG. 10B), the SCD reticuloctyes in the whole blood sample (FIG. 10C), and isolated leukocytes from these donor samples (FIG. 10D) as shown by fixing and staining the adherent cells for appropriate markers. Results are shown as the percent inhibition of blood cell adhesion and include data from 7 adult SCD donor samples.

FIGS. 11A and 11B show effect of natalizumab on of adhesion of leukocytes and reticulocytes. Natalizumab binding to SCD leukocytes or reticulocytes was compared to inhibition of cell adhesion to VCAM-1 under physiological flow for both whole blood assays. Assay results compared at 0.001 (black), 0.01 (purple), 0.1 (red), 1.0 (green) and 10 (blue) μg/mL. Correlation is nonlinear and increased percent inhibition occurs at low percent saturation (n=6).

FIGS. 12A, 12B, and 12C show the effect of natalizumab on adult SCD blood adhesion to VCAM-1. Natalizumab inhibition of total whole blood cells was maximal and similar at 10, 1, and 0.1 μg/mL. After fixing and staining with cell-specific markers, natalizumab was shown to block whole blood leukocytes and reticulocytes with a concentration of drug required for 50% inhibition (IC50) of 0.05±0.03 μg/mL and 0.02±0.02 μg/mL, respectively. Mean±SD, constant flow, 1 dynes/cm².

FIGS. 13A and 13B is a series of comparisons of monovalent and divalent forms of natalizumab in saturation and adhesion assays. In FIG. 13A, the mean EC50 for divalent natalizumab on isolated RBCs from 5 SCD donors was 0.14±0.09 μg/mL whereas the EC50 for monovalent natalizumab was 0.89±0.73 μg/mL or 7-fold higher compared to the divalent form, ranging from 1.7- to 12-fold for individual donors. FIG. 13B is an adhesion inhibition assay, where binding of isolated RBC to VCAM-1 was measured. The IC50 for inhibition of adhesion of reticulocytes in isolated RBC was 0.02±0.02 μg/mL. The IC50 for the monovalent antibody was 0.37±0.33 μg/mL or 19-fold higher compared to the divalent form. Similar to the divalent form, monovalent natalizumab blocked cell adhesion at lower than saturating concentrations. n=5 adult SCD donors, *p<0.05, student T-test, 2-tailed.

FIGS. 14A, 14B, and 14C are a series of predicted natalizumab concentration profiles and α4 integrin saturation profiles for subjects with SCD given prophylactic monthly doses of 150 (FIG. 14A), 300 (FIG. 14B), or 450 mg (FIG. 14C) natalizumab.

FIG. 15 shows predicted hemoglobin profiles (mean and range) after monthly administration of 150, 300, or 450 mg natalizumab with an initial hemoglobin range of 70 to 90 g/L (7 to 9 g/dL).

FIG. 16 shows predicted hemoglobin profiles (mean and range) after monthly administration of 150, 300, or 450 mg natalizumab with an initial hemoglobin range of 80 to 100 g/L (8 to 10 g/dL).

FIG. 17 shows a study design for an exemplary phase 1 multiple-ascending dose study of the safety, tolerability, and pharmacokinetics of intravenous natalizumab in subjects with sickle cell disease.

FIG. 18 shows the effect of IL-1β and TNF-α on the surface levels of endothelial VCAM-1 and ICAM-1 in human umbilical vein endothelial cells (HUVECs) in an adhesion molecule activation assay (6 hours).

FIG. 19 shows the effect of IL-1β and TNF-α on the surface levels of endothelial VCAM-1 and ICAM-1 in HUVECs in an adhesion molecule activation assay (18 hours).

FIG. 20 shows an exemplary workflow for a static adhesion assay for treatment of Jurkat T cells with natalizumab and testing blocked adhesion to TNF-α or IL-1β activated HUVECs.

FIG. 21 shows the effect of natalizumab in blocking Jurkat cell adhesion to HUVECs activated with TNF-α. Jurkat cell adhesion to activated HUVECs was blocked by natalizumab in a dose dependent manner.

FIG. 22 shows a series of microscopy images of natalizumab treated isolated reticulocytes from sickle cell disease (SCD). HUVECs were prepared according to the static adhesion assay workflow.

FIG. 23 shows an exemplary workflow for a fluxion based adhesion assay for treatment of Jurkat T cells with natalizumab and testing blocked adhesion to TNF-α activated HUVECs.

FIG. 24 shows the effect of natalizumab treatment on Jurkat cells on adhesion to HUVEC activated with TNF-α in a fluxion based assay.

FIG. 25 shows the effect of natalizumab treatment on Jurkat cells on adhesion to HUVEC activated with TNF-α in a fluxion based assay.

FIG. 26 shows the effect of natalizumab treatment on SCD whole blood adhesion to HUVECs activated with TNF-α.

### DETAILED DESCRIPTION OF THE INVENTION

The disclosure is based, at least in part, on the discovery that VLA-4 antagonists such as natalizumab are effective in treating subjects suffering from or susceptible to sickle cell disease (SCD).

The following definitions are provided for specific terms used in the following written description and appended claims.

The integrin very late antigen (VLA) superfamily is made up of structurally and functionally related glycoproteins consisting of (alpha and beta) heterodimeric, transmembrane receptor molecules found in various combinations on nearly every mammalian cell type. (For reviews see: E. C. Butcher, Cell, 67, 1033 (1991); D. Cox et al., “The Pharmacology of the Integrins.” Medicinal Research Rev. (1994) and V. W. Engleman et al., “Cell Adhesion Integrins as Pharmaceutical Targets” in Ann. Report in Medicinal Chemistry, Vol. 31, J. A. Bristol, Ed.; Acad. Press, NY, 1996, p. 191). Integrins of the VLA family include (at present) VLA-1, -2, -3, -4, -5, -6, -9, and -11 in which each of the molecules comprise a β1 chain non-covalently bound to an α chain, (α1, α2, α3, α4, α5, α6 and the like), respectively.

Alpha 4 beta 1 (α4β1) integrin is a cell-surface receptor for VCAM-1, fibronectin and possibly other ligands (the latter ligands individually and collectively referred to as “alpha4 ligand(s)”). The term α4β31 integrin (“VLA-4” or “a4b1” or “a4b1 integrin”, used interchangeably herein) refers to polypeptides which are capable of binding to VCAM-1 and members of the extracellular matrix proteins, most particularly fibronectin, or fragments thereof, although it will be appreciated by persons of ordinary skill in the art that other ligands for VLA-4 may exist and can be analyzed using conventional methods. Nevertheless, it is known that the alpha4 subunit will associate with other beta subunits besides betal so the term “alpha 4 integrin” or “alpha 4 subunit-containing integrin”, as used herein, refers to those integrins whose α4 subunit associates with one or another of the beta subunits. Another example of an “α4” integrin besides VLA-4 is alpha4beta7 (α4β7).

Also included in the methods described herein are molecules that antagonize the action of more than one α4 subunit-containing integrin, such as small molecules or antibody molecules that antagonize both VLA-4 and α4β7 or other combinations of α4 subunit-containing integrins. Also included within the scope are methods using a combination of molecules such that the combination antagonizes the action of more than one integrin, such as methods using several small molecules or antibody molecules that in combination antagonize both VLA-4 and α4β7or other combinations of α4 subunit-containing integrins.

“Covalently coupled”, as used herein, refers to moieties (e.g., PEGylated VLA-4 antagonist, immunoglobulin fragment/VLA-4 antagonist) that are either directly covalently bonded to one another, or else are indirectly covalently joined to one another through an intervening moiety or moieties, such as a spacer moiety or moieties. The intervening moiety or moieties are called a “coupling group”. The term “conjugated” is used interchangeably with “covalently coupled”. In this regard a “spacer” refers to a moiety that may be inserted between an amino acid or other component of a VLA-4 antagonist and the remainder of the molecule. A spacer may provide separation between the amino acid or other component and the rest of the molecule so as to prevent the modification from interfering with protein function and/or make it easier for the amino acid or other component to link with another moiety.

“Expression vector,” as used herein refers to a polynucleotide, such as a DNA plasmid or phage (among other common examples) which allows expression of at least one gene when the expression vector is introduced into a host cell. The vector may, or may not, be able to replicate in a cell.

“Functional equivalent” of an amino acid residue is (i) an amino acid having similar reactive properties as the amino acid residue that was replaced by the functional equivalent; (ii) an amino acid of an antagonist of the invention, the amino acid having similar properties as the amino acid residue that was replaced by the functional equivalent; (iii) a non-amino acid molecule having similar properties as the amino acid residue that was replaced by the functional equivalent.

A first polynucleotide encoding a proteinaceous antagonist of the invention is “functionally equivalent” compared with a second polynucleotide encoding the antagonist protein if it satisfies at least one of the following conditions:

(a): the “functional equivalent” is a first polynucleotide that hybridizes to the second polynucleotide under standard hybridization conditions and/or is degenerate to the first polynucleotide sequence. Most preferably, it encodes a mutant protein having the activity of a VLA-4 antagonist protein;

(b) the “functional equivalent” is a first polynucleotide that codes on expression for an amino acid sequence encoded by the second polynucleotide.

The VLA-4 antagonists include, but are not limited to, the agents listed herein as well as their functional equivalents. As used herein, the term “functional equivalent” therefore refers to a VLA-4 antagonist or a polynucleotide encoding the VLA-4 antagonist that has the same or an improved beneficial effect on the recipient as the VLA-4 antagonist of which it is deemed a functional equivalent. As will be appreciated by one of ordinary skill in the art, a functionally equivalent protein can be produced by recombinant techniques, e.g., by expressing a “functionally equivalent DNA”. Accordingly, the disclosure embraces integrin proteins encoded by naturally-occurring DNAs, as well as by non-naturally-occurring DNAs which encode the same protein as encoded by the naturally-occurring DNA. Due to the degeneracy of the nucleotide coding sequences, other polynucleotides may be used to encode integrin protein. These include all, or portions of the above sequences which are altered by the substitution of different codons that encode the same amino acid residue within the sequence, thus producing a silent change. Such altered sequences are regarded as equivalents of these sequences. For example, Phe (F) is coded for by two codons, TTC or TTT, Tyr (Y) is coded for by TAC or TAT and His (H) is coded for by CAC or CAT. On the other hand, Trp (W) is coded for by a single codon, TGG. Accordingly, it will be appreciated that for a given DNA sequence encoding a particular integrin there will be many DNA degenerate sequences that will code for it. These degenerate DNA sequences are considered within the scope of this disclosure.

The term “chimeric”, when referring to an antagonist, means that the antagonist is comprised of a linkage (chemical cross-linkage or covalent or other type) of two or more proteins having disparate structures and/or having disparate sources of origin. Thus, a chimeric VLA-4 antagonist may include one moiety that is a VLA-4 antagonist or fragment and another moiety that is not a VLA-4 antagonist.

A species of “chimeric” protein is a “fusion” or “fusion protein” which refers to a co-linear, covalent linkage of two or more proteins or fragments thereof via their individual peptide backbones, most preferably through genetic expression of a polynucleotide molecule encoding those proteins. Thus, preferred fusion proteins are chimeric proteins that include a VLA-4 antagonist or fragment covalently linked to a second moiety that is not a VLA-4 antagonist. Preferred fusion proteins include portions of intact antibodies that retain antigen-binding specificity, for example, Fab fragments, Fab′ fragments, F(ab′)2 fragments, F(v) fragments, heavy chain monomers or dimers, light chain monomers or dimers, dimers consisting of one heavy and one light chain, and the like.

The other preferred fusion proteins are chimeric and comprise a VLA-4 antagonist moiety fused or otherwise linked to all or part of the hinge and constant regions of an immunoglobulin light chain, heavy chain, or both. Thus, the methods described herein can utilize a molecule that include: (1) an VLA-4 antagonist moiety, (2) a second peptide, e.g., one which increases solubility or in vivo life time of the VLA-4 antagonist moiety, e.g., a member of the immunoglobulin super family or fragment or portion thereof, e.g., a portion or a fragment of IgG, e.g., the human IgG1 heavy chain constant region, e.g., CH2, CH3, and hinge regions. Specifically, a “VLA-4 antagonist/lg fusion” is a protein comprising a biologically active VLA-4 antagonist (e.g. a soluble VLA-4 ligand), or a biologically active fragment thereof linked to an N-terminus of an immunoglobulin chain wherein a portion of the N-terminus of the immunoglobulin is replaced with the VLA-4 antagonist. A species of VLA-4 antagonist/lg fusion is a “VLA-4/Fc fusion” which is a protein comprising a VLA-4 antagonist, e.g., described herein, linked to at least a part of the constant domain of an immunoglobulin. A preferred Fc fusion comprises a VLA-4 antagonist, e.g., described herein, linked to a fragment of an antibody containing the C terminal domain of the heavy immunoglobulin chains.

The term “fusion protein” also means a VLA-4 antagonist chemically linked via a mono- or hetero-functional molecule to a second moiety that is not a VLA-4 antagonist (resulting in a “chimeric” molecule). Thus, one example of a chemically linked, as opposed to recombinantly linked, chimeric molecule that is a fusion protein may comprise: (1) VLA-4 subunit targeting moiety, e.g., a VCAM-1 moiety capable of) binding to VLA-4) on the surface of VLA-4 bearing cells; (2) a second molecule which increases solubility or in vivo life time of the targeting moiety, e.g., a polyalkylene glycol polymer such as polyethylene glycol (PEG). The VLA-4 targeting moiety can be any naturally occurring VLA-4 ligand or fragment thereof, e.g., a VCAM-1 peptide or a similar conservatively substituted amino acid sequence.

Calculations of “homology” or “sequence identity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frameshift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences.

As used herein, the term “hybridizes under high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. High stringency hybridization conditions include hybridization in 6× SSC at about 45° C., followed by one or more washes in 0.2 x SSC, 0.1% SDS at 65° C., or substantially similar conditions.

“Isolated” (used interchangeably with “substantially pure”), when applied to nucleic acid i.e., polynucleotide sequences that encode VLA antagonists, means an RNA or DNA polynucleotide, portion of genomic polynucleotide, cDNA or synthetic polynucleotide which, by virtue of its origin or manipulation: (i) is not associated with all of a polynucleotide with which it is associated in nature (e.g., is present in a host cell as an expression vector, or a portion thereof); or (ii) is linked to a nucleic acid or other chemical moiety other than that to which it is linked in nature; or (iii) does not occur in nature. By “isolated” it is further meant a polynucleotide sequence that is: (i) amplified in vitro by, for example, polymerase chain reaction (PCR); (ii) synthesized chemically; (iii) produced recombinantly by cloning; or (iv) purified, as by cleavage and gel separation. Thus, “substantially pure nucleic acid” is a nucleic acid which is not immediately contiguous with one or both of the coding sequences with which it is normally contiguous in the naturally occurring genome of the organism from which the nucleic acid is derived. Substantially pure DNA also includes a recombinant DNA which is part of a hybrid gene encoding additional integrin sequences.

“Isolated” (used interchangeably with “substantially pure”), when applied to polypeptides means a polypeptide or a portion thereof which, by virtue of its origin or manipulation: (i) is present in a host cell as the expression product of a portion of an expression vector; or (ii) is linked to a protein or other chemical moiety other than that to which it is linked in nature; or (iii) does not occur in nature, for example, a protein that is chemically manipulated by appending, or adding at least one hydrophobic moiety to the protein so that the protein is in a form not found in nature. By “isolated” it is further meant a protein that is: (i) synthesized chemically; or (ii) expressed in a host cell and purified away from associated and contaminating proteins. The term generally means a polypeptide that has been separated from other proteins and nucleic acids with which it naturally occurs. Preferably, the polypeptide is also separated from substances such as antibodies or gel matrices (polyacrylamide) which are used to purify it.

A “pharmacological agent” is defined as one or more compounds or molecules or other chemical entities administered to a subject (in addition to the VLA-4 antagonists) that affects the action of the antagonist. The term “pharmacological agent” as used herein refers to such an agent(s) that are administered during “combination therapy” where the VLA-4 antagonist is administered either prior to, after, or simultaneously with, administration of one or more pharmacological agents.

“Protein,” as used herein refers to any polymer consisting essentially of any of the 20 amino acids. Although “polypeptide” is often used in reference to relatively large polypeptides, and “peptide” is often used in reference to small polypeptides, usage of these terms in the art overlaps and is varied. The term “protein” as used herein refers to peptides, proteins and polypeptides, unless otherwise noted.

The terms “peptide(s)”, “protein(s)” and “polypeptide(s)” are used interchangeably herein. The terms “polynucleotide sequence” and “nucleotide sequence” are also used interchangeably herein.

“Recombinant,” as used herein, means that a protein is derived from recombinant, mammalian expression systems. Since integrin is not glycosylated nor contains disulfide bonds, it can be expressed in most prokaryotic and eukaryotic expression systems.

“Small molecule” VLA-4 antagonist refers to chemical agents (i.e., organic molecules) capable of disrupting the integrin/integrin ligand interaction by, for instance, blocking VLA-4/VCAM interactions by binding VLA-4 on the surface of cells or binding VCAM-1 on the surface of cells. Such small molecules may also bind respective VLA-4 and VCAM-1 receptors. VLA-4 and VCAM-1 small molecule inhibitors may themselves be peptides, semi-peptidic compounds or non-peptidic compounds, such as small organic molecules that are antagonists of the VCAM-1/VLA-4 interaction.

A VLA-4 antagonist (and a therapeutic composition comprising the same) is said to have “therapeutic efficacy,” and an amount of the agent is said to be “therapeutically effective,” if administration of that amount of the agent is sufficient to cause a clinically significant improvement in SCD (e.g., a decrease in vaso-occlusive (VOC) events, a decreased duration of VOC events, an increase in hemoglobin levels, an improvement of patient-reported fatigue, a decrease in pain, a decrease in lactate dehydrogenase, an increase in reticulocytes, and/or a decrease in anemia).

The term “treating”, as used herein, refers to administering a therapy in an amount, manner (e.g., schedule of administration), and/or mode (e.g., route of administration), effective to improve a disorder or a symptom thereof, or to prevent or slow the progression of a disorder or a symptom thereof. This can be evidenced by, e.g., an improvement in a parameter associated with a disorder or a symptom thereof, e.g., to a statistically significant degree or to a degree detectable to one skilled in the art. An effective amount, manner, or mode can vary depending on the subject and may be tailored to the subject. By preventing or slowing progression of a disorder or a symptom thereof, a treatment can prevent or slow deterioration resulting from a disorder or a symptom thereof in an affected or diagnosed subject.

The term “biologic” refers to a protein-based therapeutic agent. In a preferred embodiment, the biologic is at least 30, 40, 50 or 100 amino acid residues in length.

A “VLA-4 binding agent” refers to any compound that binds to VLA-4 integrin with a Ka of less than 10⁻⁶ M. An example of a VLA-4 binding agent is a VLA-4 binding protein, e.g., a VLA-4 binding antibody such as natalizumab.

A “VLA-4 antagonist” refers to any compound that at least partially inhibits an activity of a VLA-4 integrin, particularly a binding activity of a VLA-4 integrin or a signaling activity, e.g., ability to transduce a VLA-4 mediated signal. For example, a VLA-4 antagonist may inhibit binding of VLA-4 to a cognate ligand of VLA-4, e.g., a cell surface protein such as VCAM-1, or to an extracellular matrix component, such as fibronectin or osteopontin. A typical VLA-4 antagonist can bind to VLA-4 or to a VLA-4 ligand, e.g., VCAM-1 or an extracellular matrix component, such as fibronectin or osteopontin. A VLA-4 antagonist that binds to VLA-4 may bind to either the α4 subunit or the β1 subunit, or to both. A VLA-4 antagonist may also interact with other α4 subunit containing integrins (e.g., α4β7) or with other β1 containing integrins. A VLA-4 antagonist may bind to VLA-4 or to a VLA-4 ligand with a K_{d} of less than 10⁻⁶, 10⁻⁷, 10⁻⁸, 10⁻⁹, or 10⁻¹⁰ M.

The term “antibody molecule” refers to an antibody or antigen binding fragment thereof. As used herein, the term “antibody” refers to a protein that includes at least one immunoglobulin variable region, e.g., an amino acid sequence that provides an immunoglobulin variable domain or immunoglobulin variable domain sequence. For example, an antibody can include a heavy (H) chain variable region (abbreviated herein as VH), and/or a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab fragments, F(ab′)2 fragments, Fd fragments, Fv fragments, and dAb fragments) as well as complete antibodies, e.g., intact immunoglobulins of types IgA, IgG, IgE, IgD, IgM (as well as subtypes thereof). The light chains of the immunoglobulin may be of types kappa or lambda. In one embodiment, the antibody is glycosylated. An antibody can be functional for antibody-dependent cytotoxicity and/or complement-mediated cytotoxicity, or may be non-functional for one or both of these activities.

The VH and VL regions can be further subdivided into regions of hypervariability, termed “complementarity determining regions” (“CDR”), interspersed with regions that are more conserved, termed “framework regions” (FR). The extent of the FR's and CDR's has been precisely defined (see, Kabat, E. A., et al. (1991) Sequences of Proteins of Immunological Interest, Fifth Edition, US Department of Health and Human Services, NIH Publication No. 91-3242; and Chothia, C. et al. (1987) J. Mol. Biol. 196:901-917). Kabat definitions are used herein. Each VH and VL is typically composed of three CDR's and four FR's, arranged from amino-terminus to carboxyl-terminus in the following order: FR1, CDR1, FR2, CDR2, FR3, CDR3, FR4.

An “immunoglobulin domain” refers to a domain from the variable or constant domain of immunoglobulin molecules Immunoglobulin domains typically contain two β-sheets formed of about seven β-strands, and a conserved disulphide bond (see, e.g., A. F. Williams and A. N. Barclay 1988 Ann. Rev Immunol. 6:381-405).

As used herein, an “immunoglobulin variable domain sequence” refers to an amino acid sequence that can form the structure of an immunoglobulin variable domain. For example, the sequence may include all or part of the amino acid sequence of a naturally-occurring variable domain. For example, the sequence may omit one, two or more N- or C-terminal amino acids, internal amino acids, may include one or more insertions or additional terminal amino acids, or may include other alterations. In one embodiment, a polypeptide that includes an immunoglobulin variable domain sequence can associate with another immunoglobulin variable domain sequence to form a target binding structure (or “antigen binding site”), e.g., a structure that interacts with VLA-4.

The VH or VL chain of the antibody can further include all or part of a heavy or light chain constant region, to thereby form a heavy or light immunoglobulin chain, respectively. In one embodiment, the antibody is a tetramer of two heavy immunoglobulin chains and two light immunoglobulin chains. The heavy and light immunoglobulin chains can be connected by disulfide bonds. The heavy chain constant region typically includes three constant domains, CH₁, CH₂ and CH₃. The light chain constant region typically includes a CL domain. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system.

One or more regions of an antibody can be human, effectively human, or humanized. For example, one or more of the variable regions can be human or effectively human. For example, one or more of the CDRs, e.g., HC CDR1, HC CDR2, HC CDR3, LC CDR1, LC CDR2, and LC CDR3, can be human. Each of the light chain CDRs can be human. HC CDR3 can be human. One or more of the framework regions can be human, e.g., FR1, FR2, FR3, and FR4 of the HC or LC. In one embodiment, all the framework regions are human, e.g., derived from a human somatic cell, e.g., a hematopoietic cell that produces immunoglobulins or a non-hematopoietic cell. In one embodiment, the human sequences are germline sequences, e.g., encoded by a germline nucleic acid. One or more of the constant regions can be human, effectively human, or humanized. In another embodiment, at least 70, 75, 80, 85, 90, 92, 95, or 98% of the framework regions (e.g., FR1, FR2, and FR3, collectively, or FR1, FR2, FR3, and FR4, collectively) or the entire antibody can be human, effectively human, or humanized. For example, FR1, FR2, and FR3 collectively can be at least 70, 75, 80, 85, 90, 92, 95, 98, or 99% identical to a human sequence encoded by a human germline segment.

An “effectively human” immunoglobulin variable region is an immunoglobulin variable region that includes a sufficient number of human framework amino acid positions such that the immunoglobulin variable region does not elicit an immunogenic response in a normal human. An “effectively human” antibody is an antibody that includes a sufficient number of human amino acid positions such that the antibody does not elicit an immunogenic response in a normal human.

A “humanized” immunoglobulin variable region is an immunoglobulin variable region that is modified such that the modified form elicits less of an immune response in a human than does the non-modified form, e.g., is modified to include a sufficient number of human framework amino acid positions such that the immunoglobulin variable region does not elicit an immunogenic response in a normal human. Descriptions of “humanized” immunoglobulins include, for example, U.S. Pat. No.: 6,407,213 and U.S. Pat. No.: 5,693,762. In some cases, humanized immunoglobulins can include a non-human amino acid at one or more framework amino acid positions.

All or part of an antibody can be encoded by an immunoglobulin gene or a segment thereof Exemplary human immunoglobulin genes include the kappa, lambda, alpha (IgA1 and IgA2), gamma (IgG1, IgG2, IgG3, IgG4), delta, epsilon and mu constant region genes, as well as the myriad immunoglobulin variable region genes. Full-length immunoglobulin “light chains” (about 25 Kd or 214 amino acids) are encoded by a variable region gene at the NH2-terminus (about 110 amino acids) and a kappa or lambda constant region gene at the COOH-terminus. Full-length immunoglobulin “heavy chains” (about 50 Kd or 446 amino acids), are similarly encoded by a variable region gene (about 116 amino acids) and one of the other aforementioned constant region genes, e.g., gamma (encoding about 330 amino acids).

The term “antigen-binding fragment” of a full length antibody refers to one or more fragments of a full-length antibody that retain the ability to specifically bind to a target of interest, e.g., VLA-4. Examples of binding fragments encompassed within the term “antigen-binding fragment” of a full length antibody include (i) a Fab fragment, a monovalent fragment consisting of the VL, VH, CL and CH1 domains; (ii) a F(ab′).sub.2 fragment, a bivalent fragment including two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:54-546), which consists of a VH domain; and (vi) an isolated complementarity determining region (CDR) that retains functionality. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules known as single chain Fv (scFv). See, e.g., Bird et al. (1988) Science 242:423-426; and Huston et al. (1988) Proc. Natl. Acad. Sci. USA 85:5879-5883.

Practice of the present invention will employ, unless indicated otherwise, conventional techniques of cell biology, cell culture, molecular biology, microbiology, recombinant DNA, protein chemistry, pharmacology and immunology, which are within the skill of the art. Such techniques are described in the literature. Unless stipulated otherwise, all references cited in the Detailed Description are incorporated herein by reference.

### VLA-4 Antagonists

A VLA-4 antagonist is an antagonist of interactions of α4 integrins with their ligands, such as the VCAM-1/VLA-4 interaction. This is an agent, e.g., a polypeptide or other molecule, which can inhibit or block VCAM-1 and/or VLA-4-mediated binding or which can otherwise modulate VCAM-1 and/or VLA-4 function, e.g., by inhibiting or blocking VLA-4-ligand mediated VLA-4 signal transduction or VCAM-1-ligand mediated VCAM-1 signal transduction and which is effective in the treatment of SCD, preferably in the same manner as anti-VLA-4 binding agents such as anti-VLA-4 antibodies.

A VLA-4 antagonist can have one or more of the following properties: (1) it coats, or binds to, VLA-4 on the surface of a VLA-4 bearing cell (e.g., an endothelial cell) with sufficient specificity to inhibit a VLA-4-ligand/VLA-4 interaction, e.g., the VCAM-1/VLA-4 interaction; (2) it coats, or binds to, VLA-4 on the surface of a VLA-4 bearing cell (i.e., a lymphocyte) with sufficient specificity to modify, and preferably to inhibit, transduction of a VLA-4-mediated signal e.g., VLA-4/VCAM-1-mediated signaling; (3) it coats, or binds to, a VLA-4-ligand, (e.g., VCAM-1) on endothelial cells with sufficient specificity to inhibit the VLA-4/VCAM-1 interaction; (4) it coats, or binds to, a VLA-4-ligand (e.g., VCAM-1) with sufficient specificity to modify, and preferably to inhibit, transduction of VLA-4-ligand mediated VLA-4 signaling, e.g., VCAM-1-mediated VLA-4 signaling. In preferred embodiments the antagonist has one or both of properties 1 and 2. In other preferred embodiments the antagonist has one or both of properties 3 and 4. Moreover, more than one antagonist can be administered to a patient, e.g., an agent which binds to VLA-4 can be combined with an agent which binds to VCAM-1.

For example, antibody molecules as well as soluble forms of the natural binding proteins for VLA-4 and VCAM-1 are useful.

### VLA-4 Antagonist Antibody Molecules

Natalizumab, an α4 integrin binding antibody, inhibits the migration of leukocytes from the blood. Natalizumab binds to VLA-4 on the surface of activated T-cells and other mononuclear leukocytes. It can disrupt adhesion between the T-cell and endothelial cells, and thus prevent migration of mononuclear leukocytes across the endothelium and into the parenchyma. As a result, the levels of proinflammatory cytokines can also be reduced.

Natalizumab and related VLA-4 binding antibodies are described, e.g., in U.S. Pat. No.: 5,840,299. Monoclonal antibodies 21.6 and HP1/2 are exemplary murine monoclonal antibodies that bind VLA-4. Natalizumab is a humanized version of murine monoclonal antibody 21.6 (see, e.g., U.S. Pat. No.: 5,840,299). A humanized version of HP1/2 has also been described (see, e.g., U.S. Pat. No.: 6,602,503). Several additional VLA-4 binding monoclonal antibodies, such as HP2/1, HP2/4, L25 and P4C2, are described, e.g., in U.S. Pat. No.: 6,602,503; Sanchez-Madrid et al., 1986 Eur. J. mmunol., 16:1343-1349; Hemler et al., 1987 J. Biol. Chem. 2:11478-11485; Issekutz and Wykretowicz, 1991, J. Immunol., 147: 109 (TA-2 mab); Pulido et al., 1991 J. Biol. Chem., 266(16):10241-10245; and U.S. Pat. No. 5,888,507.

Some VLA-4 binding antibody molecules recognize epitopes of the α4 subunit that are involved in binding to a cognate ligand, e.g., VCAM-1 or fibronectin. Many such antibody molecules inhibit binding of VLA-4 to cognate ligands (e.g., VCAM-1 and fibronectin).

Some useful VLA-4 binding antibodies can interact with VLA-4 on cells, e.g., lymphocytes, but do not cause cell aggregation. However, other VLA-4 binding antibodies have been observed to cause such aggregation. HP1/2 does not cause cell aggregation. The HP1/2 monoclonal antibody (Sanchez-Madrid et al., 1986) has an extremely high potency, blocks VLA-4 interaction with both VCAM1 and fibronectin, and has the specificity for epitope B on VLA-4. This antibody and other B epitope-specific antibodies (such as B1 or B2 epitope binding antibodies; Pulido et al., 1991, supra) represent one class of VLA-4 binding antibodies that can be used in the methods described herein. Antibodies that compete for binding with a VLA-4 binding antibody, e.g., natalizumab, can also be used in the methods described herein.

An exemplary VLA-4 binding antibody molecule has one or more CDRs, e.g., all three HC CDRs and/or all three LC CDRs of a particular antibody disclosed herein, or CDRs that are, in sum, at least 80, 85, 90, 92, 94, 95, 96, 97, 98, 99% identical to such an antibody, e.g., natalizumab. In one embodiment, the H1 and H2 hypervariable loops have the same canonical structure as those of an antibody described herein. In one embodiment, the L1 and L2 hypervariable loops have the same canonical structure as those of an antibody molecule described herein.

In one embodiment, the amino acid sequence of the HC and/or LC variable domain sequence is at least 70, 80, 85, 90, 92, 95, 97, 98, 99, or 100% identical to the amino acid sequence of the HC and/or LC variable domain of an antibody described herein, e.g., natalizumab. The amino acid sequence of the HC variable domain (see, e.g., SEQ ID NO: 1) and/or LC variable domain (see, e.g., SEQ ID NO: 2) can differ by at least one amino acid, but no more than ten, eight, six, five, four, three, or two amino acids from the corresponding sequence of an antibody described herein, e.g., natalizumab. For example, the differences may be primarily or entirely in the framework regions.

Exemplary amino acid sequences of the light chain variable domain (SEQ ID NO: 2) and the heavy chain variable domain (SEQ ID NO: 1) of natalizumab are shown in Table 1. CDR sequences are underlined.

TABLE 1
Exemplary natalizumab HC variable domain and LC
variable domain sequences

| | Amino Acid Sequence |
|---|---|
| Natalizumab | QVQLVQSGAEVKKPGASVKVSCKASGFNIKDTYIHW |
| Heavy | VRQAPGQRLEWMGRIDPANGYTKYDPKFQGRVTITA |
| Chain | DTSASTAYMELSSLRSEDTAVYYCAREGYYGNYGVY |
| (SEQ ID | AMDYWGQGTLVTVSS |
| NO: 1) | |
| Natalizumab | DIQMTQSPSSLSASVGDRVTITCKTSQDINKYMAWY |
| Light Chain | QQTPGKAPRLLIHYTSALQPGIPS |
| (SEQ ID | RFSGSGSGRDYTFTISSLQPEDIATYYCLQ |
| NO: 2) | YDNLWTFGQGTKVEIKRTV |

The amino acid sequences of the HC and LC variable domain sequences can be encoded by a nucleic acid sequence that hybridizes under high stringency conditions to a nucleic acid sequence described herein or one that encodes a variable domain or an amino acid sequence described herein. In one embodiment, the amino acid sequences of one or more framework regions (e.g., FR1, FR2, FR3, and/or FR4) of the HC and/or LC variable domain are at least 70, 80, 85, 90, 92, 95, 97, 98, 99, or 100% identical to corresponding framework regions of the HC and LC variable domains of an antibody described herein. In one embodiment, one or more heavy or light chain framework regions (e.g., HC FR1, FR2, and FR3) are at least 70, 80, 85, 90, 95, 96, 97, 98, or 100% identical to the sequence of corresponding framework regions from a human germline antibody.

### Other VLA-4 Antagonist Polypeptides

In some embodiments, the VLA-4 antagonist can be a soluble form of a ligand. Soluble forms of the ligand proteins include soluble VCAM-1 or fibronectin peptides, VCAM-1 fusion proteins, or bifunctional VCAM-1/Ig fusion proteins. For example, a soluble form of a VLA-4 ligand or a fragment thereof may be administered to bind to VLA-4, and in some instances, compete for a VLA-4 binding site on cells, thereby leading to effects similar to the administration of antagonists such as anti-VLA-4 antibodies. For example, soluble VLA-4 integrin mutants that bind VLA-4 ligand but do not elicit integrin-dependent signaling are suitable for use in the described methods. Such mutants can act as competitive inhibitors of wild type integrin protein and are considered “antagonists.” Soluble forms of the natural binding proteins for VLA-4 include soluble VCAM-1 peptides, VCAM-1 fusion proteins, bifunctional VCAM-1/lg fusion proteins (e.g. “chimeric” molecules, discussed above), fibronectin, fibronectin having an alternatively spliced non-type III connecting segment, and fibronectin peptides containing the amino acid sequence EILDV or a similar conservatively substituted amino acid sequence. As used herein, a “soluble VLA-4 peptide” or a “soluble VCAM-1 peptide” is a VLA-4 or VCAM-1 polypeptide incapable of anchoring itself in a membrane. Such soluble polypeptides include, for example, VLA-4 and VCAM polypeptides that lack a sufficient portion of their membrane spanning domain to anchor the polypeptide or are modified such that the membrane spanning domain is non-functional. These binding agents can act by competing with the cell-surface binding protein for VLA-4 or by otherwise altering VLA-4 function. For example, a soluble form of VCAM-1 (see, e.g., Osborn et al. 1989, Cell, 59: 1203-1211) or a fragment thereof may be administered to bind to VLA-4, and preferably compete for a VLA-4 binding site on VCAM-1-bearing cells, thereby leading to effects similar to the administration of antagonists such as small molecules or anti-VLA-4 antibodies.

### Small Molecule VLA-4 Antagonists

“Small molecules” are agents that mimic the action of peptides to disrupt VLA-4/ligand interactions by, for instance, binding VLA-4 and blocking interaction with a VLA-4 ligand (e.g., VCAM-1 or fibronectin), or by binding a VLA-4 ligand and preventing the ligand from interacting with VLA-4. One exemplary small molecule is an oligosaccharide that mimics the binding domain of a VLA-4 ligand (e.g., fibronectin or VCAM-1) and binds the ligand-binding domain of VLA-4. (See, Devlin et al., Science 249: 400-406 (1990); Scott and Smith, Science 249:386-390 (1990); and U.S. Pat. No. 4,833,092 (Geysers), all incorporated herein by reference.)

A “small molecule” may be chemical compound, e.g., an organic compound, or a small peptide, or a larger peptide-containing organic compound or non-peptidic organic compound. A “small molecule” is not intended to encompass an antibody or antibody fragment. Although the molecular weight of small molecules is generally less than 2000 Daltons, this figure is not intended as an absolute upper limit on molecular weight.

Examples of other small molecules useful in the invention can be found in Komoriya et al. (“The Minimal Essential Sequence for a Major Cell Type-Specific Adhesion Site (CS1) Within the Alternatively Spliced Type III Connecting Segment Domain of Fibronectin Is Leucine-Aspartic Acid-Valine”, J. Biol. Chem., 266 (23), pp. 15075-79 (1991)). They identified the minimum active amino acid sequence necessary to bind VLA-4 and synthesized a variety of overlapping peptides based on the amino acid sequence of the CS-1 region (the VLA-4 binding domain) of a particular species of fibronectin. They identified an 8-amino acid peptide, Glu-Ile-Leu-Asp-Val-Pro-Ser-Thr, as well as two smaller overlapping pentapeptides, Glu-Ile-Leu-Asp-Val and Leu-Asp-Val-Pro-Ser, which possessed inhibitory activity against fibronectin-dependent cell adhesion. Certain larger peptides containing the LDV sequence were subsequently shown to be active in vivo (T. A. Ferguson et al., “Two integrin Binding Peptides Abrogate T-cell-Mediated Immune Responses In Vivo”, Proc. Natl. Acad. Sci. USA, 88, pp. 8072-76 (1991); and S. M. Wahl et al., “Synthetic Fibronectin Peptides Suppress Arthritis in Rats by Interrupting Leukocyte Adhesion and Recruitment”, J. Clin. Invest., 94, pp. 655-62 (1994)). A cyclic pentapeptide, Arg-Cys-Asp-TPro-Cys (wherein TPro denotes 4-thioproline), which can inhibit both VLA-4 and VLA-5 adhesion to fibronectin has also been described. (See, e.g., D. M. Nowlin et al. “A Novel Cyclic Pentapeptide Inhibits Alpha4Beta1 Integrin-mediated Cell Adhesion”, J. Biol. Chem., 268(27), pp. 20352-59 (1993); and PCT publication PCT/US91/04862). This pentapeptide was based on the tripeptide sequence Arg-Gly-Asp from fibronectin which had been known as a common motif in the recognition site for several extracellular-matrix proteins. Examples of other VLA-4 inhibitors have been reported, for example, in Adams et al. “Cell Adhesion Inhibitors”, PCT US97/13013, describing linear peptidyl compounds containing beta-amino acids which have cell adhesion inhibitory activity. International patent applications WO 94/15958 and WO 92/00995 describe cyclic peptide and peptidomimetic compounds with cell adhesion inhibitory activity. International patent applications WO 93/08823 and WO 92/08464 describe guanidinyl-, urea- and thiourea-containing cell adhesion inhibitory compounds. U.S. Pat. No. 5,260,277 describes guanidinyl cell adhesion modulation compounds. Other peptidyl antagonists of VLA-4 have been described in D. Y. Jackson et al., “Potent α4β1peptide antagonists as potential anti-inflammatory agents”, J. Med. Chem., 40,3359 (1997); H. Shroff et al., “Small peptide inhibitors of α4β7 mediated MadCAM-1 adhesion to lymphocytes”, Bio. Med, Chem. Lett., 1 2495 (1996); U.S. Pat. No. 5,510,332, PCT Publications WO 98/53814, WO97/03094, WO97/02289, WO96/40781, WO96/22966, WO96/20216, WO96/01644, WO96106108, and W095/15973, and others.

Such small molecule agents may be produced by synthesizing a plurality of peptides (e.g., 5 to 20 amino acids in length), semi-peptidic compounds or non-peptidic, organic compounds, and then screening those compounds for their ability to inhibit the VLA-4/VCAM interaction. See generally U.S. Pat. No. 4,833,092, Scott and Smith, “Searching for Peptide Ligands with an Epitope Library”, Science, 249, pp. 386-90 (1990), and Devlin et al., “Random Peptide Libraries: A Source of Specific Protein Binding Molecules”, Science, 249, pp. 40407 (1990).

### Antibody Generation

Antibodies that bind to VLA-4 can be generated by immunization, e.g., using an animal, or by in vitro methods such as phage display. All or part of VLA-4 can be used as an immunogen. For example, the extracellular region of the α4 subunit can be used as an immunogen. In one embodiment, the immunized animal contains immunoglobulin producing cells with natural, human, or partially human immunoglobulin loci. In one embodiment, the non-human animal includes at least a part of a human immunoglobulin gene. For example, it is possible to engineer mouse strains deficient in mouse antibody production with large fragments of the human Ig loci. Using the hybridoma technology, antigen-specific monoclonal antibodies derived from the genes with the desired specificity may be produced and selected. See, e.g., XenoMouse™, Green et al., Nature Genetics 7:13-21 (1994), US 2003-0070185, U.S. Pat. No.: 5,789,650, and WO 96/34096.

Non-human antibodies to VLA-4 can also be produced, e.g., in a rodent. The non-human antibody can be humanized, e.g., as described in U.S. Pat. No.: 6,602,503, EP 239 400, U.S. Pat. No.: 5,693,761, and U.S. Pat. No.: 6,407,213.

EP 239 400 (Winter et al.) describes altering antibodies by substitution (within a given variable region) of their complementarity determining regions (CDRs) for one species with those from another. CDR-substituted antibodies can be less likely to elicit an immune response in humans compared to true chimeric antibodies because the CDR-substituted antibodies contain considerably less non-human components (Riechmann et al., 1988, Nature 332, 323-327; Verhoeyen et al., 1988, Science 239, 1534-1536). Typically, CDRs of a murine antibody substituted into the corresponding regions in a human antibody by using recombinant nucleic acid technology to produce sequences encoding the desired substituted antibody. Human constant region gene segments of the desired isotype (usually gamma I for CH and kappa for CL) can be added and the humanized heavy and light chain genes can be co-expressed in mammalian cells to produce soluble humanized antibody.

Queen et al. (Proc. Natl. Acad. Sci. U.S.A. 86:10029-33, 1989) and WO 90/07861 have described a process that includes choosing human V framework regions by computer analysis for optimal protein sequence homology to the V region framework of the original murine antibody, and modeling the tertiary structure of the murine V region to visualize framework amino acid residues that are likely to interact with the murine CDRs. These murine amino acid residues are then superimposed on the homologous human framework. See also U.S. Pat. Nos.: 5,693,762; 5,693,761; 5,585,089; and 5,530,101. Tempest et al., 1991, Biotechnology 9:266-271, utilize, as standard, the V region frameworks derived from NEWM and REI heavy and light chains, respectively, for CDR-grafting without radical introduction of mouse residues. An advantage of using the Tempest et al. approach to construct NEWM and REI based humanized antibodies is that the three dimensional structures of NEWM and REI variable regions are known from x-ray crystallography and thus specific interactions between CDRs and V region framework residues can be modeled.

Non-human antibodies can be modified to include substitutions that insert human immunoglobulin sequences, e.g., consensus human amino acid residues at particular positions, e.g., at one or more (preferably at least five, ten, twelve, or all) of the following positions: (in the FR of the variable domain of the light chain) 4L, 35L, 36L, 38L, 43L, 44L, 58L, 46L, 62L, 63L, 64L, 65L, 66L, 67L, 68L, 69L, 70L, 71L, 73L, 85L, 87L, 98L, and/or (in the FR of the variable domain of the heavy chain) 2H, 4H, 24H, 36H, 37H, 39H, 43H, 45H, 49H, 58H, 60H, 67H, 68H, 69H, 70H, 73H, 74H, 75H, 78H, 91H, 92H, 93H, and/or 103H (according to the Kabat numbering). See, e.g., U.S. Pat. No. 6,407,213.

Fully human monoclonal antibodies that bind to VLA-4 can be produced, e.g., using in vitro-primed human splenocytes, as described by Boerner et al., 1991, J. Immunol., 147, 86-95. They may be prepared by repertoire cloning as described by Persson et al., 1991, Proc. Nat. Acad. Sci. USA, 88: 2432-2436 or by Huang and Stollar, 1991, J. Immunol. Methods 141, 227-236; also U.S. Pat. No. 5,798,230. Large nonimmunized human phage display libraries may also be used to isolate high affinity antibodies that can be developed as human therapeutics using standard phage technology (see, e.g., Vaughan et al, 1996; Hoogenboom et al. (1998) Immunotechnology 4:1-20; and Hoogenboom et al. (2000) Immunol Today 2:371-8; US 2003-0232333). Transgenic animals, e.g., transgenic mice, expressing human antibody gene sequences may be used to produce human monoclonal antibodies using technology as described in, e.g., Lonberg N. (2005) Nat. Biotechnol. 23(9):1117-25.

### Antibody Production

Antibodies can be produced in prokaryotic and eukaryotic cells. In one embodiment, the antibodies (e.g., scFv's) are expressed in a yeast cell such as Pichia (see, e.g., Powers et al. (2001) J Immunol Methods. 251:123-35), Hanseula, or Saccharomyces.

In one embodiment, antibodies, particularly full length antibodies, e.g., IgG's, are produced in mammalian cells. Exemplary mammalian host cells for recombinant expression include Chinese Hamster Ovary (CHO cells) (including dhfr− CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), lymphocytic cell lines, e.g., NSO myeloma cells and SP2 cells, COS cells, K562, and a cell from a transgenic animal, e.g., a transgenic mammal For example, the cell is a mammary epithelial cell.

In addition to the nucleic acid sequence encoding the immunoglobulin domain, the recombinant expression vectors may carry additional nucleic acid sequences, such as sequences that regulate replication of the vector in host cells (e.g., origins of replication) and selectable marker genes. The selectable marker gene facilitates selection of host cells into which the vector has been introduced (see, e.g., U.S. Pat. Nos.: 4,399,216, 4,634,665 and 5,179,017). Exemplary selectable marker genes include the dihydrofolate reductase (DHFR) gene (for use in dhfr⁻ host cells with methotrexate selection/amplification) and the neo gene (for G418 selection).

In an exemplary system for recombinant expression of an antibody (e.g., a full length antibody or an antigen-binding portion thereof), a recombinant expression vector encoding both the antibody heavy chain and the antibody light chain is introduced into dhfr⁻ CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, to transfect the host cells, to select for transformants, to culture the host cells, and to recover the antibody from the culture medium. For example, some antibodies can be isolated by affinity chromatography with a Protein A or Protein G.

Antibodies may also include modifications, e.g., modifications that alter Fc function, e.g., to decrease or remove interaction with an Fc receptor or with Clq, or both. For example, the human IgG1 constant region can be mutated at one or more residues, e.g., one or more of residues 234 and 237, e.g., according to the numbering in U.S. Pat. No.: 5,648,260. Other exemplary modifications include those described in U.S. Pat. No.: 5,648,260.

For some antibodies that include an Fc domain, the antibody production system may be designed to synthesize antibodies in which the Fc region is glycosylated. For example, the Fc domain of IgG molecules is glycosylated at asparagine 297 in the CH2 domain. This asparagine is the site for modification with biantennary-type oligosaccharides. This glycosylation participates in effector functions mediated by Fcy receptors and complement Clq (Burton and Woof (1992) Adv. Immunol 51:1-84; Jefferis et al. (1998) Immunol Rev. 163:59-76). The Fc domain can be produced in a mammalian expression system that appropriately glycosylates the residue corresponding to asparagine 297. The Fc domain can also include other eukaryotic post-translational modifications.

Antibodies can also be produced by a transgenic animal. For example, U.S. Pat. No. 5,849,992 describes a method for expressing an antibody in the mammary gland of a transgenic mammal. A transgene is constructed that includes a milk-specific promoter and nucleic acid sequences encoding the antibody of interest, e.g., an antibody described herein, and a signal sequence for secretion. The milk produced by females of such transgenic mammals includes, secreted-therein, the antibody of interest, e.g., an antibody described herein. The antibody can be purified from the milk, or for some applications, used directly.

Antibodies can be modified, e.g., with a moiety that improves its stabilization and/or retention in circulation, e.g., in blood, serum, lymph, bronchoalveolar lavage, or other tissues, e.g., by at least 1.5, 2, 5, 10, or 50 fold.

For example, a VLA-4 binding antibody can be associated with a polymer, e.g., a substantially non-antigenic polymer, such as a polyalkylene oxide or a polyethylene oxide. Suitable polymers will vary substantially by weight. Polymers having molecular number average weights ranging from about 200 to about 35,000 daltons (or about 1,000 to about 15,000, and 2,000 to about 12,500) can be used.

For example, a VLA-4 binding antibody can be conjugated to a water soluble polymer, e.g., a hydrophilic polyvinyl polymer, e.g. polyvinylalcohol or polyvinylpyrrolidone. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene (Pluronics); polymethacrylates; carbomers; branched or unbranched polysaccharides that comprise the saccharide monomers D-mannose, D- and L-galactose, fucose, fructose, D-xylose, L-arabinose, D-glucuronic acid, sialic acid, D-galacturonic acid, D-mannuronic acid (e.g. polymannuronic acid, or alginic acid), D-glucosamine, D-galactosamine, D-glucose and neuraminic acid including homopolysaccharides and heteropolysaccharides such as lactose, amylopectin, starch, hydroxyethyl starch, amylose, dextrane sulfate, dextran, dextrins, glycogen, or the polysaccharide subunit of acid mucopolysaccharides, e.g., hyaluronic acid; polymers of sugar alcohols such as polysorbitol and polymannitol; heparin or heparon.

### Pharmaceutical Compositions

A VLA-4 antagonist, e.g., a VLA-4 binding agent, such as a VLA-4 binding antibody, (e.g., natalizumab) can be formulated as a pharmaceutical composition. Typically, a pharmaceutical composition includes a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible.

A “pharmaceutically acceptable salt” refers to a salt that retains the desired biological activity of the parent compound and does not impart any undesired toxicological effects (see, e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

VLA-4 antagonists, e.g., a VLA-4 binding antibody, e.g., natalizumab, and other agents described herein can be formulated according to standard methods. Exemplary pharmaceutical formulation is described in Gennaro (ed.), Remington: The Science and Practice of Pharmacy, 20.sup.th ed., Lippincott, Williams & Wilkins (2000) (ISBN: 0683306472); Ansel et al., Pharmaceutical Dosage Forms and Drug Delivery Systems, 7.sup.th Ed., Lippincott Williams & Wilkins Publishers (1999) (ISBN: 0683305727); and Kibbe (ed.), Handbook of Pharmaceutical Excipients American Pharmaceutical Association, 3r^{d} ed. (2000) (ISBN: 091733096X).

In one embodiment, a VLA-4 antagonist, e.g., a VLA-4 binding antibody, e.g., natalizumab or another agent (e.g., another antibody) can be formulated with excipient materials, such as sodium chloride, sodium dibasic phosphate heptahydrate, sodium monobasic phosphate, and polysorbate 80. It can be provided, for example, in a buffered solution at a concentration of about 20 mg/ml and can be stored at 2-8° C. Natalizumab can be formulated as described on the manufacturer's label.

Pharmaceutical compositions may also be in a variety of other forms. These include, for example, liquid, semi-solid and solid dosage forms, such as liquid solutions (e.g., injectable and infusible solutions), dispersions or suspensions, tablets, pills, powders, liposomes and suppositories. The preferred form can depend on the intended mode of administration and therapeutic application. Typically compositions for the agents described herein are in the form of injectable or infusible solutions.

Such compositions can be administered by a parenteral mode (e.g., intravenous, subcutaneous, intraperitoneal, or intramuscular injection). The phrases “parenteral administration” and “administered parenterally” as used herein mean modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Pharmaceutical compositions typically must be sterile and stable under the conditions of manufacture and storage. A pharmaceutical composition can also be tested to insure it meets regulatory and industry standards for administration.

The composition can be formulated as a solution, microemulsion, dispersion, liposome, or other ordered structure suitable to high drug concentration. Sterile injectable solutions can be prepared by incorporating an agent described herein in the required amount in an appropriate solvent with one or a combination of ingredients enumerated above, as required, followed by filtered sterilization. Generally, dispersions are prepared by incorporating an agent described herein into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of an agent described herein plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

### Administration

A VLA-4 antagonist, e.g., a VLA-4 binding antibody can be administered to a subject, e.g., a human subject, by a variety of methods. For many applications, the route of administration is one of: intravenous injection or infusion, subcutaneous injection, or intramuscular injection. A VLA-4 binding antibody, such as natalizumab, can be administered as a fixed dose, or in a mg/kg dose, but preferably as a fixed dose. The antibody can be administered parenterally, e.g., intravenously (IV) or subcutaneously (SC).

In some embodiments, a subject is a human subject. In some embodiments, a human subject is an adult subject. In some embodiments, a human subject is a pediatric subject, e.g., a subject who is 18 years of age or younger, 17 years of age or younger, 16 years of age or younger, 15 years of age or younger, 14 years of age or younger, 13 years of age or younger, 12 years of age or younger, 11 years of age or younger, 10 years of age or younger, 9 years of age or younger, 8 years of age or younger, 7 years of age or younger, 6 years of age or younger, 5 years of age or younger, 4 years of age or younger, 3 years of age or younger, 2 years of age or younger, or 1 year of age or younger. In some embodiments, the subject is a human subject diagnosed with sickle cell disease. In some embodiments, the subject is a human subject who is at risk of sickle cell disease, e.g., has one or more risk factors associated with sickle cell disease, e.g., risk factors described herein.

In some embodiments a subject has SCD. In some embodiments, the subject is having or is at elevated risk for an acute vaso-occlusive event. In some embodiments, elevated risk for an acute vaso-occlusive event comprises a 1%, 2%, 3%, 4%, 5%, 6%, 7%, 8%, 9%, 10%, 20%, 30%, 40%, 50%, 60%, 70%, 80%, 90% or more increased risk relative to the general population. In some embodiments, elevated risk for an acute vaso-occlusive event comprises a 1%, 2%, 3%, 4%, 5%, 6%, 7%, 8%, 9%, 10%, 20%, 30%, 40%, 50%, 60%, 70%, 80%, 90% or more increased risk relative to a healthy individual with no vascular complications.

In some embodiments, the antibody, e.g., natalizumab, is administered at a fixed unit dose of between 50-1000 mg IV, e.g., between 100-600 mg IV, e.g., between 150 and 450 mg IV, e.g., between 200 and 400 mg IV, e.g., about 300 mg IV. In some embodiments, the antibody, e.g., natalizumab, is administered at a fixed unit dose of between 100-200 mg IV, e.g., about 150 mg IV. In some embodiments, the antibody, e.g., natalizumab, is administered at a fixed unit dose of between 400-500 mg, e.g., about 450 mg. In some embodiments, the antibody is administered subcutaneously at a dose of between 10 and 500 mg SC. In some embodiments, the antibody is administered subcutaneously at a dose of between 20 and 200 mg SC. In some embodiments, the antibody is administered subcutaneously at a dose of between 37.5 and 112.5 mg SC. In some embodiments, the antibody is administered subcutaneously at a dose of between 50-100 mg SC e.g., about 75 mg SC. In some embodiments, the antibody is administered subcutaneously at a dose of between 10-50 mg SC, e.g., about 37.5 mg SC. In some embodiments, the antibody is administered subcutaneously at a dose of between 100-150 mg SC, e.g., about 112.5 mg SC. It can also be administered in a bolus at a dose of between 1 and 10 mg/kg, e.g., about 6.0, 4.0, 3.0, 2.0, 1.0 mg/kg. In some cases, continuous administration may be indicated, e.g., via a subcutaneous pump.

In some embodiments, the VLA-4 antagonist, e.g., the VLA-4 binding antibody molecule, e.g., natalizumab, is administered at a dose of between 200 and 400 mg. In some embodiments, the VLA-4 antagonist, e.g., the VLA-4 binding antibody molecule, e.g., natalizumab, is administered at a dose of about 300 mg. In some embodiments, the VLA-4 antagonist, e.g., the VLA-4 binding antibody molecule, e.g., natalizumab, is administered at a dose of between 150 and 450 mg. In some embodiments, the VLA-4 antagonist, e.g., the VLA-4 binding antibody molecule, e.g., natalizumab, is administered at a dose of about 150 mg. In some embodiments, the VLA-4 antagonist, e.g., the VLA-4 binding antibody molecule, e.g., natalizumab, is administered at a dose of about 450 mg.

The VLA-4 antagonist, the VLA-4 binding antibody molecule, e.g., natalizumab, can be administered, for example, monthly, e.g., every fourth week, or every 28, 29, 30, or 31 days.

The dose can be chosen to achieve optimal binding of the VLA-4 binding antibody to reticulocytes and/or leukocytes in the subject, and may be at a lower dose than the dose typically given to a subject to treat an inflammatory disorder such as multiple sclerosis (MS) and/or Crohn's disease (CD). For example, the dose can be selected to be less than 300 mg IV. The dose can also be chosen to reduce or avoid production of antibodies against the VLA-4 binding antibody, to achieve greater than 40, 50, 70, 75, or 80% saturation of the α4 subunit, to achieve to less than 80, 70, 60, 50, or 40% saturation of the α4 subunit, or to prevent an increase the level of circulating white blood cells. The dose can also be chosen to maintain optimal levels of hemoglobin.

In some embodiments, the VLA-4 antagonist, e.g., the VLA-4 binding antibody molecule, e.g., natalizumab, is administered to a subject having greater than 60, 70, 75, 76, 77, 78, 79, 80, 81, 82, 63, 84, 85, or 90 g/L hemoglobin in their blood. In some embodiments, the VLA-4 antagonist, e.g., the VLA-4 binding antibody molecule, e.g., natalizumab, is temporarily discontinued if hemoglobin levels in the blood of the subject are lower than 70, 69, 68, 67, 66, 65, 64, 63, 62, 61, or 60 g/L. In some embodiments, the VLA-4 antagonist, e.g., the VLA-4 binding antibody molecule, e.g., natalizumab, is permanently discontinued if hemoglobin levels in the blood of the subject are lower than 60, 59, 58, 57, 56, 55, 54, 53, 52, 51, 50, 45, or 40 g/L. In some embodiments, the VLA-4 antagonist, e.g., the VLA-4 binding antibody molecule, e.g., natalizumab, is discontinued if hemoglobin levels in the blood of the subject decrease by 5, 10, 15, 16, 17, 18, 19, 20, 21, 22, 23, 24, 25, 26, 27, 28, 29, or 30 or more g/L in the blood over a 1, 2, 3, 4, 5, or 6 day or 1, 2, 3, or 4 week period.

In some embodiments, the VLA-4 antagonist, e.g., the VLA-4 binding antibody molecule, e.g., natalizumab, is administered to a subject having elevated reticulocytes. In some embodiments, elevated reticulocytes comprises an increase of 1%, 2%, 3%, 4%, 5%, 6%, 7%, 8%, 9%, 10%, 20%, 30%, 40%, 50%, 60%, 70%, 80%, 90% or more reticulocytes than the general population. In some embodiments, the VLA-4 antagonist, e.g., the VLA-4 binding antibody molecule, e.g., natalizumab, is administered to a subject having greater than 2% reticulocytes in their blood. In some embodiments, the VLA-4 antagonist, e.g., the VLA-4 binding antibody molecule, e.g., natalizumab, is administered to a subject having greater than 5% reticulocytes in their blood. In some embodiments, the VLA-4 antagonist, e.g., the VLA-4 binding antibody molecule, e.g., natalizumab, is administered to a subject having greater than 10% reticulocytes in their blood. In some embodiments, the VLA-4 antagonist, e.g., the VLA-4 binding antibody molecule, e.g., natalizumab, is administered to a subject having greater than 15% reticulocytes in their blood. In some embodiments, the VLA-4 antagonist, e.g., the VLA-4 binding antibody molecule, e.g., natalizumab, is administered to a subject having greater than 20% reticulocytes in their blood. In some embodiments, the VLA-4 antagonist, e.g., the VLA-4 binding antibody molecule, e.g., natalizumab, is administered to a subject having greater than 25% reticulocytes in their blood. In some embodiments, the VLA-4 antagonist, e.g., the VLA-4 binding antibody molecule, e.g., natalizumab, is administered to a subject having greater than 30% reticulocytes in their blood. In some embodiments, the VLA-4 antagonist, e.g., the VLA-4 binding antibody molecule, e.g., natalizumab, is administered to a subject having greater than 40% reticulocytes in their blood.

In certain embodiments, the active agent may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York, 1978.

Pharmaceutical compositions can be administered with medical devices. For example, pharmaceutical compositions can be administered with a needleless hypodermic injection device, such as the devices disclosed in U.S. Pat. No. 5,399,163, 5,383,851, 5,312,335, 5,064,413, 4,941,880, 4,790,824, or 4,596,556. Examples of well-known implants and modules include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Of course, many other such implants, delivery systems, and modules are also known.

Dosage unit form or “fixed dose” as used herein refers to physically discrete units suited as unitary dosages for the subjects to be treated; each unit contains a predetermined quantity of active compound calculated to produce the desired therapeutic effect in association with the required pharmaceutical carrier and optionally in association with the other agent.

A pharmaceutical composition may include a “therapeutically effective amount” of an agent described herein. A therapeutically effective amount of an agent may also vary according to factors such as the disease state, age, sex, and weight of the individual, and the ability of the compound to elicit a desired response in the individual, e.g., amelioration of at least one disorder parameter of sickle cell disease, e.g., a VOC event, the duration of a VOC event, hemoglobin levels, patient-reported fatigue, pain, lactate dehydrogenase, reticulocytes, and/or anemia. A therapeutically effective amount is also one in which any toxic or a detrimental effect of the composition is outweighed by the therapeutically beneficial effects.

In some embodiments, the VLA-4 antagonist is administered as a monotherapy. Methods described herein can also include administering a VLA-4 antagonist in combination with another therapeutic modality, e.g., an additional agent (e.g., a pharmacological agent) or a procedure. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

The VLA-4 antagonist and the at least one additional therapeutic agent can be administered simultaneously, in the same or in separate compositions, or sequentially. For sequential administration, the antagonist can be administered first, and the additional agent can be administered second, or the order of administration can be reversed.

The additional agent is preferably an agent with some degree of therapeutic efficacy in treating sickle cell disease. Such agents may include, but are not limited to a chemotherapeutic agent, e.g., hydroxyurea, RBC transfusions, hematopoietic stem cell transplant, hydration, supplemental oxygen, and/or pain medication.

In some embodiments, hydroxyurea is administered at a dose of between 10 and 40 mg/kg/day. In some embodiments, hydroxyurea is administered at a dose of between 15 and 35 mg/kg/day. In some embodiments, hydroxyurea is administered at a dose of about 15 mg/kg/day. In some embodiments, hydroxyurea is administered at a dose of about 35 mg/kg/day. In some embodiments, hydroxyurea is administered to a subject at a starting dose of about 15 mg/kg/day, and the subject's blood count is monitored periodically. In some embodiments, the subject's blood count is monitored every two weeks. In some embodiments, if the blood count stays in an acceptable range, the dose is increased by 5 mg/kg/day until a dose 35 mg/kg/day is reached if the blood count in an acceptable range. In some embodiments, the dose is increased every 12 weeks. In some embodiments, if the blood count is between an acceptable range and a toxic range, the dose is not increased. In some embodiments, if the blood count is in a toxic range, hydroxyurea is discontinued until hematological recovery is achieved. In some embodiments, once hematological recovery is achieved, the dose is reduced by 2.5 mg/kg/day and increased by 2.5 mg/kg/day until a stable dose is achieved. In some embodiments, the dose is increased every 12 weeks. In some embodiments, a blood count in an acceptable range comprises neutrophils greater than or equal to 2500 cells/mm³, platelets greater than or equal to 95,000/mm³, hemoglobin greater than 5.3 g/dl, and reticulocytes greater than or equal to 95,000/mm³ if the hemoglobin concentration is less than 9 g/dl. In some embodiments, a blood count in a toxic range comprises neutrophils less than 2000 cells/mm³, platelets less than 80,000/mm³, hemoglobin less than 4.5 g/dl, and reticulocytes less than 80,000/mm³ if the hemoglobin concentration is less than 9 g/dl.

In some embodiments, a second agent is a RBC transfusion. In some embodiments, a RBC transfusion is administered at a dose of 0.5, 1, 1.5, 2, 3 or more pints of RBCs.

In some embodiments, a second agent is a pain medication. Exemplary pain medications include but are not limited to ibuprofen, aspirin, naproxen sodium, acetaminophen, diclofenac sodium, etodolac, fenoprofen, flurbiprofen, indomethacin, ketorolac tromethamine, nabumetone, naproxen, oxaprozin piroxicam, sulindac, darvocet, percocet, percodan, vicodin, oxycontin, dilaudid, and/or demerol.

### Methods of Treatment

The methods of treatment described herein include administering to a subject suffering from sickle cell disease an effective amount of a VLA-4 antagonist. Treating includes administering an amount effective to alleviate, relieve, alter, remedy, ameliorate, improve or affect the disorder, the symptoms of the disorder or the predisposition toward the disorder. The treatment may also delay onset, e.g., prevent onset, or prevent deterioration of a disease or condition.

SCD is a congenital disease caused by the inheritance of a mutant β-globin allele (G1u6Val) resulting in abnormal hemoglobin, which is the oxygen carrying molecule in red blood cells (RBCs). The sickle mutation can be inherited on both alleles, producing homozygous genotype Hb SS SCD, the most common form of SCD. The sickle mutation can also be inherited in trans with the other allele specifying a dysfunctional 3-globin (genotype Hb S-β(+) thalassemia), absent β-globin (genotype Hb S-α(0) thalassemia), or a different 3-globin point mutation, such as Hemoglobin C (leading to Hb SC disease). In general, genotypes Hb SS and Hb S-β(0) thalassemia are clinically similar and are the most severe forms of SCD, as only sickle β-globin is expressed in each case.

The pathophysiology of SCD is initiated by the propensity of the abnormal sickle hemoglobin to polymerize under low oxygen tension. This distorts RBCs into the sickle shapes and renders them increasingly prone to hemolysis compared to normal RBCs. Sickle RBCs are also more adhesive than normal RBCs, capable of adhering to the endothelial surface, plasma proteins, and other blood cells (Hebbel RP et al, Microcirculation 11(2):129-512004). SCD pathophysiology also involves increased leukocyte and platelet adhesion, as well as increased inflammation, hypercoagulability, oxidative stress, and increased cell activation that contribute to the overall hyperadhesive phenotype of the disease (Frenette PS et al. J Clin Invest 117(4):850-8 2007; Hebbel).

Symptoms of SCD include, e.g., hemolytic anemia, vaso-occlusive events (VOC), early mortality, hand-foot syndrome, gallstones, stroke, silent stroke, gallstones, splenic sequestration, hyposplenism, acute chest syndrome, acute papillary necrosis, aplastic crisis, haemolytic crisis, dactylitis, background retinopathy, proliferative retinopathy, vitreous haemorrhages and retinal detachments, vision loss, intrauterine growth retardation, spontaneous abortion, pre-eclampsia, frequent infections, intravascular hemolysis, vasculopathic complications, pulmonary hypertension, heart failure, syncope, leg ulcers, priapism, infarction of the penis, osteomyelitis, opioid tolerance, organ damage, fatigue, irritability, dizziness, delayed puberty, slowed growth, paleness of the skin, jaundice, shortness of breath, cognitive dysfunction, chronic pain, chronic renal failure, proteinuria, haematuria, and/or failure of multiple organ systems, including renal, hepatic, cerebral, cardiovascular, pulmonary, and avascular necrosis-related bone and joint disease.

SCD may be determined by any available method. In some cases, hemoglobin S is detected in the blood of a subject. In some cases, blood of a subject is examined under a microscope to detect red blood cells with a sickled shape. In some cases, blood of a subject is tested for anemia. In some cases, DNA of a subject is tested for one or two copies of the mutant β-globin allele. In some cases, DNA of a subject is obtained from amniotic fluid of an unborn child.

Therapies used to treat SCD can include, e.g., hematopoietic stem cell transplant (HSCT) from an appropriate immunologically matched donor, either a sibling, a haploidentical relative, or cord blood source, hydroxyurea, RBC transfusions and/or supportive care including hydration, supplemental oxygen, pain medication, and aggressive monitoring and early treatment of associated complications.

In other disorders, e.g., multiple sclerosis (MS), natalizumab inhibits lymphocyte adhesion and extravasation, preventing the excessive recruitment of autoreactive immune cells into tissues and thereby preventing the formation of inflammatory lesions that are the hallmark of MS. Without wishing to be bound by any particular theory, in SCD, blockade of α4 integrins by natalizumab is hypothesized to block reticulocyte and lymphocyte adhesion thereby preventing vaso-occlusion and possibly limiting the consequences of endovascular inflammation.

Reticulocytes express α4β1 (VLA-4) on their surface, while leukocytes (lymphocytes and monocytes but not neutrophils) express α4β1 as well as α4β7. Reticulocytes are anucleate cells that are the initial erythrocyte precursor released by the bone marrow into circulation with their production being increased in response to anemia. Due to chronic hemolytic anemia, reticulocyte counts are up to 30 times higher in patients with SCD than in non-anemic individuals. Reticulocytes mature in the periphery over 3 to 5 days into mature erythrocytes, during which time reticulocyte surface VLA-4 decreases such that mature erythrocytes do not express VLA-4 on their cell surface. The increased inflammatory and oxidative states inherent to SCD are known to result in converting reticulocyte VLA-4 into its active binding conformation, as opposed to the inactive conformation normally found in non SCD individuals (Brittain J E et al. J Biol Chem 279(41):42393-402 2004).

In SCD, VLA-4 mediates the adhesion of reticulocytes to the vascular endothelium via binding to VCAM-1 on the endothelial surface. In addition to increased circulating VLA-4-positive reticulocytes, endothelial VCAM-1 is also markedly increased in individuals with SCD compared to individuals without SCD. VLA-4 on SCD blood cells can also mediate cell to cell adhesion either directly or indirectly through bridging molecules such as fibronectin (Brittain J E et al. Transfus Clin Biol 15(1-2):19-22 2008). Lymphocyte α4 integrins can also bind mucosal address in cell adhesion molecule-1 (MAdCAM-1), in addition to VCAM-1, osteopontin, and fibronectin. Blockade of the VLA-4 interaction with VCAM-1 has been shown to reduce sickle blood cell adhesion in ex vivo adhesion models under fluid flow conditions (See Example 1), as well as limit cell adhesion and vaso occlusion in vivo in murine intravital microscopy experimental systems (Belcher J D et al. Am J Physiol Heart Circ Physiol 288(6):H2715-25 2005).

Without wishing to be bound by any particular theory, due to binding to VLA-4 on reticulocytes, treatment with natalizumab has the potential to result in decreased adhesion of reticulocytes to the endothelium, as well as to limit the formation of cell:cell aggregates that lead to vaso-occlusion. Natalizumab blockade of lymphocyte adhesion may also limit the formation of heterocellular vaso-occlusive aggregates and may decrease inflammation, as well as the consequences of ischemia:reperfusion injury. It is expected that blockade of α4 integrins by natalizumab may also speed the transit time of sickle RBC through the vasculature, thus limiting the propensity for deoxygenation-induced hemoglobin polymerization and reducing hemolysis. Natalizumab binding may therefore interrupt the cycle of adhesion, deoxygenation, and inflammation that drives disease pathogenesis, potentially resulting in reduced vaso-occlusion, improved RBC survival, normalized blood flow, and decreased inflammation. The reduction in adhesion and hemolysis may limit the inflammation, oxidation, and endothelial activation that further subserve disease consequences. Without wishing to be bound by any particular theory, clinically, natalizumab is expected to result in therapeutic benefits including decreased VOC rate, improved hemoglobin, decreased fatigue, decreased pain, and decreased opiate use. By limiting these consequences, long-term benefits of natalizumab administration in subjects with SCD may be reduced end organ damage, improved morbidity, and decreased mortality.

SCD improvement comprises an improvement in a subject at a second time point relative to a first timepoint. In some embodiments, the first time point is 1, 2, 3 weeks, 1, 2, 3, 4, 5, 6, 7, 8, 9, 10, 11 months, or 1, 2, 3, 4, 5 or more years prior to the second time point. In some embodiments, the first timepoint is before initiation of treatment with a VLA-4 antagonist. In some embodiments, the second time point is after initiation of treatment with a VLA-4 antagonist. In some embodiments, the first timepoint is before initiation of treatment with a VLA-4 antagonist, and the second time point is after initiation of treatment with a VLA-4 antagonist.

Standard tests for SCD improvement include, e.g., a decrease in VOC events, a decreased duration of a VOC event, an increase in hemoglobin levels, an improvement of patient-reported fatigue, a decrease in pain, a decrease in lactate dehydrogenase, a decrease in reticulocytes, an increase in red blood cell (RBC) levels, and/or a decrease in anemia.

In some embodiments, SCD improvement comprises a decrease in VOC events. In some embodiments, a decrease in VOC events comprises a reduction in the total number of annualized VOC events. In some embodiments, a decrease in VOC events is a reduction to 1, 2, 3, 4, or 5 VOC events in the year after receiving the VLA-4 antagonist compared to the year prior to receiving the VLA-4 antagonist.

In some embodiments, SCD improvement comprises a decrease in the duration of VOC events. In some embodiments, the duration of a VOC event at a second time point is reduced by 1%, 2%, 3%, 4%, 5%, 6%, 7%, 8%, 9%, 10%, 20%, 30%, 40%, 50%, 60%, 70%, 80%, 90% or more relative to the duration of a VOC event at a first time point. In some embodiments, the average duration of a VOC event at a second time point is reduced by 1%, 2%, 3%, 4%, 5%, 6%, 7%, 8%, 9%, 10%, 20%, 30%, 40%, 50%, 60%, 70%, 80%, 90% or more relative to an average duration of a VOC event at a first time point.

In some embodiments, SCD improvement comprises a decrease in patient reported fatigue. In some embodiments, patient reported fatigue at a second time point is reduced by 1%, 2%, 3%, 4%, 5%, 6%, 7%, 8%, 9%, 10%, 20%, 30%, 40%, 50%, 60%, 70%, 80%, 90% or more relative to patient reported fatigue at a first time point. In some embodiments, fatigue is measured by an objective fatigue score.

In some embodiments, SCD improvement comprises a decrease in reticulocytes. In some embodiments, reticulocyte levels at a second time point are decreased by 1%, 2%, 3%, 4%, 5%, 6%, 7%, 8%, 9%, 10%, 20%, 30%, 40%, 50%, 60%, 70%, 80%, 90% or more relative to reticulocyte levels at a first time point.

In some embodiments, SCD improvement comprises an increase in RBC levels. In some embodiments, hemoglobin levels at a second time point are increased by 1%, 2%, 3%, 4%, 5%, 6%, 7%, 8%, 9%, 10%, 20%, 30%, 40%, 50%, 60%, 70%, 80%, 90% or more relative to hemoglobin levels at a first time point.

In some embodiments, SCD improvement comprises an increase in hemoglobin levels. In some embodiments, hemoglobin levels at a second time point are increased by 1%, 2%, 3%, 4%, 5%, 6%, 7%, 8%, 9%, 10%, 20%, 30%, 40%, 50%, 60%, 70%, 80%, 90% or more relative to hemoglobin levels at a first time point.

In some embodiments, SCD improvement comprises a decrease in patient reported pain. In some embodiments, patient reported pain at a second time point is reduced by 1%, 2%, 3%, 4%, 5%, 6%, 7%, 8%, 9%, 10%, 20%, 30%, 40%, 50%, 60%, 70%, 80%, 90% or more relative to patient reported pain at a first time point. In some embodiments, pain is measured by a 10 point score. In some embodiments, patient reported pain at a second time point is reduced by 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3, 4, 5, 6, 7 or more points on a 10 point pain scale relative to patient reported pain at a first time point.

In some embodiments, SCD improvement comprises a decrease in anemia. In some embodiments, anemia at a second time point is decreased by 1%, 2%, 3%, 4%, 5%, 6%, 7%, 8%, 9%, 10%, 20%, 30%, 40%, 50%, 60%, 70%, 80%, 90% or more relative to anemia at a first time point.

### Method for Detecting VLA-4

The invention described herein encompasses the recognition that expression of VLA-4 on cells can be detected by adherence of cells to VCAM-1.

In some embodiments, VCAM-1 is immobilized on a surface. In some embodiments, the surface is planar. In some embodiments, the surface is a channel or cylindrical surface through which liquid can flow. In one exemplary embodiment, the channel is a commercial well-plate micro-fluidic flow adhesion system. In some embodiments, immobilization of a channel is performed by perfusion of 0.001-1 mg/mL VCAM-1 at 0.1-10 dynes/cm² for 1-30 minutes. In some embodiments, immobilization of a channel is performed by perfusion of 0.01-05 mg/mL VCAM-1 at 1-5 dynes/cm2 for 2-10 minutes. In one exemplary embodiment, immobilization of a channel is performed by perfusion of 0.02 mg/mL VCAM-1 at 2 dynes/cm² for 5 minutes.

In some embodiments, the surface is perfused with a blocking agent to remove unbound VCAM-1. In some embodiments, a blocking agent is BSA. In some embodiments, blocking of a channel is performed by perfusion of 0.01%-10% BSA at 0.1-10 dynes/cm² for 1-30 minutes. In some embodiments, blocking of a channel is performed by perfusion of 0.1%-1% BSA at 0.2-1 dynes/cm² for 5-15 minutes. In one exemplary embodiment, blocking of a channel is performed by perfusion of 0.5% BSA at 5 dynes/cm² for 10 minutes.

In some embodiments, blood samples from a subject are contacted with a VLA-4 antagonist, e.g., the VLA-4 binding antibody molecule, e.g., natalizumab, before being contacted with the channel. In some embodiments, the blood is pretreated with between 0.00001 μg/mL and 1 mg/ml of the VLA-4 antagonist, e.g., the VLA-4 binding antibody molecule, e.g., natalizumab. In some embodiments, the blood is pretreated with between 0.0001 and 200 μg/mL of the VLA-4 antagonist, e.g., the VLA-4 binding antibody molecule, e.g., natalizumab. In some embodiments, the blood is pretreated with between 0.001 and 20 μg/mL of the VLA-4 antagonist, e.g., the VLA-4 binding antibody molecule, e.g., natalizumab. In some embodiments, the blood is pretreated for between 1 and 60 minutes. In some embodiments, the blood is pretreated for about 30 minutes.

In some embodiments, the blood is diluted in a buffer. In one exemplary embodiment, blood is diluted 1:2 in HBSS containing 1 mM Ca2+ 1mM Mg2+ and/or 1 mM Mn2+.

In some embodiments, blood samples from subjects are then contacted with the channel under flow conditions. In some embodiments, cells are adhered for 1-10 minutes at 0.1-10 dyne/cm². In one exemplary embodiment, cells are adhered for 1 minute at 1 dyne/cm². In one exemplary embodiment, cells are adhered for 5 minutes at 1 dyne/cm².

Standard protocols for fixing and staining the cells can be used to detect binding of blood cells to the channel. In some embodiments, blood cells are fixed to the channel using a fixative, e.g., formalin or formaldehyde. In one exemplary embodiment, blood cells are fixed with 4% formalin. In one exemplary embodiment, blood cells are blocked in an Fc blocking reagent in HBSS-BSA and stained with anti-CD71 antibody diluted1:20 dilution overnight at 4 ° C. In one exemplary embodiment, adhered stained cells blood cells were washed with 1×PBS and stained with DAPI. In one exemplary embodiment, images were acquired with a high resolution CCD camera, in the center of each channel, within the viewing window.

In some embodiments, adherence of blood to the channel indicates the presence of VLA-4 on the surface of the blood cells. In some embodiments, adherence of blood to the channel in the absence of natalizumab but showing reduced adherence in the presence of natalizumab indicates that VLA-4 on the cell surface binds natalizumab. In some embodiments, a 1%, 2%, 3%, 4%, 5%, 6%, 7%, 8%, 9%, 10%, 20%, 30%, 40%, 50%, 60%, 70%, 80%, 90% or more reduction of blood cell binding in the presence of natalizumab relative to the absence of natalizumab indicates reduced adherence of blood cells in the presence of natalizumab.

Methods of Evaluating Samples and/or Subjects

As used herein, methods of evaluating or analyzing a subject or biological sample from a subject include one or more of performing the analysis of the sample, requesting analysis of the sample, requesting results from analysis of the sample, or receiving the results from analysis of the sample. Generally herein, determination (or determining), analysis or evaluation (or evaluating) can include one or both of performing the underlying method or receiving data from another who has performed the underlying method.

The analysis or evaluation requires a transformation of material, e.g., biological material or assay components. For example, a biological sample (e.g., whole blood or plasma) can be evaluated for the presence of VLA-4. The evaluation can be performed before, after or at the same time the patient is receiving treatment, such as for SCD. The evaluation is based, at least in part, on analysis of a sample from the subject, e.g., a blood, plasma, or serum sample. In one embodiment, the biological sample obtained from a patient comprises blood. In some embodiments, blood comprises white blood cells (WBCs). In some embodiments, blood comprises leukocytes. In some embodiments, blood comprises red blood cells (RBCs). Methods for obtaining blood fractions are known in the art.

The presence of VLA-4 can be determined by contact the sample with a specific binding agent, e.g., VCAM-1. In one embodiment, the sample is analyzed for the number of blood cells expressing VLA-4 in the sample, e.g., by a method described herein. For example, blood cells in the sample can be contacted with a channel coated with VCAM-1 under shear stress conditions and the number of cells adhered to the channel can be assayed. In one embodiment, reticulocytes are subject to the adhesion method described herein after exposure to the VLA-4 antagonist, e.g., the VLA-4 binding antibody molecule, e.g., natalizumab, to identify whether of the VLA-4 antagonist, e.g., the VLA-4 binding antibody molecule, e.g., natalizumab, is likely to decrease adhesiveness of the reticulocytes.

The biological sample can be removed from the patient and analyzed. In some embodiments, the sample, e.g., plasma or whole blood sample can be stored prior to testing for the presence of VLA-4 and/or responsiveness to natalizumab. The sample, e.g., the sample containing VLA-4, can be stored for 1-21 days, e.g., 1-14 days or 1-7 days or longer (e.g., one day, two days, three days, five days, seven days, ten days, 14 days, 21 days or longer) for one to six weeks, e.g., one to three weeks or one to two weeks or longer (e.g., up to one week, up to two weeks, up to three weeks, up to six weeks, or longer); or for one to six months, e.g., one to three months or one to two months or longer (e.g., up to one month, up to two months, up to three months, up to six months or longer). The sample can be stored, for example, frozen (e.g., at −80° C. to −20° C.), at 2-8° C., at ambient temperature (18° C.-25° C.) or warmer, e.g., at 37° C.

At least one or both of determining a patient's status (e.g., a candidate for treatment with a VLA-4 antagonist, e.g., a VLA-4 binding antibody molecule, e.g., natalizumab) and determining if the status has a preselected relationship with a reference standard, includes one or more of analyzing a sample, requesting analysis of the sample, requesting results from analysis of the sample, or receiving the results from analysis of the sample. (Generally, analysis can include one or both of performing the underlying method (e.g., a method described herein, e.g., an immunoassay under flow conditions) or receiving data from another who has performed the underlying method.)

### Kits

A VLA-4 antagonist described herein may be provided in a kit. The kit includes a VLA-4 antagonist described herein and, optionally, a container, a pharmaceutically acceptable carrier and/or informational material. The informational material can be descriptive, instructional, marketing or other material that relates to the methods described herein and/or the use of the u4 antagonist for the methods described herein.

The informational material of the kits is not limited in its form. In one embodiment, the informational material can include information about production of the VLA-4 antagonist, physical properties of the α4 antagonist, concentration, date of expiration, batch or production site information, and so forth. In one embodiment, the informational material relates to methods for administering the VLA-4 antagonist, e.g., by a route of administration described herein and/or at a dose described herein.

In one embodiment, the informational material can include instructions to administer a VLA-4 antagonist described herein in a suitable manner to perform the methods described herein, e.g., in a suitable dose, dosage form, or mode of administration (e.g., a dose, dosage form, or mode of administration described herein). In another embodiment, the informational material can include instructions to administer a VLA-4 antagonist to a suitable subject, e.g., a human, e.g., a human having or at risk for sickle cell disease.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about a VLA-4 antagonist described herein and/or its use in the methods described herein. The informational material can also be provided in any combination of formats.

In addition to an α4 antagonist, the composition of the kit can include other ingredients, such as a surfactant, a lyoprotectant or stabilizer, an antioxidant, an antibacterial agent, a bulking agent, a chelating agent, an inert gas, a tonicity agent and/or a viscosity agent, a solvent or buffer, a stabilizer, a preservative, a pharmaceutically acceptable carrier and/or a second agent for treating a condition or disorder described herein. In some embodiments, the second agent is hydroxyurea. Alternatively, the other ingredients can be included in the kit, but in different compositions or containers than a VLA-4 antagonist described herein.

In some embodiments, a component of the kit is stored in a sealed vial, e.g., with a rubber or silicone closure (e.g., a polybutadiene or polyisoprene closure). In some embodiments, a component of the kit is stored under inert conditions (e.g., under Nitrogen or another inert gas such as Argon). In some embodiments, a component of the kit is stored under anhydrous conditions (e.g., with a desiccant). In some embodiments, a component of the kit is stored in a light blocking container such as an amber vial.

A VLA-4 antagonist described herein can be provided in any form, e.g., liquid, frozen, dried or lyophilized form. It is preferred that a composition including the VLA-4 antagonist described herein be substantially pure and/or sterile. When a VLA-4 antagonist described herein such as natalizumab is provided in a liquid solution, the liquid solution preferably is an aqueous solution, with a sterile aqueous solution being preferred. In one embodiment, the VLA-4 antagonist is supplied with a diluents or instructions for dilution. The diluent can include for example, a salt or saline solution, e.g., a sodium chloride solution having a pH between 6 and 9, lactated Ringer's injection solution, D5W, or PLASMA-LYTE A Injection pH 7.4® (Baxter, Deerfield, Ill.).

The kit can include one or more containers for the composition containing a VLA-4 antagonist described herein. In some embodiments, the kit contains separate containers, dividers or compartments for the composition and informational material. For example, the composition can be contained in a bottle, vial, IV admixture bag, IV infusion set, piggyback set or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. The containers of the kits can be air tight, waterproof (e.g., impermeable to changes in moisture or evaporation), and/or light-tight.

The invention is further illustrated by the following examples, which should not be construed as limiting.

### EXAMPLES

### Example 1

### VLA-4 Mediated Inhibition of Erythrocyte Adhesion: A Potential Application of Natalizumab in Patients with Sickle Cell Disease

Cell surface adhesion molecules play a crucial role in orchestrating key events of inflammation in health and disease. Among these molecules, VLA-4 has been demonstrated to be an integrin in various cell types, blockade of which by monoclonal antibodies is believed to be beneficial in the treatment of disease conditions with an underlying pathological inflammation. The results presented herein evaluated the role of blocking VLA-4 in blood cells with natalizumab to mitigate pathologic vascular adhesion/obstruction in sickle cell disease (SCD). Systematic titration studies with natalizumab revealed the presence of saturable levels of VLA-4 on the surface of both leukocytes and reticulocytes from blood of sickle-cell patients. These cells exhibited adhesion to immobilized VCAM-1 under conditions of shear stress mimicking the endogenous microcirculation. Additionally, the VLA-4 dependent adhesion of leukocytes and reticulocytes to VCAM-1 was blocked by natalizumab in a dose dependent fashion, which correlated with cell surface saturation binding.

Vaso-occlusion and hemolytic anemia are clinical hallmarks of sickle cell disease (SCD). The pathophysiology of vaso-occlusive episodes is multifactorial, including polymerization of hemoglobin leading to red blood cell (RBC) sickling (Eaton W A et al. Blood 70(5):1245-66 1987). Hemoglobin polymerization distorts sickle RBC, and renders them more prone to hemolyze in circulation. In addition to the phenomenon of sickling, these erythrocytes (SSRBCs) have a greater propensity to adhere to the vasculature and this property has been implicated as a key component of the pathophysiology of the disease (Hebbel R P et al. N Engl J Med 302(18):992-5 1980; Hebbel R P et al. J Clin Invest 65(1):154-60 1980). The adhesive properties of SSRBCs may be assessed clinically to predict risk of vaso-occlusive complications, guide therapy, or monitor response to therapy in patients with SCD (Brittain J E et al. J Clin Invest 107(12):1555-62 2001; Hines P C et al. Blood 101(8):3281-7 2003; Lee S P et al. Blood 92(8):2951-8 1998; Wagner M C et al. J Lab Clin Med 144(5): p. 260-7; discussion 227-8 2004). The initiation and propagation of vaso-occlusion results from processes that impair blood flow through the microvasculature, and these processes also promote hemolysis. In particular, it is thought that adhesive interactions promote the formation of blood flow-obstructing heterocellular aggregates that induce ischemic tissue damage and slow the transit of RBCs through the vasculature, promoting sickle hemoglobin polymerization and increased hemolysis. The obstruction of blood flow and resulting hypoxia triggers an inflammatory response exacerbating vaso-occlusion by stimulating the endothelium to become a more adhesive substrate (Setty B N et al. Blood 88(6):2311-20 1996; Kaul D K et al. J Clin Invest 106(3):411-20 2000). This is believed to act as a nidus and further activate circulating leukocytes which then participate in the formation of additional cell-cell aggregates, of which the RBC is a key component, and support additional interactions with endothelial cells. These discrete yet, highly related phenomena, are thought to pave the way to the precipitation of vaso-occulusive crisis and hemolysis in SCD patients, the leading causes of morbidity and mortality.

Many of the vascular interactions between various cell types are mediated through a plethora of cell surface adhesion molecules that are expressed in the inflammatory microenvironment. Numerous receptor/ligand couples mediating SSRBC adhesion have been confirmed. Very late antigen-4 (VLA-4) or α4β1 integrin is a RBC adhesion molecules that supports interactions between SSRBCs and endothelial VCAM (Joneckis C C et al. Blood 82(12):3548-55 1993; Swerlick R A et al. Blood 82(6):1891-9 1993). VLA-4 is highly expressed on leukocytes and is reported to be the only integrin expressed on immature RBCs that are found in increased numbers in the peripheral blood of SCD patients (Hemler M E et al. J Biol Chem 262(24):11478-85 1987; Joneckis C C et al.; Swerlick R A et al.). SSRBCs and leukocytes have been shown to adhere directly to activated endothelium and this interaction can be reversed by antibodies to the VLA-4 ligand (Swerlick R A et al.; Belcher J D et al. Am J Physiol Heart Circ Physiol 288(6):H2715-25 2005).

Natalizumab is a FDA-approved, humanized, monoclonal antibody against the α4 subunit of the integrin VLA-4 used for the treatment of multiple sclerosis and Crohn's disease. Natalizumab's mechanism of action is believed to involve the prevention of immune cell migration across the blood vessel wall to reach affected organs by inhibiting the interaction between VLA-4 on the leukocyte and endothelial VCAM-1.

The data presented herein investigates the effect of natalizumab on erythrocyte and leukocyte adhesion in whole blood and describes the patient-to-patient variability in adhesive response to natalizumab. The results presented herein describe a useful bioassay to predict patients likely to respond to natalizumab for inclusion in clinical studies, to select the appropriate anti-adhesive therapy for an individual patient, and to follow individual patient response to natalizumab in future clinical studies or patient therapy.

### Materials and Methods

### Reagents

Natalizumab and biotinylated natalizumab were provided by Biogen Idec. Anti CD45, anti-CD29, PE isotype control, Fc receptor blocking solution, and erythrocyte lysis buffer were purchased from eBioscience (San Diego, Calif.); anti-CD71 from Abcam (Cambridge, Mass.). Control IgG4 antibody was from Millipore (Billerica, Mass.). Secondary antibody (PE labeled anti-human IgG4) was purchased from Southern Biotech (Birmingham, Ala.). Recombinant human VCAM-1 (rhVCAM-1) was purchased from R&D Systems (Minneapolis, Minn.). Diamidino-2-phenylindole (DAPI) was obtained from Sigma-Aldrich (St. Louis, Mo.).

### Patients and Blood Donors

The study described herein was performed at two institutions. One study was largely done using pediatric donors between ages 0.83 and 18 years. Informed parental consent, or patient assent, was obtained. Additional blood samples used to compare SCD donors and healthy controls were obtained from adult donors greater than 18 years of age using approved protocols.

### Blood Preparation

Blood was drawn by venipuncture and centrifuged at 150 g for 15 minutes at 25° C. to isolate white blood cells (WBCs). The buffy coat was separated from the SSRBC pellet and platelet rich plasma. All buffers were pre-warmed to 37° C. prior to use. For flow adhesion assays, WBCs (2.5×105 cells/mL) and whole blood (1:2 dilution) were diluted in Hanks balanced salt solution (HBSS) supplemented with 1 mM calcium and 1 mM magnesium, and in some cases 1mM manganese, pH=7.4. For the adult donor study, leukocytes from blood samples were isolated using the CD45 beads and whole blood columns (Miltenyi Biotech). Isolated leukocytes were resuspended in HBSS for adhesion assays, similar to whole blood. For some of the VLA-4 saturation studies, RBCs were separated from the leukocytes in whole blood using the same CD45 bead based system, where the unlabeled flowthrough cells were collected as the RBC enriched fraction.

### Flow Adhesion Assay

Flow adhesion assays were performed with a commercial well-plate micro-fluidic flow adhesion system, Bioflux 1000Z (Fluxion, San Francisco, Calif.). Coating of the microfluidic channels was performed by perfusion with 0.02 mg/mL of VCAM-1 at 2 dynes/cm2 for 5 minutes followed by incubation at 37° C. for 1 hr. Channels were then perfused with HBSS (37° C.) containing 0.5% bovine serum albumin at 5 dynes/cm2 for 10 minutes to remove any unbound VCAM-1 substrate. Flow conditions for adhesion assays were performed using either constant or pulsatile flow (1.67 Hz), as indicated at a shear stress of 1.0 dyne/cm2. For VLA-4 inhibition assays, leukocytes or whole blood diluted 1:2 in HBSS containing 1 mM Ca2+ 1 mM Mg2+ and/or 1 mM Mn2+, and pre-treated with vehicle or varying concentrations of natalizumab for 30 minutes and perfused through micro-fluidic channels. Whole blood cells and isolated leukocytes were allowed to adhere for 1 and 5 minutes at 1 dyne/cm2 for isolated, respectively. Following cell adhesion, adherent cells were fixed with 4% formalin, blocked using an Fc blocking reagent in HBSS-BSA and stained with anti-CD71 antibody (1:20 dilution) overnight at 4° C. Adhered stained cells were washed with 1×PBS and stained with DAPI. Images were acquired with a high resolution CCD camera, in the center of each channel, within the viewing window. Montage imaging software (Molecular Devices, Downington, Pa.) was used to analyze images. Bright field and fluorescent images were overlaid and each cell type was quantified manually. Adherent cells were scored using the following criteria: nucleated leukocytes (DAPI+/CD71−) or (DAPI+/CD71+), reticulocytes (DAPI−/ CD71+), and mature erythrocytes (DAPI−/CD71−).

### Staining for VLA-4 in SCD Leukocytes and Reticulocytes

Leukocyte VLA-4: Whole blood was first incubated with an Fc blocking solution for 20 minutes on ice followed with either natalizumab or IgG4 at 10 μg/mL and APC labeled anti-CD45 antibody for 30 minutes at 4° C. Post incubation, erythrocytes were lysed using an RBC lysis buffer and the resulting leukocytes were then incubated with secondary anti-IgG4 PE labeled antibody. After 30 minutes at 4° C., cells were centrifuged again and resuspended in PBS for flow cytometry analysis.

Reticulocyte staining: 2.5 μL of whole blood was diluted with 100 μL of PBS and incubated with natalizumab or IgG4 at 10 μg/mL or anti-CD29 or its isotype control for 30 minutes at 4 ° C. Cells were then centrifuged at 1500 rpm for 10 minutes (while the centrifuge brake was deactivated), and the pellet was resuspended in 100 μL of PBS containing a 1:10 dilution of secondary anti-IgG4 PE labeled antibody and 5 μL of eFluor-710 labeled anti-CD235a antibody. After 30 minutes at 4° C., cells were centrifuged again and resuspended in 500 μL of BD-Retic reagent (thiazole orange) for 30 minutes at room temperature, after which they were subjected to flow cytometry.

Flow cytometry was carried out using the BD FACS Canto II and data analysis was performed with the FACS Diva software. Data for leukocytes was acquired by gating based on forward and side scatter properties and CD45 staining of mononuclear cells. Natalizumab and IgG4 staining of the CD45 positive mononuclear cells were then determined. For erythrocytes, cells were gated for CD235a positive staining and examined for thiazole orange stained reticulocytes. Reticulocyte staining for VLA-4 with either natalizumab or an anti CD29 antibody was then determined and compared to IgG4 or isotype matched control antibody.

For each determination, the mean fluorescence intensity (MFI) was recorded. MFI values for natalizumab staining were determined by subtracting the MFI of IgG4 from the MFI for each natalizumab concentration. In order to calculate the average binding to leukocytes or reticulocytes from multiple donor samples, the percent binding at each natalizumab concentration was calculated by setting the maximum MFI at saturation to 100%.

### Statistical Analysis

Patient demographics and outcome measures were assessed with descriptive statistics, including means with standard error of means for skewed continuous variables. A Shapiro-Wilk test was used to determine whether data was normally distributed. Wilcoxon Signed Ranks test and paired t-test were used to determine the effects of natalizumab on baseline adhesion to VCAM-1. Pearson's correlation coefficients were calculated between adhesion and clinical hematological values. SPSS version 21.0 (IBM Inc., Chicago Ill. 2012) was used for statistical analysis. All tests are two-tailed test. P-value<0.05 was considered statistically significant.

### Results

### Natalizumab Recognized VLA-4 Surface Expression in SCD Reticulocytes and Leukocytes.

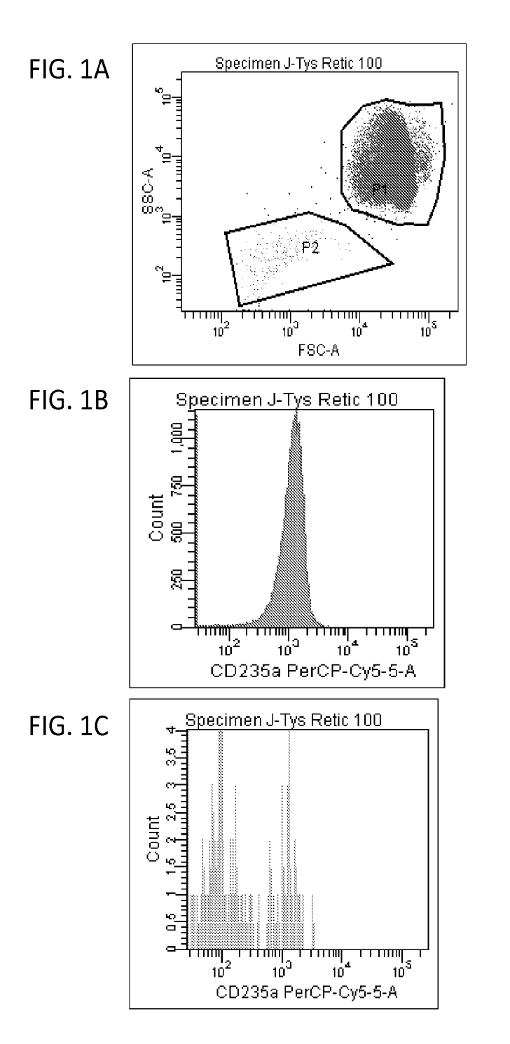

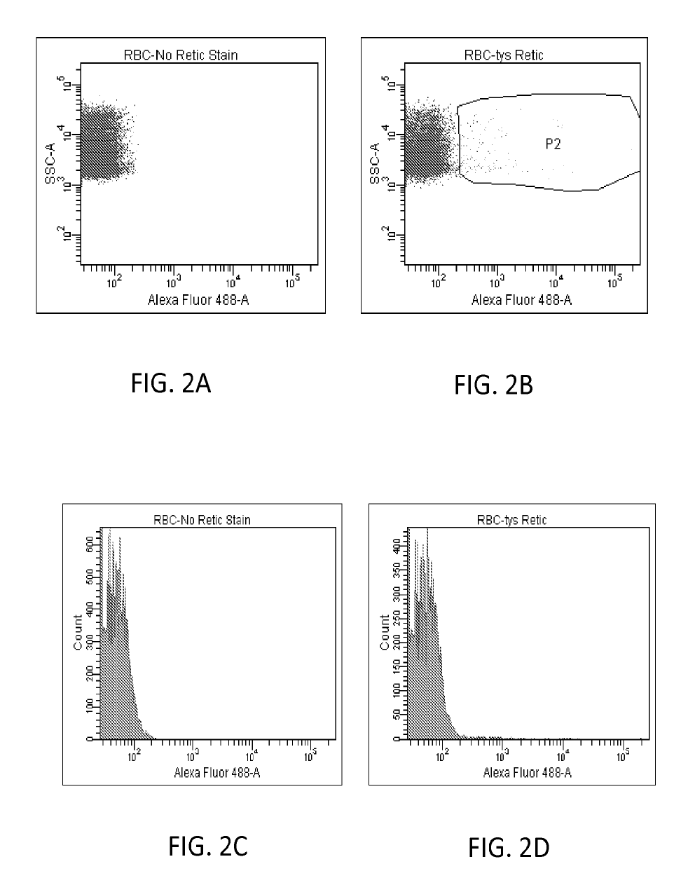

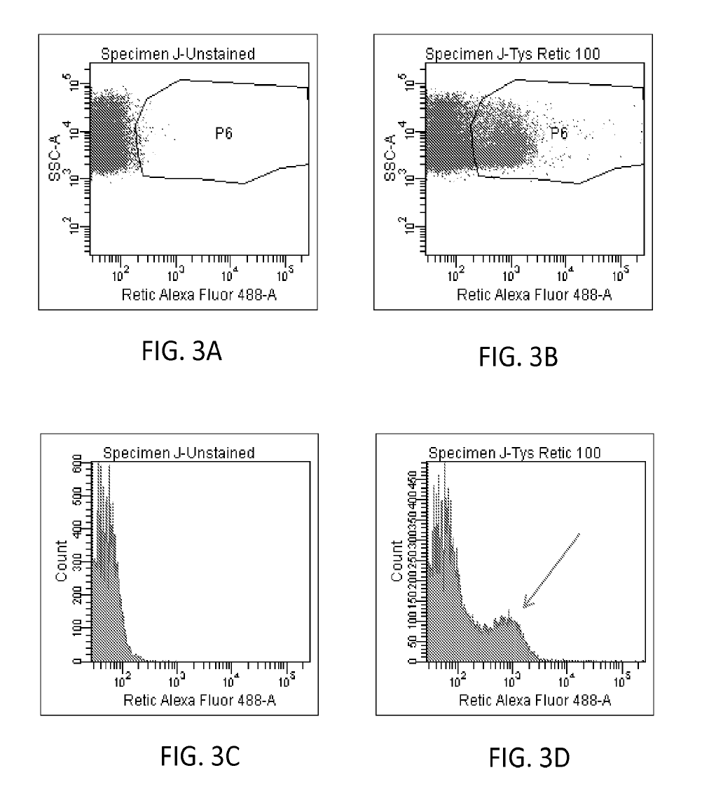

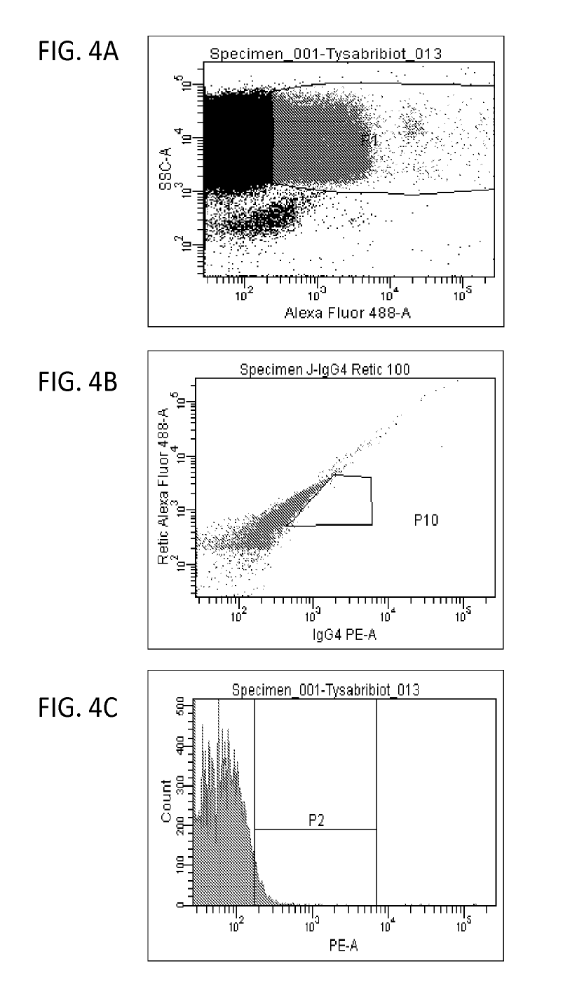

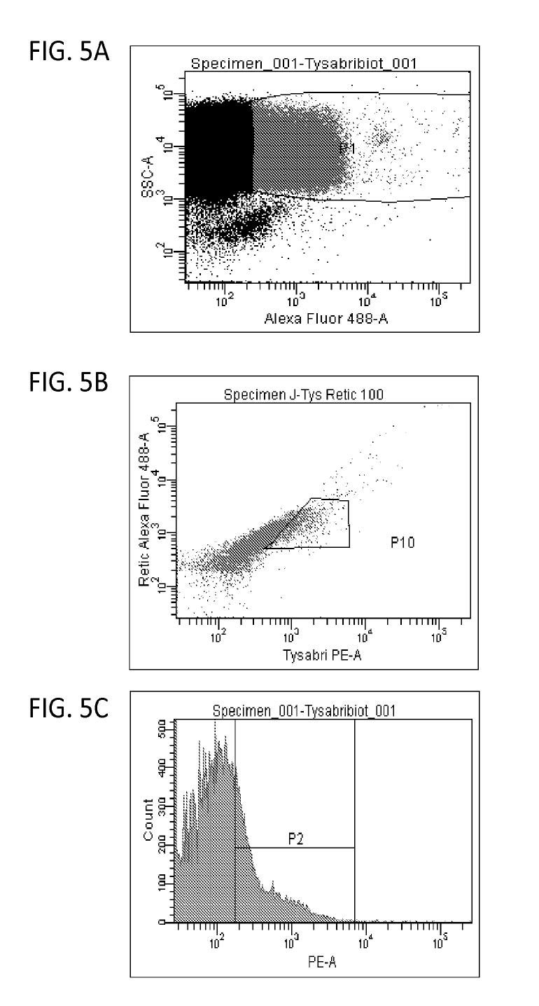

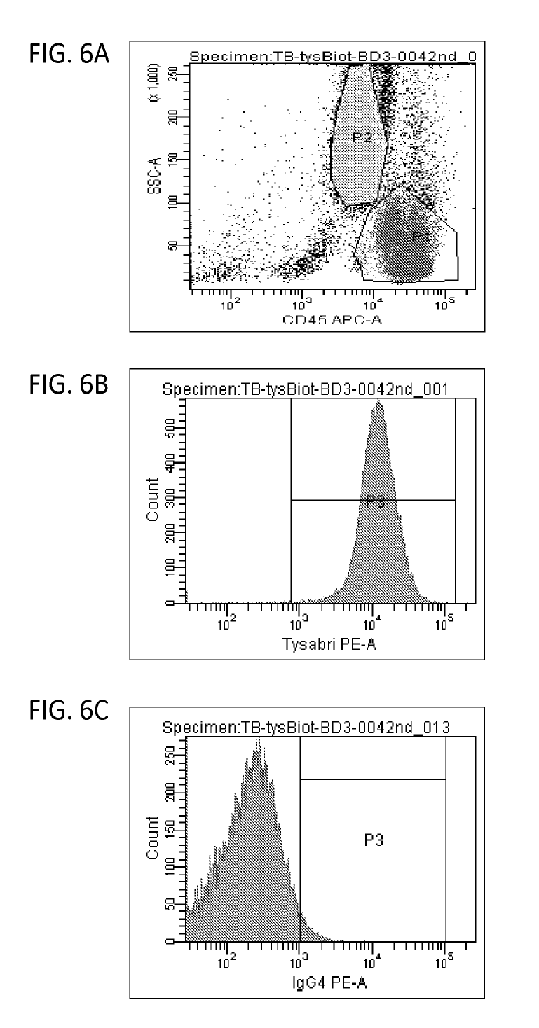

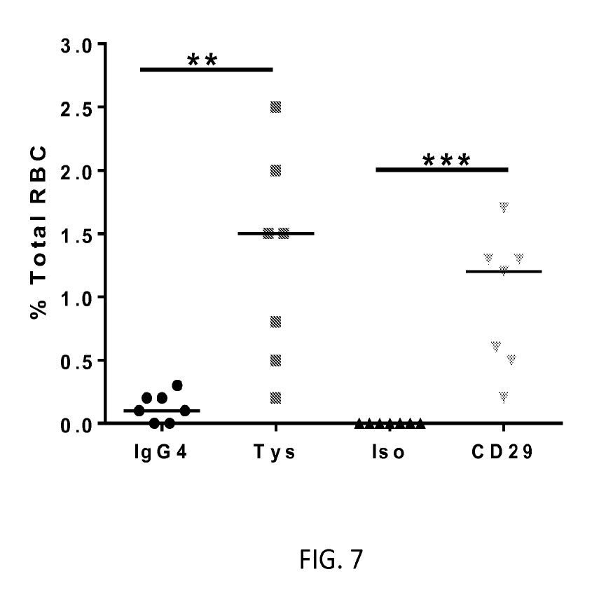

Reticulocytes in whole blood drawn from both sickle-cell disease and healthy subjects were first identified using thiazole orange staining. Cells that were double positive for thiazole orange and CD235 were gated as reticulocytes (FIG. 1A-1C). Reticulocyte content in peripheral blood based on thiazole orange staining ranged from 0.8 to 1.2% in healthy volunteers (FIG. 2A-2D). Reticulocyte percentage in sickle-cell patients showed a wide variation and ranged from 2 to 25% (FIG. 3A-3D). Both natalizumab and anti-CD29 antibody detected surface VLA-4 expression in reticulocytes gated using thiazole orange staining (FIG. 4A-4C, FIG. 5A-5C and FIG. 7). Reticulocytes from different donors showed a wide variation in the surface expression of VLA-4 revealed by staining, and correlated with the percentage of reticulocytes (FIG. 7). Conversely, VLA-4 staining was undetectable in reticulocytes from healthy controls with both the antibodies (data not shown). The results presented herein suggest that sickle-cell disease subjects have higher percentage of circulating reticulocytes which harbor VLA-4 integrin on the cell surface. Surface VLA-4 expression was detectable on mononuclear leukocytes from both sickle-cell and healthy subjects (FIG. 6A-6C).

### Natalizumab Showed Dose-Dependent Binding on Whole Blood Leukocytes and Reticulocytes in SCD.

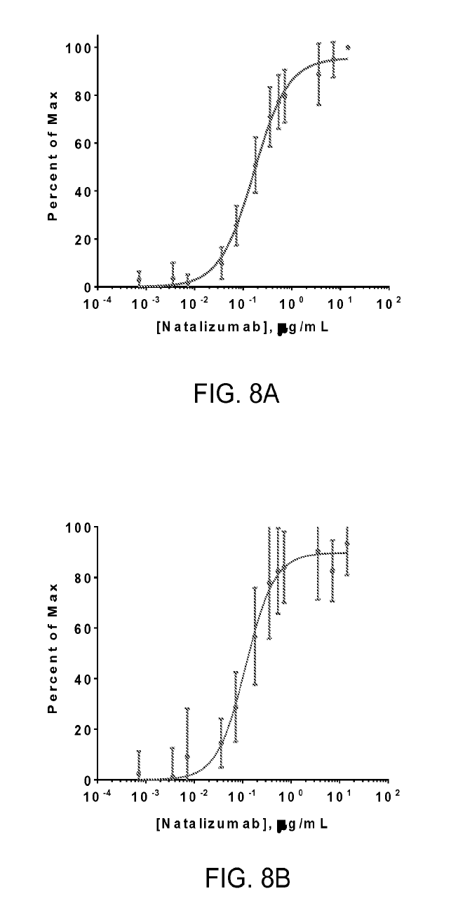

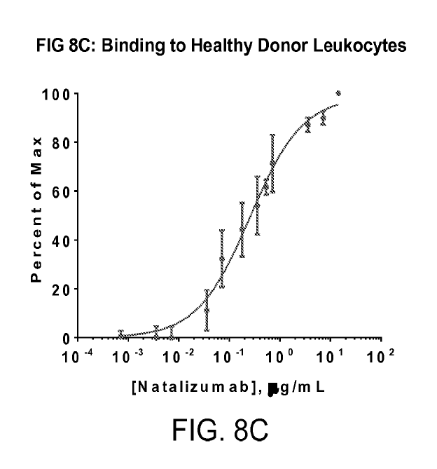

Next, it was determined if leukocytes and reticulocytes from sickle-cell blood samples have saturable levels of surface VLA-4. Minimally diluted whole blood from sickle-cell donors were stained with natalizumab at concentrations ranging from 0.001 to 20 μg/mL. VLA-4 surface expression was determined on cell subsets by gating CD45+ SSCLo mononuclear leukocytes and thiazole orange positive reticulocytes. Both leukocytes and reticulocytes exhibited a dose dependent saturation binding of natalizumab. One-site binding analyses illustrated that natalizumab saturation curves and EC50 values were similar in mononuclear leukocytes from healthy and sickle-cell disease subjects. Reticulocytes from SCD donors had a similar EC50 compared to mononuclear leukocytes, and approximately 10 fold less VLA-4 binding sites compared to mononuclear leukocytes from SCD donors. (FIG. 8A-8C).

### Natalizumab was Shown to Block SCD Reticulocyte and Leukocyte Adhesion Under Flow Conditions.

Adhesion of whole blood preparations to VCAM-1 were tested under physiologic flow conditions to determine the effect of natalizumab on total erythrocyte, reticulocyte, and WBC adhesion in the context of whole blood. In pediatric SCD blood samples, natalizumab (10 μg/mL) significantly inhibited whole blood adhesion to immobilized VCAM-1 during physiologic flow conditions when compared to baseline adhesion (61.8%+5.76; p=0.012). Increasing the natalizumab concentration (100 and 1000 μg/mL) did not further inhibit whole blood adhesion (55.4%+8.15 and 61.6%+7.62, respectively). In parallel, natalizumab significantly blocked adhesion of isolated leukocytes from sickle-cell donors when compared to baseline adhesion. Inhibition of WBCs to VCAM-1 was dose dependent with a maximum inhibition at 1 μg/mL natalizumab. Similar results were obtained from studies performed with blood samples from adult sickle-cell subjects (>18 years old). Lower doses of natalizumab were tested and block in adhesion was observed at 10, 1 and 0.1 μg/mL. Results were similar in whole blood as well as in isolated leukocytes and reticulocytes. The results presented herein collectively suggest that natalizumab can be effective in blocking cell adhesion in sickle-cell disease thereby reducing the occurrence of vaso-occlusive crisis.

IgG4 in circulation in humans undergoes chain exchange resulting in antibodies that are monovalent, i.e., having only one Fab arm specific for the antigen it targets. This phenomenon has been observed for nataluzimab, resulting in the conversion of divalent IgG4 to monovalent species. To investigate the impact of this conversion in saturation and adhesion assays, a monovalent natalizumab antibody was compared to the divalent form in both saturation and adhesion assays using isolated RBCs from sickle-cell subject samples. EC50 and IC50 calculated using one-site binding analyses suggested that both antibody species inhibited adhesion of SCD reticulocytes at 10 and 1 μg/mL concentration. However, at 0.1 μg/mL the divalent form appeared to be significantly more potent than the monovalent natalizumab in blocking reticulocyte adhesion to VCAM-1. Based on the IC50 from these studies, the results presented herein suggest that the monovalent antibody was 10 times less potent than divalent natalizumab in blocking reticulocyte adhesion.

### Discussion

Using an adhesion bioassay, the data provided herein are believed to provide the first report that natalizumab significantly decreased the adhesive interactions of whole blood components to VCAM-1 during physiologic flow conditions. Inhibition was observed in every patient sample although the individualized response to natalizumab as well as the cellular content of adherent cells varied from patient-to-patient. Natalizumab (10 μg/mL) decreased whole blood adhesion to VCAM-1 under physiologic flow conditions by an average of 62.8% (FIG. 7). This concentration is between 2 to 10-fold less than serum concentrations measured in multiple sclerosis patients treated with natalizumab. The results presented herein suggest that sickle cell patients may require similar or lower than current dosages of natalizumab to inhibit pathologic adhesive events. Natalizumab also significantly reduced the avidity of observed non-WBC adhesive interactions. The less avid nature of erythrocyte and reticulocyte adhesion at baseline may explain why natalizumab more effectively reduces adhesive avidity in erythrocytes and reticulocytes compared to WBCs. Similarly, natalizumab may demonstrate clinical effectiveness in the context of sickle cell disease at lower serum concentrations compared to concentrations required in multiple sclerosis, where the WBC adhesive interactions are thought to be the primary therapeutic target.

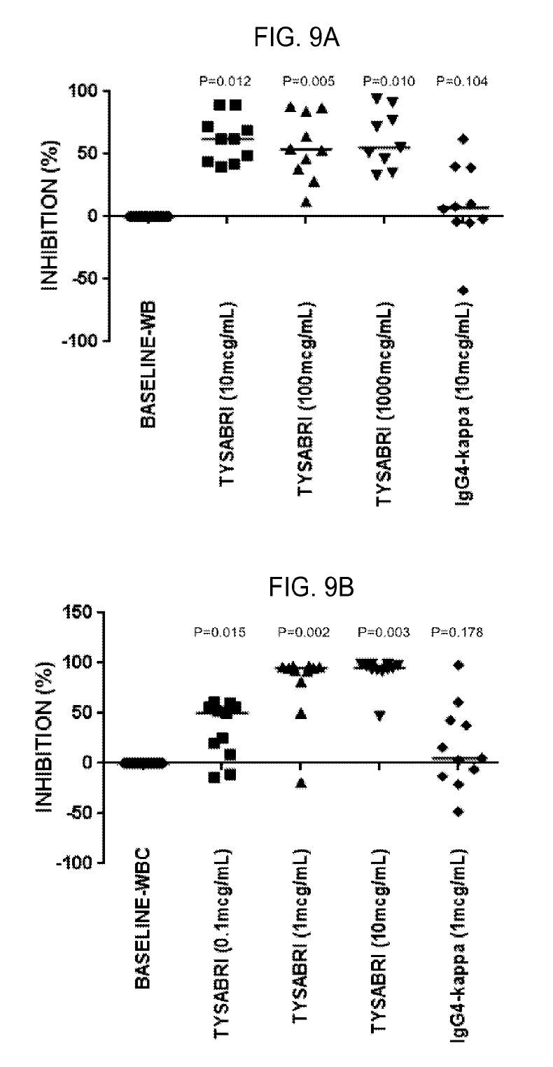

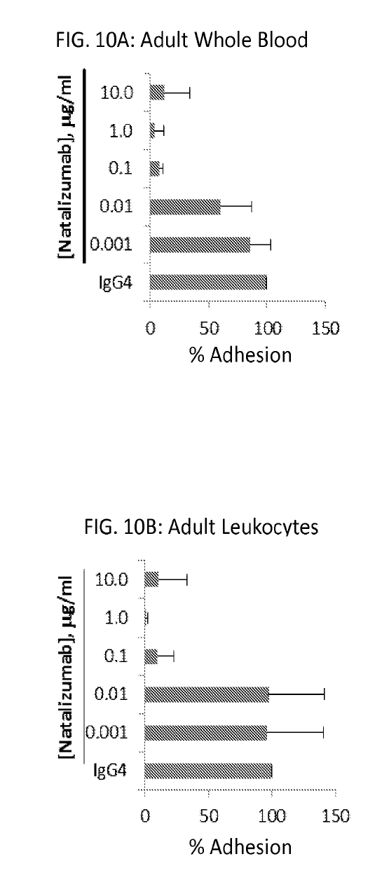

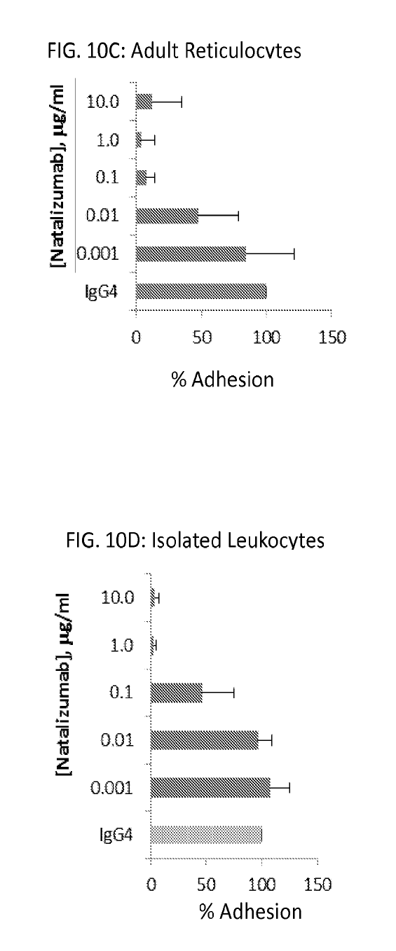

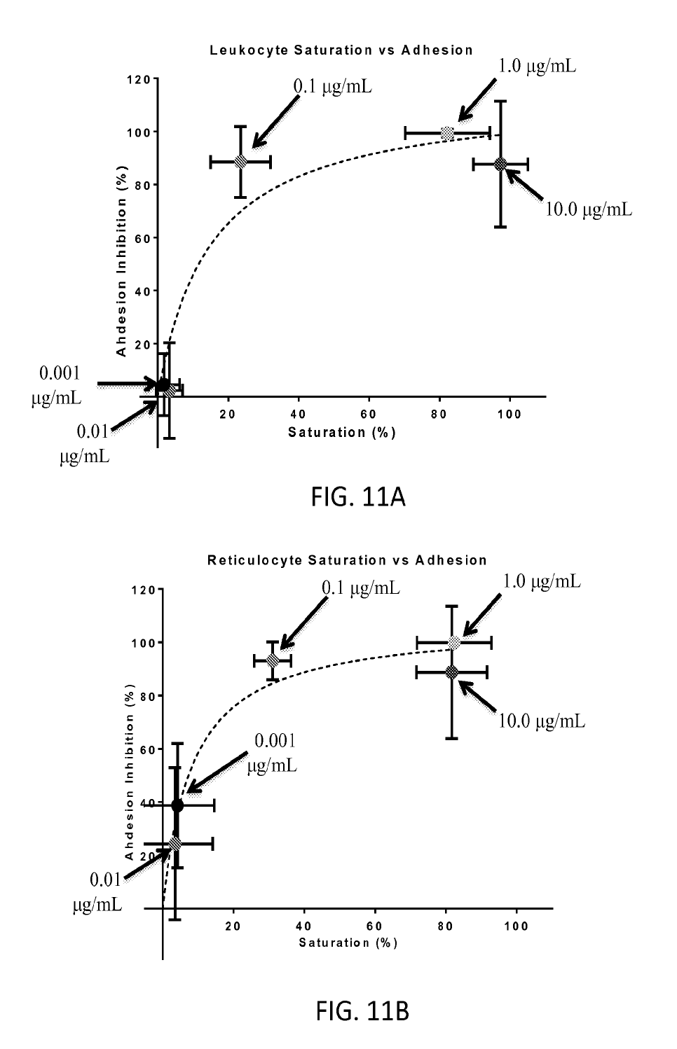

Baseline adhesion varied from patient to patient (65 to 4053 cells/mm2, median=293) in addition to the cell type supporting adhesion during each condition and the cell targeted during inhibition. Natalizumab was found to target reticulocytes in whole blood (average inhibition of 92%) and samples from patients with higher reticulocyte levels (>15%) tended to have greater inhibition. Therefore, the results presented herein suggest natalizumab may be most effective in patients with elevated reticulocyte counts. Natalizumab also inhibited adhesion of mature erythrocytes at 10 μg/mL, accounting for the majority of whole blood inhibition in patients 1 and 4 (FIG. 8B). Natalizumab inhibited adhesion of isolated WBCs, however WBC adhesion in the context of whole blood was minimal (FIG. 8B). The WBC adhesive interactions were much more avid compared to erythrocyte and reticulocyte adhesion (FIG. 9B). Thus, the results presented herein suggest elevated WBC levels may increase the opportunities for WBC-endothelial interactions. In the context of sickle cell disease, the data presented herein suggest that the potential clinical benefit of natalizumab may result more from preventing adhesive interactions of erythrocytes and reticulocytes compared to WBCs.

In patients where reticulocytes accounted for nearly 50% of adherent cells at baseline (patients 3, 6, and 7), inhibition of reticulocytes accounted for the majority of whole blood inhibition by natalizumab (FIG. 8B). Natalizumab also demonstrated significant inhibition of whole blood adhesion even when mature erythrocytes accounted for the majority of baseline adhesion (patients 1 and 4). The data presented herein suggest that the relatively mature erythrocytes in SCD express sufficient VLA-4 to support adhesion to VCAM-1 during physiologic flow conditions, and that natalizumab can target this population in addition to reticulocytes. The “mature” erythrocyte population in patients with sickle cell disease and other hemolytic anemias are relatively young (average age 30 days) compared to healthy controls (average age 90 days), thus natalizumab may have more clinical benefit in this population. Portions of the thiazole-orange staining reticulocytes in flow cytometry are CD71 negative, thus there may be reticulocytes represented in the CD71-/DAPI- population detected in flow adhesion experiments (data not shown).

The data presented herein are believed to provide the first evidence that natalizumab may be an effective anti-adhesive therapy for patients with SCD. In the flow adhesion bioassay presented herein, inhibition of adhesive interactions during flow conditions by pretreatment of whole blood suggests a role for natalizumab in the prevention of vasooclusive events. The microfluidic flow adhesion bioassay described herein may provide a platform to incorporate adhesive properties of whole blood into a clinical bioassay for preclinical anti-adhesive drug testing, and longitudinal assessment of patient response to anti-adhesive therapy. The effect of natalizumab on the prevention and reversal of vasooclusive events is further assessed in clinical studies.

### Example 2

### Natalizumab Inhibition of Adult SCD Blood Adhesion to VCAM-1

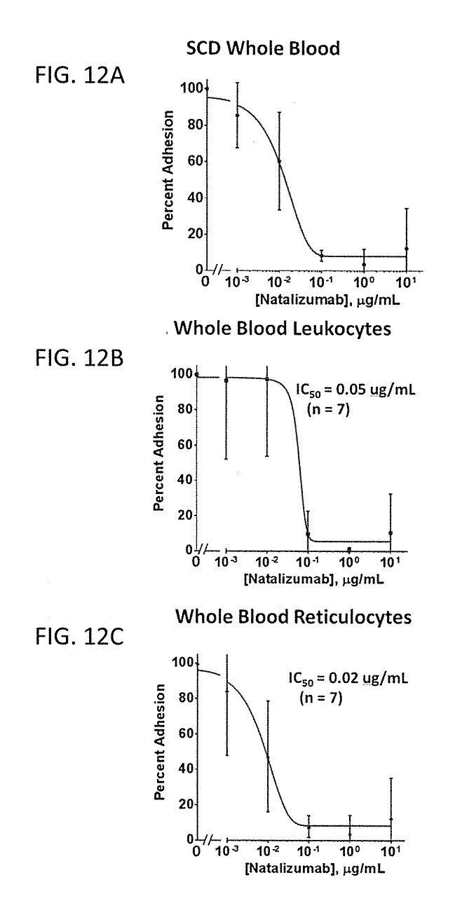

Whole blood samples from adult subjects with SCD were tested in the VCAM-1 adhesion assay under flow, and the inhibition of adhesion by natalizumab was evaluated. These studies included lower concentrations of natalizumab, 10 μg/mL and lower, well below trough levels observed in blood of patients with MS treated with 300 mg of natalizumab every 4 weeks (Rispens T et al. Anal Biochem 411(2):271-6 2011). Under these conditions, natalizumab inhibition of total whole blood cells was maximal and similar at 10, 1, and 0.1 82 g/mL. After fixing and staining with cell-specific markers, natalizumab was shown to block whole blood leukocytes and reticulocytes with a concentration of drug required for 50% inhibition (IC₅₀) of 0.05+0.03 μg/mL and 0.02+0.02 μg/mL, respectively (FIG. 12).

### Example 3

### Comparison of Monovalent and Divalent Forms of Natalizumab in Saturation and Adhesion Assays

Therapeutic IgG4 antibodies undergo chain shuffling while in circulation via a

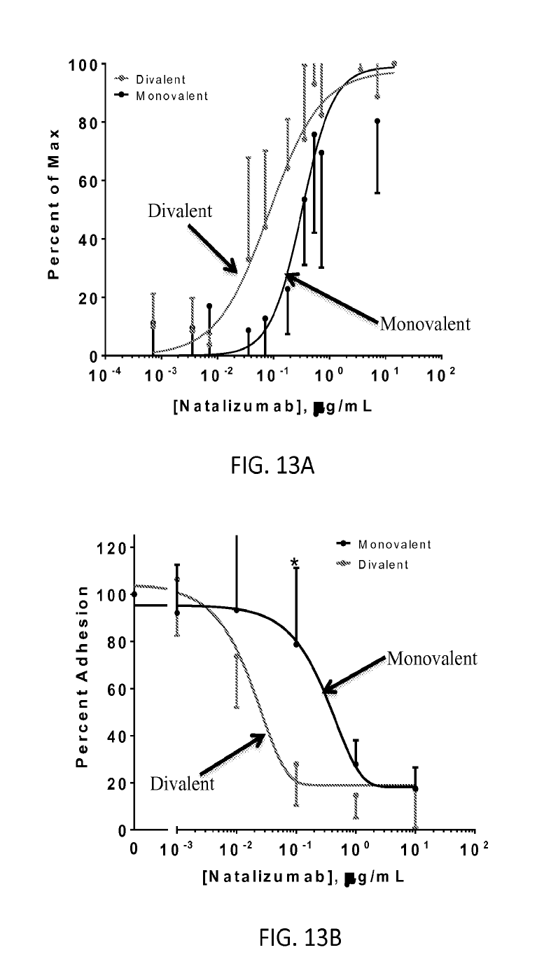

Fab arm exchange with endogenous IgG4 in humans, resulting in the production of antibodies that are potentially bispecific (Labrijn AF et al. Nat Biotechnol. 27(8):767-71 2009). This phenomenon has been reported for natalizumab as well, leading to the formation of a monovalent form of the antibody, with only one Fab arm specific for the α-subunit of VLA-4 (Rispens T et al. Anal Biochem. 411(2):271-6 2011; Shapiro RI et al. J Pharm Biomed Anal. 55(1):168-75 2011). To assess the impact of half-antibody exchange of natalizumab on saturation and adhesion of sickle cells, a synthetic monovalent form of natalizumab was used where 1 Fab arm was specific for VLA-4 and the other for CD4. To avoid interference by CD4+T-cells in VLA-4 saturation studies, isolated RBCs were used in assays comparing monovalent and divalent forms of natalizumab. The mean EC50 for divalent natalizumab on isolated RBCs from 5 SCD donors was 0.14+0.09 μg/mL (FIG. 13A) and similar to the EC50 of natalizumab on reticulocytes in SCD whole blood (FIG. 8). The EC50 for monovalent natalizumab was 0.89 +0.73 μg/mL or 7-fold higher compared to the divalent form, ranging from 1.7- to 12-fold for individual donors. Thus, the monovalent form binds with lower affinity compared to the bivalent parent antibody.

To understand the translation of lower binding affinity to inhibition of cell adhesion, the 2 forms were compared in the adhesion inhibition assay, where binding of isolated RBC to VCAM-1 was measured. At both 10 and 1 μg/mL, both the monovalent and divalent antibodies effectively blocked adhesion to VCAM-1 and were not significantly different. However, at 0.1 μg/mL, the inhibition of adhesion was significantly lower for the monovalent form compared to the divalent antibody (FIG. 13B). The IC₅₀ for inhibition of adhesion of reticulocytes in isolated RBC was 0.02+0.02 μg/mL, again similar to that for reticulocytes in SCD whole blood. The IC50 for the monovalent antibody was 0.37+0.33 μg/mL or 19-fold higher compared to the divalent form. Similar to the divalent form, monovalent natalizumab blocked cell adhesion at lower than saturating concentrations. The results presented herein suggest that, upon half-antibody exchange, there is a decrease in the extent of inhibition of adhesion of reticulocytes to VCAM-1. However, adhesion at 10 and 1 μg/mL was comparable, suggesting that, at trough concentrations, natalizumab behaves similarly regardless of whether it is monovalent or divalent.

### Example 4

### Rationale for Dosage and Duration in Human Treatment of SCD

Natalizumab doses were selected based on human data, PK/PD simulations, and nonclinical data. Natalizumab is commercially available as a 300 mg dose, given IV once monthly. This was based on a therapeutic dose equivalent to 4 mg/kg/month in patients with MS and CD. Additional human data are available for lower (approximately 150 mg) and higher (approximately 450 mg) IV monthly doses in subjects with MS and/or CD, including PK and PD data.

Three monthly natalizumab doses (administered every 28 days) were selected for safety and PK reasons. Based on data from MS and CD studies, as well as simulations, 3 monthly doses were determined to be sufficient to assess the effect of natalizumab on decreasing hemoglobin. Other safety measurements that are informed by 3 monthly doses are the incidence of anti-natalizumab antibody formation and hypersensitivity reactions. Based on MS and CD PK data, as well as simulations, 3 monthly doses are also considered sufficient to determine and/or estimate PK parameters in patients with SCD.

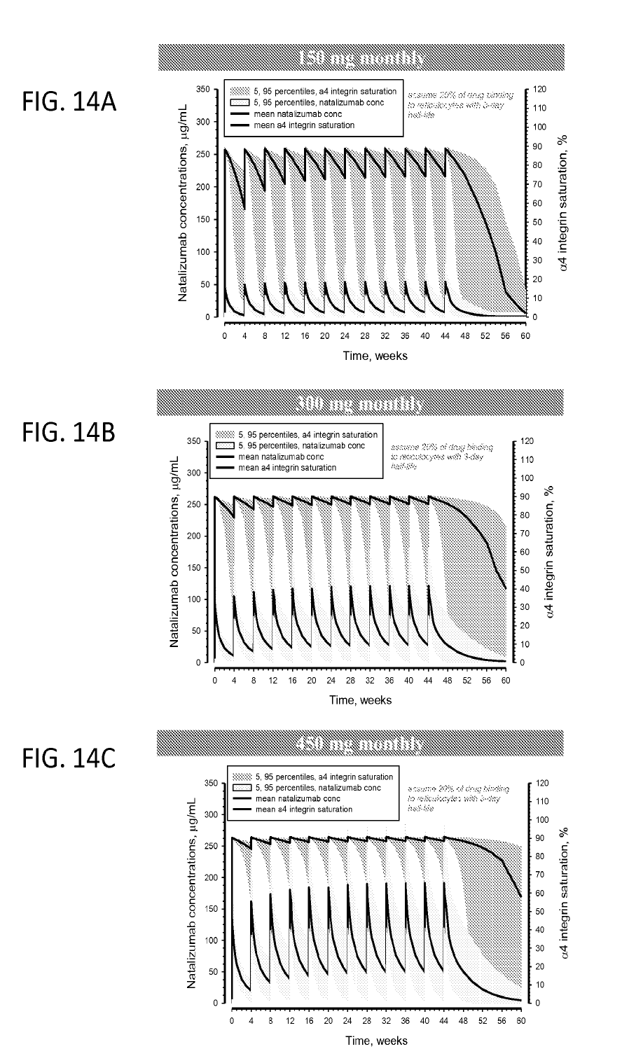

In prior natalizumab studies, natalizumab 300 mg IV monthly dosing resulted in >70% saturation of lymphocyte α4 integrin receptors. α4 integrin receptor saturation constitutes the primary PD endpoint measured in MS/CD natalizumab studies. In these studies, a direct relationship between natalizumab concentration (PK) and α4 integrin receptor saturation (PD) has been established. Given that the α4 integrin target is identical in MS and in SCD (i.e., VLA-4), this relationship was also used in SCD dose simulations as the primary determinant of target engagement (FIG. 14).

It is not known what level of reticulocyte/leukocyte α4 integrin receptor saturation is required to block reticulocyte/leukocyte adhesion in SCD. Nonclinical ex vivo adhesion data suggest that concentrations lower than that needed to achieve a clinically significant effect in MS/CD may be sufficient to block reticulocyte adhesion to VCAM-1 in SCD (See Example 1). Simulations of serum levels over time were run with the assumption that natalizumab bound to reticulocytes cannot return to circulation. Even under this assumption, the simulations showed that 150 mg IV monthly dosing achieved sufficient serum natalizumab levels to maintain a >20% α4 integrin receptor saturation. The commercially available 300 mg IV monthly dose was also selected due to its established safety profile and simulations, which suggest that this dose could achieve a >40% α4 integrin receptor saturation in the SCD population. A dose of 450 mg IV monthly was further selected, as simulations predicted that this dose could achieve a >70% α4 integrin receptor saturation in >90% of subjects, consistent with the known therapeutic threshold in MS and CD.

### Effect on Hemoglobin

Clinical experience has shown that circulating hemoglobin concentrations decrease after the initiation of natalizumab therapy. In patients with MS, who typically have hemoglobin in the normal range, hemoglobin concentrations are reduced on average by 5 to 10 g/L (0.5 to 1.0 g/dL), typically occurring within the first week following dose initiation and persisting through natalizumab therapy, but are still within normal limits in this non-anemic population. However, such decreases in hemoglobin may be clinically meaningful in patients with SCD who have significant anemia prior to natalizumab therapy. The mechanism of natalizumab-associated hemoglobin decreases is unclear, but given the lack of increase in total bilirubin, the known increase in erythroid precursors, and the lack of overt bleeding, it is thought to be via extravascular clearance (Robier C et al. Mult Scler. 2014).

Based on data from MS studies, a PK/PD model was developed to describe the relationship between natalizumab exposure and hemoglobin levels using an indirect link model. This model was used to predict the response to natalizumab in subjects with baseline (pre-natalizumab) hemoglobin levels between 70 to 90 g/L (7 to 9 g/dL), as well as 80 to 100 g/L (8 to 10 g/dL), which represent typical ranges of hemoglobin values in patients with SCD. Prophylactic doses of 150, 300, and 450 mg natalizumab given monthly were simulated.

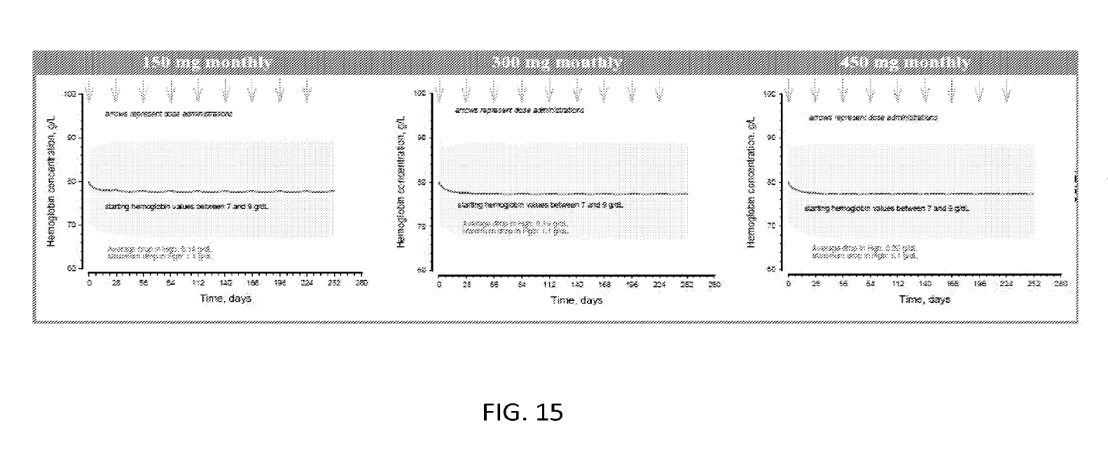

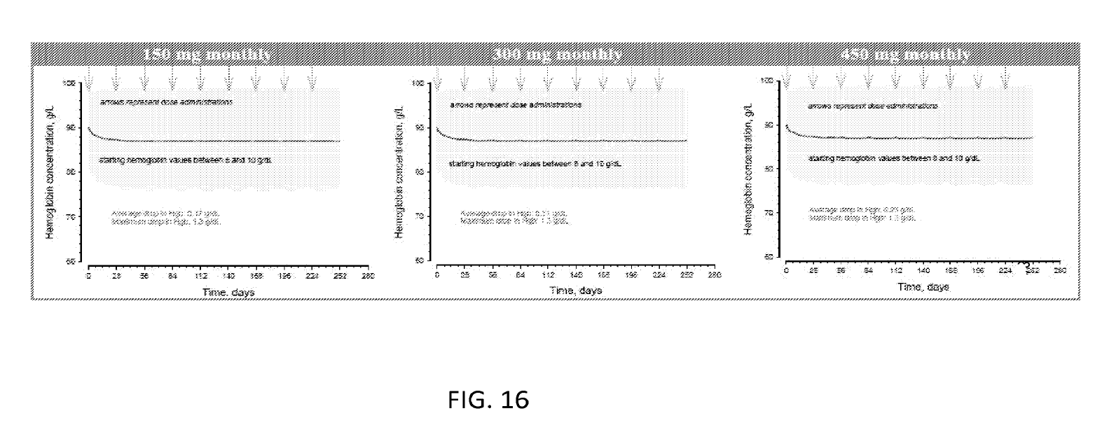

FIG. 15 and FIG. 16 show the simulated concentration-time profiles (mean and range) for hemoglobin at the various dose levels with initial hemoglobin concentrations of 70 to 90 g/L (7 to 9 g/dL) and 80 to 100 g/L (8 to 10 g/dL), respectively. In both cases, a drop in hemoglobin is predicted to reach steady-state approximately 2 weeks after dose initiation of natalizumab, as is the case with the observed data in the MS studies. The average drop is not expected to be large (1.4 to 2.3 g/L [0.14 to 0.23 g/dL], depending on the natalizumab dose and the initial hemoglobin range), with an anticipated maximum reduction of 11 to 13 g/L (1.1 to 1.3 g/dL, Table 2). Given the hypothesis that over time natalizumab blockade of α4 integrins will improve RBC transit time, decrease deoxygenation-associated hemoglobin polymerization, and thus improve RBC survival, it is expected that a counterbalancing effect will be increased hemoglobin over time. The simulations presented herein suggest that natalizumab will result in increased hemoglobin, particularly in hydroxyurea treated patients, to levels potentially >100 g/L (>10 g/dL), which have otherwise been suggested to increase blood viscosity and to be associated with adverse SCD events (Wun T et al. Hematology Reviews 1:e22 2009).

TABLE 2
Summary of Predicted Hemoglobin Decrease by Simulation

| | | Predicted | Predicted |
|---|---|---|---|
| Natalizumab | Hemoglobin | Average | Maximum |
| Dose | Range | Hb Decrease | Hb Decrease |
| (mg/month) | (g/dL) | (g/dL) | (g/dL) |
| 150 | 7-9 | 0.14 | 1.1 |
| 300 | 7-9 | 0.19 | 1.1 |
| 450 | 7-9 | 0.20 | 1.1 |
| 150 | 8-10 | 0.17 | 1.3 |
| 300 | 8-10 | 0.21 | 1.3 |
| 450 | 8-10 | 0.23 | 1.3 |

A conservative approach to limit enrollment to patients with entry hemoglobin ≧80 g/L (≧8 g/dL) (predicted nadir hemoglobin 77.0 to 78.6 g/L [7.70 to 7.86 g/dL], maximal decrease to 67 g/L [6.7 g/dL]) has been chosen with frequent safety monitoring of hemoglobin levels during the study. In addition, subjects will be permanently discontinued from study treatment if the subject has a decrease in hemoglobin to ≦55 g/L (≦5.5 g/dL) during the study or if the subject has a decrease in hemoglobin ≧25 g/L (≧2.5 g/dL) over a 7-day period. These hemoglobin safety thresholds were determined based on 1) decreases in hemoglobin greater than what is expected for SCD complications alone; 2) providing a safety margin for the detection of unanticipated red cell aplasia (typically defined as a rapid 30 g/L [3 g/dL] decrease in hemoglobin); and 3) remaining above a hemoglobin level of 50 g/L (5 g/dL), a level at which all patients would be expected to manifest symptomatic complications requiring evaluation and intervention (NHLBI NIH 02-2117 2002).

### Example 5

### Phase 1 Multiple-Ascending Dose Study

The Phase 1 study is a randomized, double-blinded, placebo-controlled, multiple-ascending dose study. The primary objective of the study is to evaluate the safety and tolerability of multiple-ascending IV doses of natalizumab administered monthly in subjects with SCD. Secondary objectives are to determine PK parameters, to evaluate α4 receptor saturation by natalizumab on reticulocytes and leukocytes, to evaluate levels of peripheral blood leukocytes, and to evaluate VCAM:Ig binding to reticulocytes and leukocytes. The study will be conducted in subjects with SCD because healthy volunteers are not thought to contain VLA-4 expressing reticulocytes and do not have a similar hemolytic process to evaluate the safety parameters related to anemia.

Three cohorts of 8 subjects will be enrolled. Subjects within each dose cohort will be continuously enrolled and randomized to receive 3 monthly IV natalizumab or placebo infusions (6:2 ratio) at the following proposed doses:
Cohort A: Natalizumab 150 mg or placebo
Cohort B: Natalizumab 300 mg or placebo
Cohort C: Natalizumab 450 mg or placebo

Dosing will be initiated with Cohort A. The Medical Monitor and Safety Surveillance Team (SST) will oversee the safety of subjects participating in this study.

The Medical Monitor will review each individual subject's safety data through the Day 15 Visit and the Day 43 Visit to make subsequent infusion decisions for each subject. The SST will review safety data collected through the Day 15 Visit from Cohorts A and B to make dose-escalation decisions. The SST will consist of at least a Safety and Benefit-Risk Management (SABR) physician, a biostatistician, and an independent hematologist, with ad hoc members added as required.

Natalizumab has been on the market for almost 8 years; globally, there are more than 300,000 person-years of experience with this product. However, there are special considerations that are specific to SCD, and thus, a Phase 1 study of natalizumab in patients with SCD is planned to:
Evaluate safety related to natalizumab-associated decreased hemoglobin.
Evaluate safety related to the consequence of natalizumab-induced mobilization of bone marrow-derived stem cell progenitors.
Evaluate safety related to increased numbers of circulating monocytes.
Evaluate PK and PD, given that reticulocytes are a cell target and response may be different in SCD than in MS or CD.
Evaluate the correlation between natalizumab dose and saturation of α4 integrins on SCD blood cells to inform subsequent clinical study designs.

Patients will be monitored for adverse events (AE), e g., immunogenicity, infusion and hypersensitivity reactions, mobilization of hematopoietic precursor cells, effects on hemoglobin, infections including Progressive Multifocal Leukoencephalopathy (PML) and hepatic injury.

The expected measurable therapeutic benefit of natalizumab in SCD will be a decrease in annualized VOC events. This is consistent with the proposed mechanism of action of natalizumab in SCD. Given the anticipated increase in RBC survival and improved vasculopathy hypothesized to occur with natalizumab in SCD, it is proposed to evaluate symptomatic improvement of patient-reported fatigue (as measured by an objective fatigue score) associated with an objective response (increased hemoglobin) as an alternative primary endpoint in natalizumab-treated patients. Other endpoints being considered include interval between VOC events (time to relapse, including 14- and 30-day hospital readmission rates), duration of VOC (annual inpatient hospital days, time to hospital discharge, and readiness for hospital discharge), annual VOC rate by concomitant hydroxyurea use, pain (patient-reported pain score and outpatient opiate use), change in baseline hemoglobin, change in baseline reticulocyte count, change in lactate dehydrogenase, JCV seropositive prevalence, and development of anti-natalizumab antibodies.

### Example 6

### Exemplary Phase 1 Multiple-Ascending Dose Study of the Safety, Tolerability, and Pharmacokinetics of Intravenous Natalizumab in Subjects with Sickle Cell Disease

### Product Name: Natalizumab

IND Number: Not applicable

### Protocol Title: A Phase 1, Randomized, Double-Blinded, Placebo-Controlled, Multiple-Ascending Dose Study of the Safety, Tolerability, and Pharmacokinetics of Intravenous Natalizumab in Subjects With Sickle Cell Disease

### Study Phase: 1

Indication: Sickle cell disease
Background and Rationale for Study: Natalizumab is a recombinant humanized immunoglobulin (Ig) G4κ monoclonal antibody that binds to α4 integrins on reticulocytes and leukocytes, inhibiting the ability of these cells to adhere to the vascular endothelium. Natalizumab has been approved in the United States (US), the European Union (EU), and other countries around the world for the treatment of patients with multiple sclerosis (MS) and in the US for the treatment of patients with moderate to severe Crohn's Disease (CD). In MS and CD, natalizumab's effect is based on inhibiting leukocyte adhesion and their transmigration into tissues. Natalizumab is now being developed for the treatment of sickle cell disease (SCD). It is hypothesized that this is based on its ability to block reticulocyte and leukocyte adhesion.

Natalizumab binds to the α4 subunit of α4β1 (Very Late Antigen-4, VLA-4) on the surface of reticulocytes and leukocytes. In SCD, it is believed that VLA-4 mediates the adhesion of reticulocytes and leukocytes to the vascular endothelium via binding to vascular cell adhesion molecule-1 (VCAM-1) on the endothelial surface. Both the number of circulating VLA-4 positive reticulocytes and the abundance of endothelial VCAM-1 are markedly increased in individuals with SCD compared to individuals without SCD (see Example 1). VLA-4 on SCD blood cells can also mediate cell-to-cell adhesion either directly or indirectly through bridging molecules such as fibronectin. VLA-4-mediated adhesive interactions have been implicated in SCD pathophysiology leading to vaso-occlusive events (VOC) and hemolytic anemia, the clinical hallmarks of SCD. By blocking VLA-4, natalizumab has the potential to decrease adverse adhesive interactions in SCD patients, leading to reduced disease severity, including prevention of VOC and decreasing hemolysis.

It has been established that as natalizumab concentration increases, α4 integrin receptors on leukocytes become saturated, and the cells are inhibited from binding to endothelium and entering tissues. As a consequence, leukocyte numbers are increased in circulation (leukocytosis). Further, as leukocyte α4 integrin receptors (i.e., VLA-4) are blocked by natalizumab, the ability of these cells to bind VCAM-1 decreases proportionately. This phase 1 study will determine the pharmacokinetic (PK) and pharmacodynamic (PD) for reticulocytes in patients with SCD.

This Phase 1 study is designed to assess the PK, safety, and tolerability of natalizumab in patients with SCD, with specific emphasis on the effect on pre-existing anemia. The study will also provide information regarding the relationship between PK and PD biomarkers, which will form the basis for dose selection for future efficacy studies.

The study is a placebo-controlled, multiple-ascending dose design investigating up to three monthly natalizumab or placebo infusions at up to three dose levels: 150, 300, and 450 mg. Individual subject safety data will be evaluated prior to repeat infusion, and the safety data from the first infusion within each dose cohort will be evaluated prior to escalating to the next dose level.

### Study Objectives and Endpoints:

### Objectives

Primary: The primary objective of the study is as follows:

To evaluate the safety and tolerability of multiple-ascending intravenous (W) doses of natalizumab administered monthly in subjects with SCD

Secondary: Secondary objectives of this study are as follows:

To determine PK parameters of multiple-ascending doses of IV natalizumab in subjects with SCD

To evaluate α4 receptor saturation by natalizumab on reticulocytes and leukocytes following multiple-ascending doses of IV natalizumab in subjects with SCD

To evaluate levels of peripheral blood leukocytes following multiple-ascending doses of IV natalizumab in subjects with SCD

To evaluate VCAM:Ig binding to reticulocytes and leukocytes following multiple ascending doses of IV natalizumab in subjects with SCD

Exploratory objective of this study is as follows:

To assess the effect of IV natalizumab on PD markers in subjects with SCD, which includes hematologic, inflammatory, coagulation, and adhesion biomarkers

To assess the effect of IV natalizumab on PD markers in subjects with SCD using exploratory research assays, which may include, but are not limited to, assays for cell adhesion under flow shear conditions and cell-to-cell aggregates

To assess the impact of reticulocyte count on the PK and PD parameters (i.e., α4 receptor saturation and VCAM:Ig binding on reticulocytes and leukocytes)

### Endpoints

### Primary:

Incidence of adverse events (AEs) and serious adverse events (SAEs), including symptomatic anemia, VOC, hypersensitivity reactions, and clinically significant laboratory abnormalities

Incidence of serum anti-natalizumab and neutralizing antibody formation

### Secondary:

Serum natalizumab PK parameters, including area under the curve of the plasma drug concentrations to infinity (AUC_{inf}), peak plasma drug concentration (Cₘₐₓ), time to peak drug concentration (Tₘₐₓ), and terminal half-life (t_{1/2})

Percentage of α4 receptor saturation by natalizumab on reticulocytes and leukocytes

Intensity of α4 receptor staining (level of α4 expression determined by flow cytometry) on reticulocytes and leukocytes

Level of peripheral blood leukocytes

Level of soluble VCAM:Ig adhesion to reticulocytes and leukocytes

### Exploratory:

Changes in hematologic parameters, inflammatory markers, coagulation markers, and adhesion biomarkers associated with SCD clinical severity

Change in ex vivo adhesion of subject's blood cells under flow shear conditions

Level of peripheral blood reticulocytes

Study Design: This is a Phase 1, randomized, double-blinded, placebo-controlled, multiple-ascending dose study to assess the safety, tolerability, and PK of IV natalizumab in subjects with SCD.
Rationale for Dose and Schedule Selection: Natalizumab doses were selected based on available human data, PK/PD simulations, and nonclinical data. Natalizumab is commercially available as a 300 mg dose, given IV once monthly. This was based on a therapeutic dose equivalent to 4 mg/kg/month in patients with MS and CD. Additional human data are available for lower (approximately 150 mg) and higher (approximately 450 mg) IV monthly doses in subjects with MS and/or CD, including PK and PD data.

In prior natalizumab studies, natalizumab 300 mg IV monthly dosing resulted in >70% saturation of leukocyte α4 integrin receptors. α4 integrin receptor saturation constitutes the primary PD endpoint measured in MS/CD natalizumab studies, with a direct relationship between natalizumab concentration (PK) and α4 integrin receptor saturation (PD). Given that the α4 integrin target is identical in MS and in SCD (i.e., VLA-4), this relationship was also used in SCD dose simulations as the primary determinant of target engagement.

It is not known what level of reticulocyte/leukocyte α4 integrin receptor saturation is required to block reticulocyte/leukocyte adhesion in SCD. Nonclinical ex vivo adhesion data suggest that concentrations lower than that needed to achieve a clinically significant effect in MS/CD may be sufficient to block reticulocyte adhesion to VCAM-1 in SCD (see Example 1). Simulations showed that 150 mg IV monthly dosing achieved sufficient serum natalizumab levels to maintain a >20% α4 integrin receptor saturation. The commercially available 300 mg IV monthly dose was also selected due to its established safety profile and simulations, which suggest that this dose could achieve a >40% α4 integrin receptor saturation in the SCD population. A dose of 450 mg IV monthly was further selected, as simulations predicted that this dose could achieve a >70% α4 integrin receptor saturation in >90% of subjects, consistent with the known therapeutic threshold in MS and CD.

Duration of Study Participation: The overall duration of the study for each subject, following a <28-day Screening Period, will be approximately 6 months: a 3-month Treatment Period (IV treatment at Day 1 and every 4 weeks for up to 3 administrations), and a 3-month Follow-up Period after the last infusion.

Study Location: Approximately 10 sites in the US
Number of Planned Subjects: Approximately 24 subjects (6:2 ratio of natalizumab:placebo per dose cohort) will be enrolled in this study. Subjects who withdraw prior to completion of study follow-up may be replaced. In addition, assuming there are no identified safety issues, based upon the recommendation and approval of the Safety Surveillance Team (SST), up to 3 additional cohorts to further assess the safety, PK, and/or PD in “expansion cohorts” at one or more dose levels may be added. Thus, a maximum of 48 subjects could be enrolled.
Sample Size Determination: The sample size is not based on statistical considerations. Eight subjects per cohort (6:2 ratio of natalizumab:placebo) are thought to be sufficient to characterize the safety, tolerability, and the PK profile.

### Study Population:

### Inclusion Criteria

To be eligible to participate in this study, candidates must meet the following eligibility criteria at Screening or at the timepoint specified in the individual eligibility criterion listed:

1. Ability to understand the purpose and risks of the study and provide signed and dated informed consent and authorization to use protected health information (PHI) in accordance with national and local subject privacy regulations.

2. Aged 18 to <70 years old, inclusive, at the time of informed consent.

3. Subjects of childbearing potential must practice effective contraception during the study and be willing and able to continue contraception for 3 months after their last dose of study treatment.

4. Must have a diagnosis of SCD (homozygous disease [HbSS] or heterozygous disease with beta 0-thalassemia [HbS-α° -thalassemia]) confirmed by hemoglobin analysis.

5. If receiving hydroxyurea, treatment must have been prescribed for at least

6 months with the dose stable for at least 3 months and with an absolute neutrophil count (ANC) >2500 cells/μL at Screening and prior to each dose (i.e., Days 1, 29, and 57).

6. Must have hemoglobin >8 g/dL at Screening and prior to the first dose (i.e., Day 1). Subjects with hemoglobin <8 g/dL but >7.5 g/dL during Screening may be allowed to participate and continue in the study provided that repeat hemoglobin determination on Day −10±4 days is >8 g/dL.

7. Must be willing to remain free from initiating therapy with any immunosuppressive or immunomodulatory treatment for the duration of the study.

8. Must be in stable clinical condition at the time of first dose, as determined by the Investigator.

9. Must have reliable IV access, as determined by the Investigator.

### Exclusion Criteria

Candidates will be excluded from study entry if any of the following exclusion criteria exist at Screening or at the timepoint specified in the individual criterion listed:

10. History of or positive test result at Screening for human immunodeficiency virus (HIV).

11. History of or positive test result at Screening for hepatitis C virus (HCV) antibody or current hepatitis B infection (defined as positive for hepatitis B surface antigen [HBsAg] and/or hepatitis B core antibody [HBcAb]). Subjects with immunity to hepatitis B from either active vaccination (defined as negative HBsAg, positive hepatitis B surface antibody [HBsAb], and negative HBcAb) or from previous natural infection (defined as negative HBsAg, positive HBsAb IgG, and positive HBcAb) are eligible to participate in the study (definitions are based on the US Centers for Disease Control (CDC) and Prevention's interpretation of the hepatitis B serology panel).

12. Any prior use of natalizumab.

13. A diagnosis of hereditary persistence of fetal hemoglobin (HPFH) or δ-β thalassemia.

14. Contraindications to or inability to undergo magnetic resonance imaging (MRI), e.g., any type of electronic, mechanical, or magnetic implant (cardiac pacemaker, aneurysm clips, implanted cardiac defibrillator), potential ferromagnetic foreign body (metal slivers, metal shavings, other metal objects), claustrophobia that cannot be medically managed, inability to lie still, or body weight/girth exceeding the limitations of the MRI machine aperture.

15. Major surgery <8 weeks prior to Screening or scheduled surgery during the Treatment Period.

16. Blood transfusion within 30 days of first dose or on chronic transfusion therapy.

17. Prior hematopoietic stem cell transplantation (HSCT).

18. History of any clinically significant cardiac, endocrine (such as diabetes), hepatic, immunologic, metabolic, urologic, pulmonary, neurologic (except history of stroke with no or stable neurologic deficits for >6 months), dermatologic, psychiatric, muscular, ophthalmic, and renal conditions, or other major disease (with the exception of SCD), as determined by the Investigator. 19. Abnormal liver function test results at Screening: alanine aminotransferase (ALT) or aspartate aminotransferase (AST) >3× the upper limit of normal (ULN).

20. Estimated glomerular filtration rate (eGFR) <45 mL/min/1.73 m² at Screening.

21. Twelve-lead electrocardiogram (12-lead ECG) result(s) considered to be clinically significant by the Investigator.

22. History of progressive multifocal leukoencephalopathy (PML) or other opportunistic infections prior to Screening.

23. History of malignant disease, including solid tumors and hematologic malignancies (with the exception of basal cell and squamous cell carcinomas of the skin that have been completely excised and are considered cured).

24. Serious infection (e.g., cellulitis, abscess, pneumonia, septicemia) within 30 days prior to Screening.

25. Active bacterial or viral infection.

26. Vaccinations within 8 weeks of first dose or live, attenuated vaccination planned during study duration.

27. Exposure to IV immunoglobulin, monoclonal antibodies, cytokines, growth factors (including hematopoietic growth factor therapy), soluble receptors, other recombinant products, or fusion proteins within 3 months prior to Screening.

28. Patients on dialysis.

29. Treatment with IV and/or oral corticosteroids (topical or inhaled corticosteroids are acceptable) or related products within 30 days prior to Screening.

30. Current chemotherapy or use of immunosuppressant medications (e.g., mitoxantrone, cyclophosphamide, cyclosporine, azathioprine, methotrexate, mycophenolate, rituximab) with the exception of hydroxyurea for SCD within 12 months prior to Screening.

31. The subject is considered by the Investigator to be immunocompromised.

32. Prior treatment with any of the following: total lymphoid irradiation, anti-T cell therapy, or T-cell or T-cell receptor vaccination.

33. Inability to comply with study requirements.

34. Other unspecified reasons that, in the opinion of the Investigator or Sponsor, make the subject unsuitable for enrollment.

35. Female subjects who are pregnant (as confirmed by a positive pregnancy test result at Screening) or currently breastfeeding.

36. History of drug or alcohol abuse (as defined by the Investigator) <6 months prior to Screening.

37. Current enrollment in any other drug, biologic, or clinical study, or treatment with an investigational drug <30 days (or 5 half-lives of the agent, whichever is longer) prior to Screening.

### Treatment Groups:

Three cohorts of 8 subjects will be enrolled. Subjects within each dose cohort will be continuously enrolled and randomized to receive three monthly IV natalizumab or placebo infusions (6:2 ratio) at the following proposed doses:

Cohort A: Natalizumab 150 mg or placebo
Cohort B: Natalizumab 300 mg or placebo
Cohort C: Natalizumab 450 mg or placebo

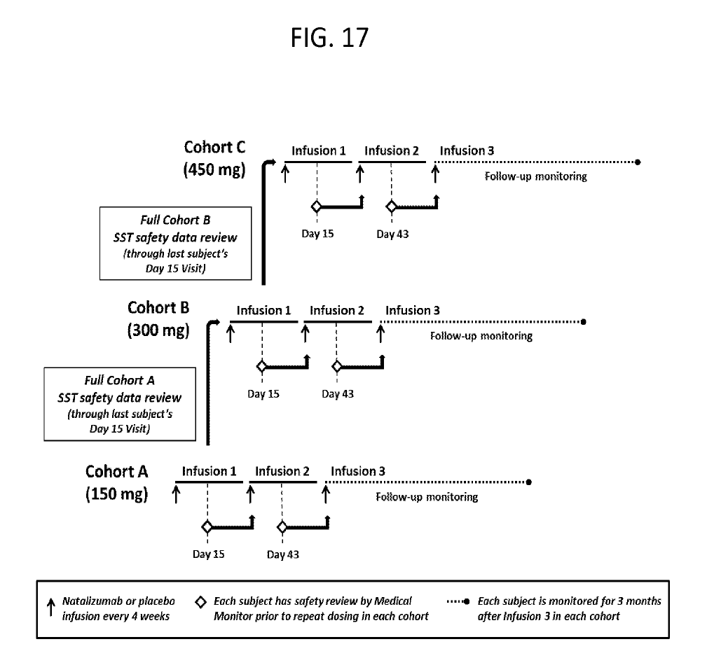

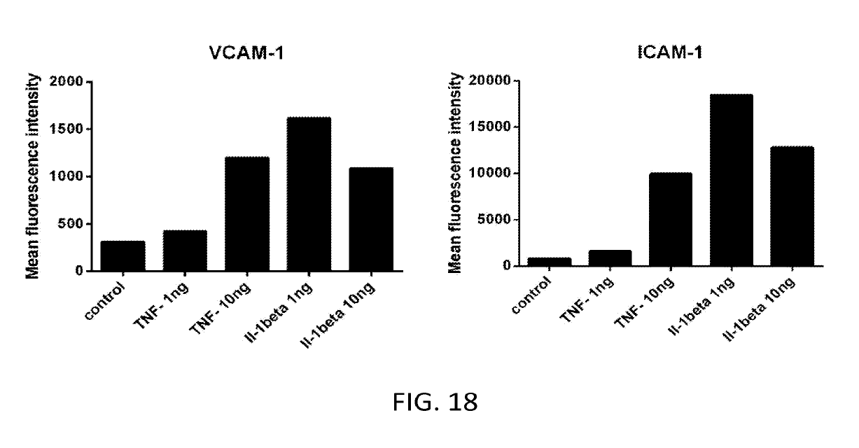

The dosing scheme for each cohort and dose escalation between cohorts is illustrated in FIG. 17. Dosing will be initiated with Cohort A. Following each infusion of placebo or natalizumab, each individual subject's safety data through the Day 15 Visit and the Day 43 Visit will undergo blinded review by a Medical Monitor prior to the subject advancing to the next infusion. If more than one subject in a cohort must discontinue treatment for safety reasons, the SST will perform an unblinded review of all available data to determine if the study should be terminated. The SST will consist of at least a Safety and Benefit-Risk Management (SABR) physician, a biostatistician, and an independent physician (i.e., independent hematologist), with ad hoc members added as required. If deemed safe to continue with subsequent infusions, then the Medical Monitor will resume blinded review of each individual subject's safety data through the Day 15 Visit and the Day 43 Visit.

Once the last subject infused in a cohort reaches Day 15, all available data will undergo unblinded safety review by the SST prior to initiating the next cohort. Data from a minimum of 6 subjects in the cohort must be available for SST review. If more than 2 subjects in the cohort under review have been withdrawn from the study prior to Day 15 for reasons unrelated to safety, the subjects will be replaced to achieve a sufficient data set for review.

Visit Schedule: Subjects will have 19 visits over a 6-month period during the study.

Screening Visit (one or more visits) within 28 days before infusion on Day 1.

Inpatient period for Infusions 1 and 3 lasting from Day 1 to Day 2 (24 hours after Infusion 1) and from Day 57 to Day 58 (24 hours after Infusion 3), respectively.

Outpatient Infusion Center visit for Infusion 2 on Day 29.

Outpatient safety, PK, and PD visits on Days 4, 8, 15, and 22 following Infusion 1; on Days 36, 43, and 50 following Infusion 2; on Days 60, 64, 71, 78, 85, 117, and 147 during the Follow-up Period.

Discontinuation of Treatment: Unless otherwise indicated, a subject must permanently discontinue study treatment for any of the following safety reasons:
A life-threatening SAE, considered to be related to natalizumab by the Investigator and/or Sponsor

The subject experiences a systemic hypersensitivity reaction following study treatment administration. Systemic hypersensitivity reactions are immediate-type reactions (typically occurring within 2 hours of the start of IV infusion) that are usually associated with angioedema and urticaria (e.g., anaphylaxis).

The subject has hemoglobin <5.5 g/dL during the study.

The subject develops an acute hemoglobin decrease of >2.5 g/dL over <7 days not explained by the subject's underlying SCD, as determined by the Investigator.

The subject has a diagnosis of confirmed PML or is suspected of having PML or other significant opportunistic infection.

Unless otherwise indicated, a subject must permanently discontinue study treatment for any of the following additional reasons:

The subject becomes pregnant.

Treatment delays of >21 days for any reason.

The subject starts taking hydroxyurea or increases their hydroxyurea dose.

The subject withdraws consent.

At the discretion of the Investigator or SST for medical reasons.

At the discretion of the Investigator, Sponsor, or SST for noncompliance.

Efficacy Assessments: No efficacy measures will be assessed in this Phase 1 study.

### Safety Assessments:

Monitoring of AEs and SAEs

Physical examinations (including height and weight measurements and 12-lead ECG at Screening)

Neurological evaluation, including identification of signs and symptoms suggestive of PML, neurologic review of systems, and targeted neurologic exam (mental status, cranial nerve, reflex, visual, motor/cerebellar, and sensory evaluations)

Vital sign measurements: supine systolic and diastolic blood pressure, heart rate, oxygen saturation, respiratory rate, and body temperature

Hematologic monitoring (complete blood count with differentials including absolute reticulocyte count and ANC)

Serum chemistry, including liver and renal panels and lactate dehydrogenase (LDH)

LDH isoenzymes

Urinalysis

Anti-natalizumab and neutralizing antibodies

Anti-JCV antibodies

Blood cell immunophenotyping (CD34+)

Pregnancy testing

Preinfusion checklist completion

Monitoring of concomitant therapy

### PK Assessments:

Serum natalizumab concentration, including AUC_{inf}, Cₘₐₓ, Tₘₐₓ, and t_{1/2}

### PD Parameters:

α4 integrin saturation on reticulocytes and leukocytes

SUBSTITUTE SHEET (RULE 26)

α4 expression level on reticulocytes and leukocytes

Leukocyte and differential leukocyte cell count

VCAM:Ig flow binding

### Exploratory PD Parameters:

Hematologic markers (hemoglobin, urine hemoglobin, LDH, reticulocyte count)

Markers of coagulation activation (F1.2, D-dimer, thrombin-antithrombin III complex [TAT])

Inflammation markers (high-sensitivity C-reactive protein [hsCRP])

Adhesion markers (e.g., soluble VCAM)

Research assays, which may include, but are not limited to, assays for cell adhesion under flow shear conditions and cell-to-cell aggregates

DNA/RNA/Proteomic Sample Collection (optional): Yes
Rationale: Samples, including remaining aliquots from other analyses, will be archived for up to 15 years after the end of the study and may be studied to characterize potential biomarkers (e.g., DNA, RNA, and proteomic analysis) associated with the effects of natalizumab treatment, including immune function, SCD disease, and possible risk factors related to JCV and development of PML.
Statistical Statement and Analytical Plan: Safety data will be summarized by dose level and compared to placebo. Subjects assigned to placebo for all cohorts will be treated as a single group. Descriptive statistics will be used to summarize PK, PD, and exploratory parameters by dose level and compared to placebo.
Interim Analysis: No formal interim analysis will be performed. Safety data will be reviewed on an ongoing basis for the purpose of safety monitoring and dose escalation decisions.
Ongoing Data Monitoring Plan (ODMP): A formal ODMP will not be created for this study. The Medical Monitor will review data from each subject for subsequent infusion determinations, and the SST will review data from each cohort for dose escalation decisions. Additionally, the SST will review data from all subjects in the event of an individual subject dose termination for continued dosing determination in any subject.
Study Stopping Rules: The Medical Monitor and SST will oversee the safety of subjects participating in this study. The Medical Monitor will review each individual subject's safety data through the Day 15 Visit and the Day 43 Visit to make subsequent infusion decisions for each subject. The SST will review safety data collected through the Day 15 Visit from Cohorts A and B to make dose escalation decisions. Before escalating to the next higher dose, there will be agreement after unblinded review that the current emerging safety and tolerability data support dose escalation. If 2 or more subjects in a cohort achieve a dose termination decision for safety reasons, the SST will review all available safety data prior to continued dosing of any subject enrolled at that point.

The SST will also meet on an ad hoc basis, if required, to address any safety issues of concern. At any time during the study, the SST may recommend continuation of the study without modification, discontinuation of further enrollment into a treatment group, or discontinuation of further enrollment for the entire study.

### Dosing Suspension

If 1 SAE occurs in a subject that is considered at least possibly related to study treatment by the Investigator, further dosing will be suspended until full evaluation by the SST. If the event occurred in a subject treated with natalizumab, depending on its nature, severity, and outcome, the SST will make a decision to either request additional safety data, stop the study, continue dosing additional subjects within specified cohort(s) only, or continue dosing all subjects in all cohorts. Dosing will not resume a safety evaluation has been completed and the Investigator has received written approval to resume dosing.

If a subject experiences a VOC, then dosing will be suspended until 1 week after resolution of the VOC, or until deemed stable by the Investigator pending review of the SST as above.

If a subject requires a red blood cell transfusion, then dosing will be suspended until 10 days post-transfusion or until deemed stable (both clinically stable and hemoglobin/red blood cell count is stable) by the Investigator.

Subject will continue scheduled blood draws for safety, PK, and PD during dose suspension for VOC or transfusion.

### Dosing Termination

Further dosing at the current level and dose escalation will be terminated if one of the following is observed:

A single life-threatening SAE, considered to be related to natalizumab by the Investigator and/or Sponsor

Two similar SAEs in subjects receiving natalizumab within the same cohort, unless clearly unrelated to natalizumab (e.g., motor vehicle accident), OR Three or more similar AEs in subjects receiving natalizumab that are either intolerable, as reported by the subject, and/or deemed a medically unacceptable risk by the members of the SST

At the determination of the SST

### Study Termination

This study may be terminated at any time and investigators, study subjects, Investigational Review Boards, Ethics Committees, the US Food and Drug Administration (FDA), and any other applicable regulatory agencies informed. Investigators will be notified if the study is placed on hold, completed, or closed.

End of Study: The end of study is last subject's last visit 3 months after the last infusion (Final Study Visit).

TABLE 3
Schedule of Events - Treatment Phase

| | Pretreatment | Treatment | | | | | | | | | | | |
|---|---|---|---|---|---|---|---|---|---|---|---|---|---|
| | Screening | | Day 1 | Day 2 | Day 4 | Day 8 | Day 15 | Day 22 | Day 29 | Day 36 | Day 43 | Day 50 | Day 57 |
| Tests and | Visit ≦28 | | −4 hrs post | (±6 | (±1 | (±1 | (±3 | (±3 | (±3 | (±3 | (±3 | (±3 | (±3 |
| Assessments | Days^{(a)} | Day 1 | (±1 hr) | hrs) | days) | days) | days) | days) | days) | days) | days) | days) | days) |
| Study Treatment | | X | | | | | | | X | | | | X |
| Administration^{(b)} | | | | | | | | | | | | | |
| Informed Consent | X | | | | | | | | | | | | |
| Demographics, | X | | | | | | | | | | | | |
| Medical History | | | | | | | | | | | | | |
| Inclusion/Exclusion | X | | | | | | | | | | | | |
| Criteria | | | | | | | | | | | | | |
| Randomization | | X | | | | | | | | | | | |
| Physical | X | X | | | | | | | | | | | |
| Examination^{(c)} | | | | | | | | | | | | | |
| Neurologic | X | X | | | | | | | X | | | | X |
| Evaluation^{(d)} | | | | | | | | | | | | | |
| Vital Signs^{(e)} | X | X | X | | | | | | X | | | | X |
| CBC with | X^{(f)} | X | | X | X | X | X | X | X | X | X | X | X |
| Differential and | | | | | | | | | | | | | |
| Absolute | | | | | | | | | | | | | |
| Reticulocyte Count | | | | | | | | | | | | | |
| Chemistry (liver | X | X | | X | X | X | X | X | X | X | X | X | X |
| panel, renal panel, | | | | | | | | | | | | | |
| LDH) | | | | | | | | | | | | | |
| LDH Isoenzymes | X | X | | | | X | X | X | X | X | X | X | X |
| Urinalysis and | X | X | | X | X | X | X | X | X | X | X | X | X |
| Urine Hemoglobin | | | | | | | | | | | | | |
| Virology^{(g)} | X | | | | | | | | | | | | |
| Serum for | X | | | | | | | | | | | | |
| Anti-natalizumab | | | | | | | | | | | | | |
| and Neutralizing | | | | | | | | | | | | | |
| Antibodies^{(h)} | | | | | | | | | | | | | |
| Serum for Anti- | X | | | | | | | | | | | | |
| JCV Antibodies | | | | | | | | | | | | | |
| Immunophenotyping | | X | | X | | X | X | X | X | | | | X |
| (CD34+) | | | | | | | | | | | | | |
| Serum Pregnancy | X | | | | | | | | | | | | |
| Test⁽ⁱ⁾ | | | | | | | | | | | | | |
| Urine Pregnancy | | X | | | | | | | X | | | | X |
| Test⁽ⁱ⁾ | | | | | | | | | | | | | |
| Serum | X | X | X | X | X | X | X | X | X | | | | X |
| Natalizumab | | | | | | | | | | | | | |
| Concentrations^{(h)} | | | | | | | | | | | | | |
| α4 Integrin | X | X | X | X | X | X | X | X | X | | | | X |
| Saturation Assay^{(j)} | | | | | | | | | | | | | |
| VCAM:Ig Flow | X | X | X | X | X | X | X | X | X | | | | X |
| Binding | | | | | | | | | | | | | |
| Plasma for | X | X | | | | | | | | | | | |
| D-dimer, F1.2, and | | | | | | | | | | | | | |
| TAT | | | | | | | | | | | | | |
| Serum for hsCRP | X | X | | | | | | | | | | | |
| Serum for sVCAM | X | X | | | | | | | | | | | |
| Exploratory Serum | X | X | | | | | | | | | | | |
| Biomarkers^{(k)} | | | | | | | | | | | | | |
| Research Assays^{(l)} | X | X | X | | | | | | X | | | | X |
| Optional Genetics | X | | | | | | | | | | | | |
| Sample^{(m)} | | | | | | | | | | | | | |
| Optional RNA | | X | | | | | | | | | | | |
| Sample Collection | | | | | | | | | | | | | |
| Preinfusion | | X | | | | | | | X | | | | X |
| Checklist⁽ⁿ⁾ | | | | | | | | | | | | | |

| | | |
|---|---|---|
| Concomitant | X^{(o)} | Monitor and record throughout study^{(p)} |
| Therapy, | | |
| AEs/SAEs | | |

Abbreviations: AE = adverse event, CBC = complete blood count, HBcAb = hepatitis B core antibody, HBsAg = hepatitis B surface antigen, HCV = hepatitis C virus, HIV = human immunodeficiency virus, hsCRP = high-sensitivity C-reactive protein, IL = interleukin, JCV = John Cunningham virus, LDH = lactate dehydrogenase, PML = progressive multifocal leukoencephalopathy, SCD = sickle cell disease, SAE = serious adverse event, sICAM-1 = soluble intercellular adhesion molecule-1, sVCAM = soluble vascular cell adhesion molecule, TAT = thrombin-antithrombin III complex, TNF-α = tumor necrosis factor-α, TNFR = tumor necrosis factor receptor, VCAM = vascular cell adhesion molecule.
^{(a)}All Screening evaluations must be performed and results reviewed prior to the first study treatment infusion. Screening period may be extended if there are unforeseen delays in receiving laboratory results necessary for assessing eligibility.
^{(b)}All evaluations must be performed prior to study treatment administration at each visit.
^{(c)}Physical examination at Screening Visit includes height and weight measurements, as well as 12 lead electrocardiogram.
^{(d)}Neurological evaluation will include neurologic review of systems, targeted neurologic exam (mental status, cranial nerve, reflex, visual, motor/cerebellar, and sensory evaluations), and identification of signs and symptoms suggestive of PML.
^{(e)}Vital sign measurements include supine diastolic and systolic blood pressure, heart rate, oxygen saturation, respiratory rate, and body temperature. The subject must remain in the same body position quietly for 5 minutes prior to heart rate and blood pressure measurement. When applicable, vital signs evaluation will be performed within 1 hour prior to study drug infusion.
^{(f)}Repeat hemoglobin at Day −10 ± 4 days is allowed for subjects whose initial hemoglobin during Screening is ≧7.5 g/dL but <8 g/dL.
^{(g)}Including virology test for hepatitis C virus (HCV) antibody, HBsAg, HBcAb, and HIV.
^{(h)}Serum sample for natalizumab concentration and anti-natalizumab and neutralizing antibody must be drawn prior to dosing.
⁽ⁱ⁾Required for women of childbearing potential.
^{(j)}Includes assessment of α4 integrin levels on reticulocytes and leukocytes.
^{(k)}Exploratory biomarkers may include, but is not limited to, TNFR-1, IL-8, IL-6, TNF-α, p-selectin, and sICAM-1.
^{(l)}Assays may include, but are not limited to, assays for exploratory research, cell adhesion under flow shear conditions, and cell to cell aggregates.
^{(m)}Sample can be collected at any point during the study.
⁽ⁿ⁾The Investigator will review and approve the preinfusion checklist prior to administering natalizumab.
^{(o)}Only serious pretreatment events and concomitant therapies will be collected between Screening and prior to dosing on Day 1 Visit.
^{(p)}Resolution information on previously reported AEs/SAEs will be collected and the assessment of new AEs/SAEs will be conducted. New or worsening neurologic symptom responses reported on the PML checklist will be followed up with appropriate consultation with a Neurologist and/or brain MRI at the discretion of the Investigator.

TABLE 4
Schedule of Events - Post-treatment Phase

| | | | | | | | | | Final | | |
|---|---|---|---|---|---|---|---|---|---|---|---|
| | Follow-up | | | | | | | | Study | | |
| | Day 57 | | | | | | | | Visit^{(a)} | Early | |
| | −4 hrs | Day | Day | Day | Day | Day | Day | Day | Day | Termi- | Unscheduled |
| Tests and | post | 58 | 60 | 64 | 71 | 78 | 85 | 117 | 147 | nation | SCD |
| Assessments | [symbol] | (±6 hrs) | (±1 day) | (±1 day) | (±3 days) | (±3 days) | (±3 days) | (±7 days) | (±7 days) | Visit^{(b)} | Worsening^{(c)} |
| Study Treatment | | | | | | | | | | | |
| Administration^{(d)} | | | | | | | | | | | |
| Informed Consent | | | | | | | | | | | |
| Demographics, | | | | | | | | | | | |
| Medical History | | | | | | | | | | | |
| Inclusion/Exclusion | | | | | | | | | | | |
| Criteria | | | | | | | | | | | |
| Randomization | | | | | | | | | | | |
| Physical | | | | | | | | | X | X | |
| Examination^{(e)} | | | | | | | | | | | |
| Neurological | | | | | | | | | X | | |
| Evaluation^{(f)} | | | | | | | | | | | |
| Vital Signs^{(g)} | X | | | | | | | X | X | X | X |
| CBC with | | X | X | X | X | X | X | X | X | X | X |
| Differential and | | | | | | | | | | | |
| Absolute | | | | | | | | | | | |
| Reticulocyte Count | | | | | | | | | | | |
| Chemistry (liver | | | | X | X | X | X | X | X | X | X |
| panel, renal panel, | | | | | | | | | | | |
| LDH) | | | | | | | | | | | |
| LDH Isoenzymes | | | | X | X | X | X | X | X | | |
| Urinalysis and Urine | | | | X | X | X | X | X | X | X | |
| Hemoglobin | | | | | | | | | | | |
| Virology^{(h)} | | | | | | | | | | | |
| Serum for | | | | | | | X | | X | X | X |
| Anti-natalizumab | | | | | | | | | | | |
| and Neutralizing | | | | | | | | | | | |
| Antibodies | | | | | | | | | | | |
| Serum for Anti-JCV | | | | | | | | | X^{(j)} | X | |
| Antibodies⁽ⁱ⁾ | | | | | | | | | | | |
| Immunophenotyping | | | | | | | X | X | X | X | X |
| (CD34+) | | | | | | | | | | | |
| Serum Preanancy | | | | | | | | | | | |
| Test^{(k)} | | | | | | | | | | | |
| Urine Pregnancy | | | | | | | | | | | |
| Test^{(k)} | | | | | | | | | | | |
| Serum Natalizumab | X | X | X | X | X | X | X | X | X | X | X |
| Concentrations⁽ⁱ⁾ | | | | | | | | | | | |
| α4 Integrin | X | X | X | X | X | X | X | X | X | X | X |
| Saturation Assay^{(l)} | | | | | | | | | | | |
| VCAM:Ig Flow | X | X | X | X | X | X | X | X | X | X | X |
| Binding | | | | | | | | | | | |
| Plasma for D-dimer, | | | | | | | X | X | X | X | X |
| F1.2, and TAT | | | | | | | | | | | |
| Serum for hsCRP | | | | | | | X | X | X | X | X |
| Serum for sVCAM | | | | | | | X | X | X | X | X |
| Exploratory Serum | | | | | | | X | X | X | X | X |
| Biomarkers^{(m)} | | | | | | | | | | | |
| Research Assays⁽ⁿ⁾ | | | X | X | X | X | X | X | X | X | X |
| Optional Genetics | | | | | | | | | | | |
| Sample^{(o)} | | | | | | | | | | | |
| Optional RNA | | | | | | | X | | | | |
| Sample Collection | | | | | | | | | | | |
| Preinfusion | | | | | | | | | | | |
| Checklist^{(p)} | | | | | | | | | | | |

| | |
|---|---|
| Concomitant | Monitor and record throughout study^{(q)} |
| Therapy, AEs/SAEs | |

Abbreviations: AE = adverse event, CBC = complete blood count, HBcAb = hepatitis B core antibody, HBsAg = hepatitis B surface antigen, HCV = hepatitis C virus, HIV = human immunodeficiency virus, hsCRP = high-sensitivity C-reactive protein, IL = interleukin, JCV = John Cunningham virus, LDH = lactate dehydrogenase, PML = progressive multifocal leukoencephalopathy, SCD = sickle cell disease, SAE = serious adverse event, sICAM-1 = soluble intercellular adhesion molecule-1, sVCAM = soluble vascular cell adhesion molecule, TAT = thrombin-antithrombin III complex, TNF-α = tumor necrosis factor-a, TNFR = tumor necrosis factor receptor, VCAM = vascular cell adhesion molecule.
^{(a)}Subjects who complete the study will return to the site for a Final Study Visit approximately 90 days after their last dose of study treatment.
^{(b)}The Early Termination Visit must be completed within 14 days after the discontinuation of study treatment for subjects who withdraw from the study prematurely. Subjects who terminate early from the study should return to the site for a Follow up Visit approximately 90 days after their last dose of study treatment.
^{(c)}Subjects who suspect that they are experiencing new or worsening SCD symptoms need to contact the investigator via telephone within 24 hours of the onset of symptoms to determine the necessity of an unscheduled visit.
^{(d)}All evaluations must be performed prior to study treatment administration at each visit.
^{(e)}Physical examination at Screening Visit includes height and weight measurements, as well as 12 lead electrocardiogram.
^{(f)}Neurological evaluation will include neurologic review of systems, targeted neurologic exam (mental status, cranial nerve, reflex, visual, motor/cerebellar, and sensory evaluations), and identification of signs and symptoms suggestive of PML.
^{(g)}Vital sign measurements include supine diastolic and systolic blood pressure, heart rate, oxygen saturation, respiratory rate, and body temperature. The subject must remain in the same body position quietly for 5 minutes prior to heart rate and blood pressure measurement. When applicable, vital signs evaluation will be performed within 1 hour prior to study drug infusion.
^{(h)}Including virology test for hepatitis C virus (HCV) antibody, HBsAg, HBcAb, and HIV.
⁽ⁱ⁾Serum sample for natalizumab concentration and anti-natalizumab and neutralizing antibody must be drawn prior to dosing.
^{(j)}This additional anti-natalizumab antibody sample will only be tested if Day 85 sample is positive.
^{(k)}Required for women of childbearing potential.
^{(l)}Includes assessment of α4 integrin levels on reticulocytes and leukocytes.
^{(m)}Exploratory biomarkers may include, but is not limited to, TNFR-1, IL-8, IL-6, TNF-α, p-selectin, and sICAM-1.
⁽ⁿ⁾Assays may include, but are not limited to, assays for exploratory research, cell adhesion under flow shear conditions, and cell to cell aggregates.
^{(o)}Sample can be collected at any point during the study.
^{(p)}The Investigator will review and approve the preinfusion checklist prior to administering natalizumab.
^{(q)}Resolution information on previously reported AEs/SAEs will be collected and the assessment of new AEs/SAEs will be conducted. New or worsening neurologic symptom responses reported on the PML checklist will be followed up with appropriate consultation with a Neurologist and/or brain MRI at the discretion of the Investigator. [symbol]
indicates data missing or illegible when filed

### Example 7

### Use of Hydroxyurea in Combination With Natalizumab

The clinical development plan for natalizumab in SCD is to use natalizumab as a monotherapy or in combination with hydroxyurea. Hydroxyurea has been approved for adults with SCD since 1998, with over 17.5 years of published follow-up data available. Natalizumab can be investigated in patients with SCD with prior or concurrent hydroxyurea use for several reasons outlined below:

1. The mechanism of action of hydroxyurea on the immune system is different than that of immunosuppressants associated with PML risk in MS and CD.

2. Myelosuppression in hydroxyurea-treated patients with SCD can be monitored and is mild, transient, and rapidly reversible.

3. Use of hydroxyurea in SCD is not associated with increased risk of infection.

4. Hydroxyurea has minimal effect on, and may actually enhance, immune function in SCD.

Six cohorts will be enrolled. Subjects within each dose cohort will be continuously enrolled and randomized to receive 3 monthly IV natalizumab or placebo infusions (6:2 ratio) at the following proposed doses:

Cohort A: Natalizumab 150 mg or placebo, HU placebo

Cohort B: Natalizumab 300 mg or placebo, HU placebo

Cohort C: Natalizumab 450 mg or placebo, HU placebo

Cohort D: Natalizumab 150 mg or placebo, HU

Cohort E: Natalizumab 300 mg or placebo, HU

Cohort F: Natalizumab 450 mg or placebo, HU

Hydroxyurea will be administered according to the label. Dosing begins with an initial dose of 15 mg/kg/day in the form of a single dose, with monitoring of the patient's blood count every 2 weeks. If the blood counts are in an acceptable range, the dose may be increased by 5 mg/kg/day every 12 weeks until the MTD of 35 mg/kg/day is reached. If blood counts are between the acceptable range and the toxic range, the dose is not increased. If blood counts are found to be in the toxic range, treatment is discontinued until hematologic recovery. It may then be resumed after the dose is reduced by 2.5 mg/kg/day from the dose associated with hematologic toxicity. The drug may then be titrated up or down every 12 weeks in increments of 2.5 mg/kg/day until the patient is at a stable dose that does not result in hematologic toxicity. Counts considered to be acceptable are: neutrophils greater than or equal to 2500 cells/mm³, platelets greater than or equal to 95,000/mm³, hemoglobin greater than 5.3 g/dl, and reticulocytes greater than or equal to 95,000/ mm³ if the hemoglobin concentration is less than 9 g/dl. Counts considered to be toxic are: neutrophils less than 2000 cells/ mm³, platelets less than 80,000/ mm³, hemoglobin less than 4.5 01, and reticulocytes less than 80,000/ mm³ if the hemoglobin concentration is less than 9 g/dl.

### Example 8

### Natalizumab Blocks Adhesion of Sickle Cell Whole Blood to Activated Endothelial Cells

In the present example, it was investigated whether natalizumab blocks adhesion of whole blood derived from SCD donors to activated endothelial cells under shear dependent flow conditions.

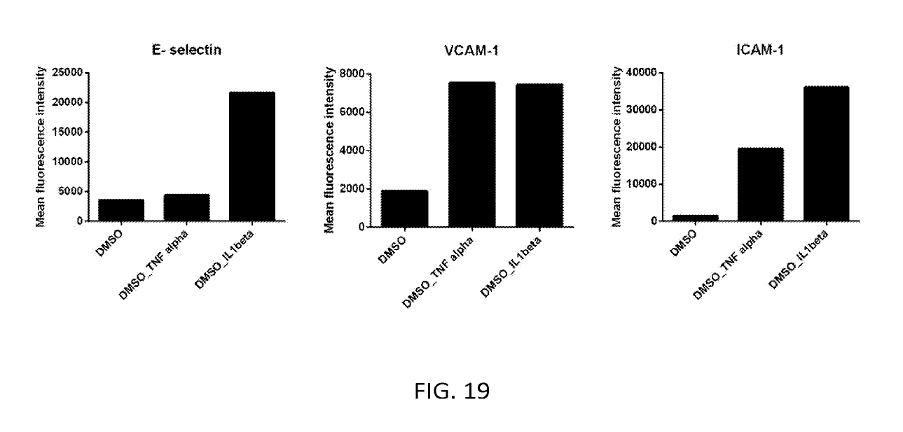

First, the expression of adhesion molecules on cultured human umbilical vein endothelial cells (HUVECs) was characterized. Incubation of HUVECs cultured in gelatin with 10 ng/mL of recombinant human tumor necrosis factor—α (TNF-α) for 18 hours resulted in the upregulation of both Vascular Cell Adhesion Molecule—1 (VCAM-1) and Intercellular Adhesion Molecule-1 (ICAM-1). HUVECs were purchased from Lonza and cultured up to 10 passages. Cells were plated in 10 cm dishes at a density of 1 million cells. After 24 h, cells were incubated with either TNF-α or IL-1β for two time points—6 and 18 hours. Post incubation cells were harvested with EDTA and subjected to flow cytometry to determine surface levels of VCAM-1 and ICAM-1. As shown in FIGS. 18 and 19 IL1-β and TNF-α increase the surface levels of endothelial VCAM-1 and ICAM-1. FIG. 18 depicts adhesion molecule activation (6 hours) and FIG. 19 depicts adhesion molecule activation (18 hours). Both TNF-α and IL1-β activated adhesion molecule surface expression in HUVECs.

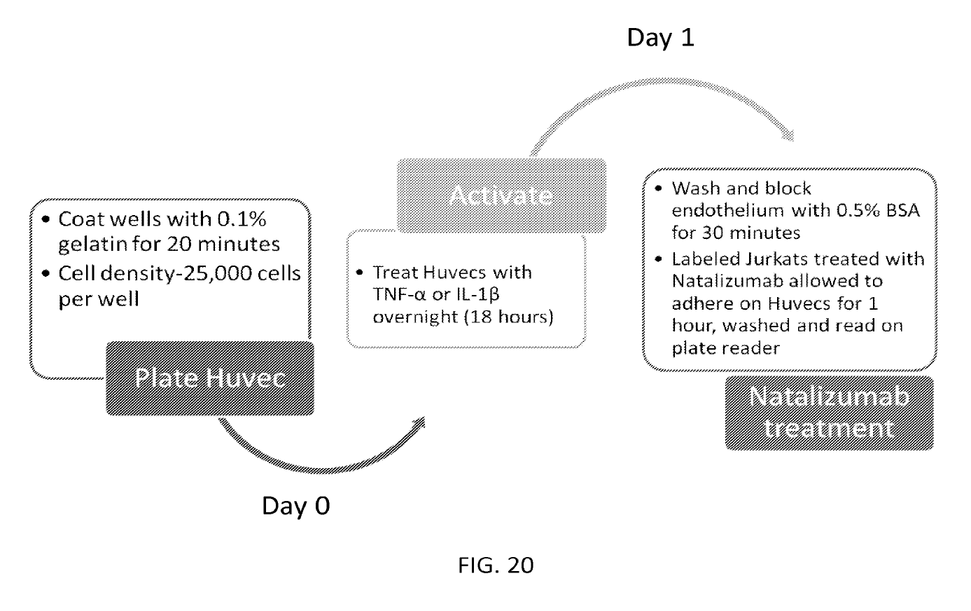

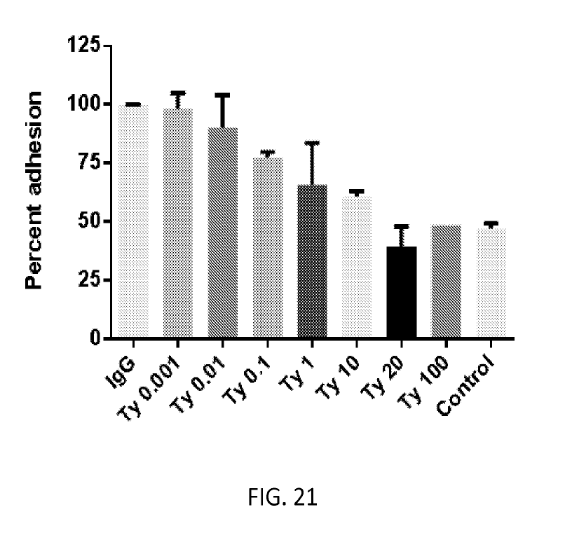

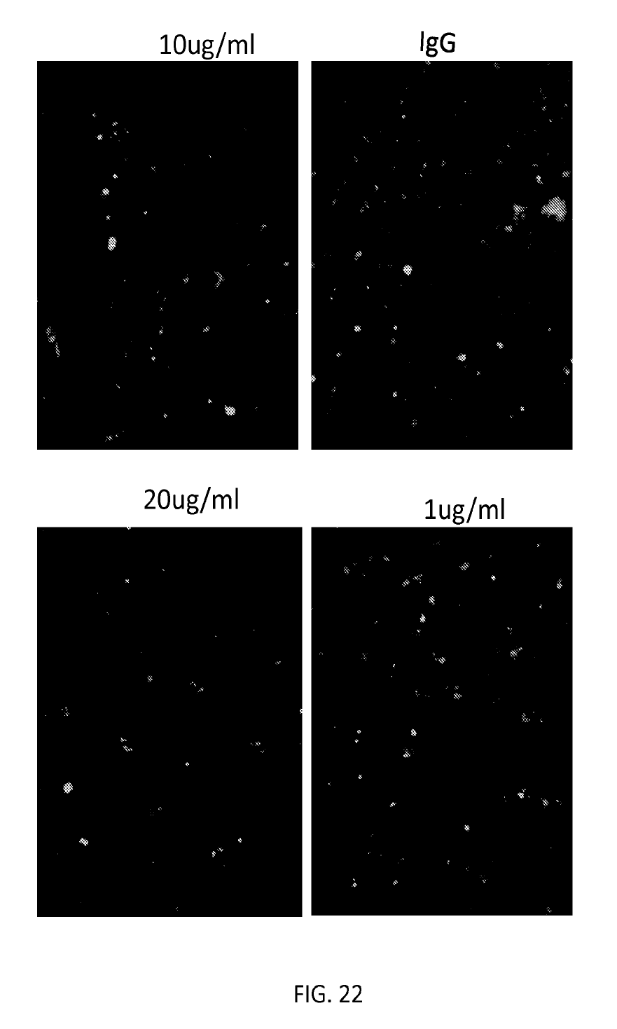

It was further demonstrated that treatment of Jurkat T cells with natalizumab blocked adhesion to TNF-α activated HUVECs in a dose dependent manner in a static endothelial adhesion assay. A workflow for the static adhesion assay is depicted in FIG. 20. HUVECs were activated by 10 ng/ml of TNF-α. As shown in FIG. 21, Jurkat cell adhesion to activated HUVECs was blocked by natalizumab in a dose dependent manner. In a static adhesion assay with reticulocytes, HUVECs were plated and prepared as before. Reticulocytes were isolated from SCD donor whole blood and the following were performed: depletion of CD45+ leukocytes, labeling RBCs with CD71 and isolation of reticulocytes; and labeling reticulocytes with Leukotracker. Reticulocytes were treated with natalizumab and allowed to adhere to HUVECs as for Jurkat cells, and adhesion was quantified by microscopy (FIG. 22). These data demonstrate that natalizumab inhibits Jurkat cell adhesion to HUVECs activated with TNF-α.

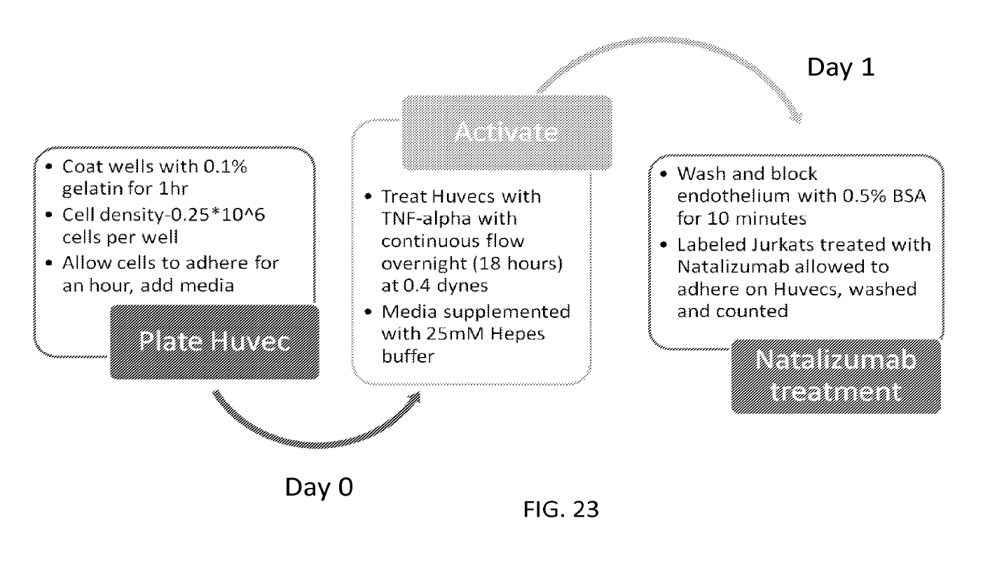

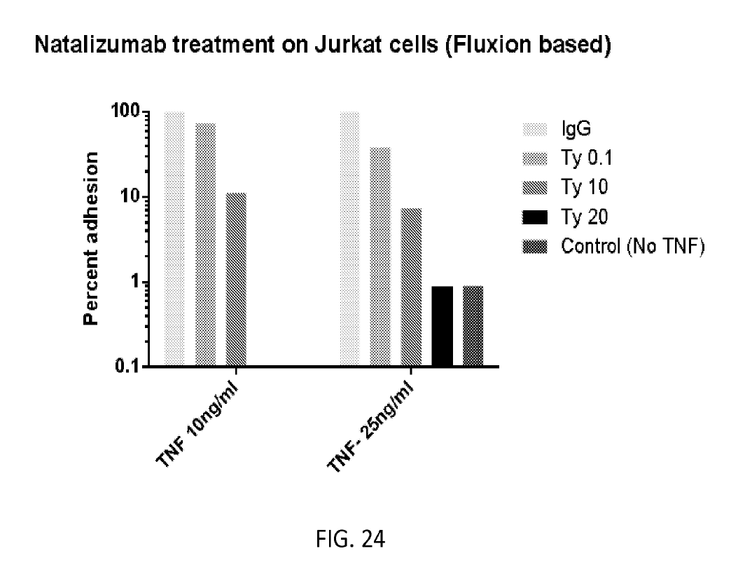

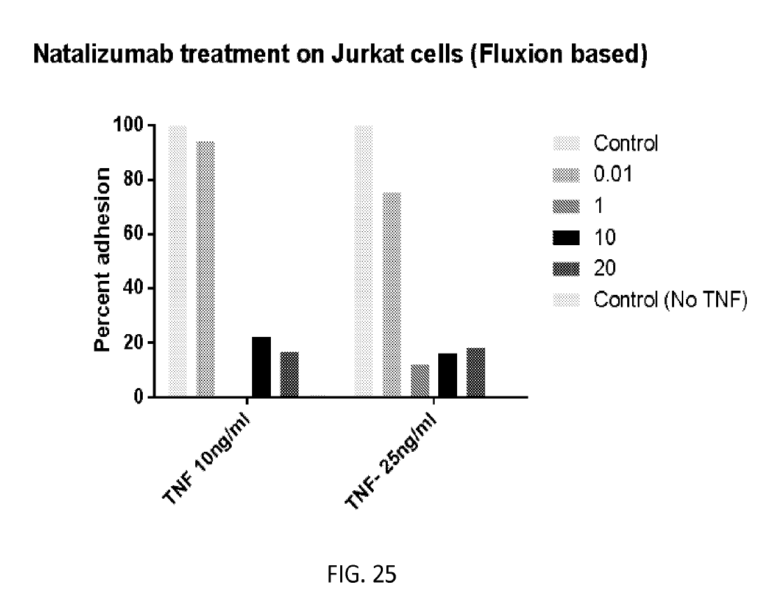

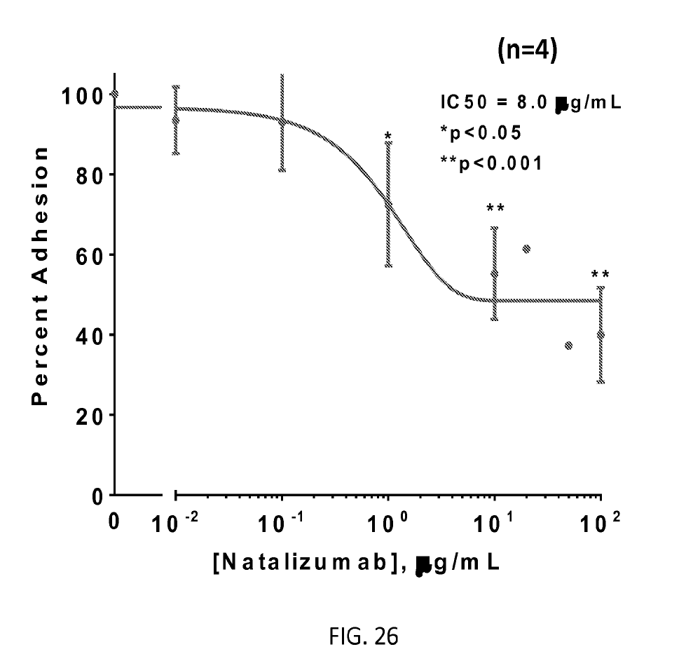

The ability of natalizumab to block adhesion of both Jurkat T-cells and whole blood obtained from sickle cell donors to HUVECs was tested in a microfluidics flow based shear dependent adhesion system. An exemplary workflow for the fluxion based assay is depicted in FIG. 23. Similar to the static adhesion assay, natalizumab showed significant dose dependent inhibition of SCD whole blood cell as well as Jurkat T-cell binding to TNF-α activated HUVECs at a shear dependent flow rate of 1 dyne/cm2. As shown in FIGS. 24 and 25, natalizumab inhibits Jurkat cell adhesion to HUVEC activated with TNF-α. As shown in FIG. 26, natalizumab inhibits SCD whole blood adhesion to HUVECs activated with TNF-α. These results demonstrate that natalizumab is capable of blocking SCD whole blood cells to endothelium and has a potential to be used an anti-adhesive treatment for sickle cell disease.

The present example demonstrates, among other things, that natalizumab inhibits Jurkat cell adhesion to HUVECs under static conditions by 50-60%. Furthermore, endothelial cell plating, culture conditions, and TNF stimulation under flux have been established. VCAM, ICAM, and E-sel staining are in progress. The results presented in the present example demonstrate that, under flux, the inhibition of Jurkat cell adhesion is more potent. Natalizumab also inhibits whole blood adhesion to activated endothelium, and studies are underway using leukocyte depleted blood to assess reticulocyte adhesion.

U.S. Provisional Patent Application Ser. Nos. 62/033,436, filed Aug. 5, 2014 and 62/043,629, filed Aug. 29, 2014 are both incorporated herein in their entirety.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A method of treating a subject suffering from or susceptible to sickle cell disease (SCD), the method comprising: administering to the subject a therapeutically effective amount of a composition comprising a VLA-4 antagonist, wherein the composition is administered such that one or more symptoms of SCD is prevented or reduced.

2. The method of claim 1, wherein the composition is administered such that the number, frequency, and/or duration of vaso-occlusive events in the subject is reduced, e.g., as compared to the number, frequency and/or frequency of vaso-occlusive events in the subject prior to treatment.

3. The method of claim 1 or 2, wherein the composition is administered such that red blood cell survival is increased in the subject, e.g., as compared to the red blood cell survival in the subject prior to treatment.

4. The method of any one of the preceding claims, wherein the composition is administered such that hemoglobin levels are increased in the subject, e.g., as compared to the hemoglobin levels in the subject prior to treatment.

5. The method of any one of the preceding claims, wherein the therapeutically effective amount of the composition is less than 300 mg.

6. The method of any one of the preceding claims, wherein the therapeutically effective amount of the composition is 100-200 mg.

7. The method of claim 6, wherein the therapeutically effective amount of the composition is 150 mg.

8. The method of any one of claims 1-4, wherein the therapeutically effective amount of the composition is 200-400 mg.

9. The method of claim 8, wherein the therapeutically effective amount of the composition is 300 mg.

10. The method of any one of claims 1-4, wherein the therapeutically effective amount of the composition is greater than 300 mg.

11. The method of claim 10, wherein the therapeutically effective amount of the composition is 400-500 mg.

12. The method of claim 11, wherein the therapeutically effective amount of the composition is 450 mg.

13. The method of any one of the preceding claims, wherein the composition is administered by intravenous administration.

14. The method of any one of claims 1-4, wherein the therapeutically effective amount of the composition is 50-100 mg.

15. The method of any one of claims 1-4, wherein the therapeutically effective amount of the composition is 75 mg.

16. The method of claim 14 or 15, wherein the composition is administered subcutaneously.

17. The method of any one of the preceding claims, wherein the VLA-4 antagonist is an anti-VLA-4 antibody molecule, e.g., an anti-VLA-4 antibody molecule described herein.

18. The method of claim 17, wherein the anti- VLA-4 antibody molecule is a monoclonal, a humanized, a human, a chimeric anti-VLA-4 antibody molecule.

19. The method of any one of the preceding claims, wherein the VLA-4 antagonist is an α4-binding fragment of an anti-VLA-4 antibody.

20. The method of claim 19, wherein the α4 binding fragment is an Fab, Fab′, F(ab′)₂, or Fv fragment.

21. The method of any of claims 17-20, wherein the anti- VLA-4 antibody molecule comprises one or more, preferably all, of HC CDR1, HC CDR2, HC CDR3, LC CDR1, LC CDR2 and LC CDR3 of natalizumab.

22. The method of any of claims 17-21, wherein the VLA-4 antagonist is natalizumab.

23. The method of any of the preceeding claims, wherein the VLA-4 antagonist is administered as a monotherapy.

24. The method of any of the preceeding claims, wherein the VLA-4 antagonist is not administered in combination with hydroxyurea.

25. The method of any of claims 1-22, wherein the VLA-4 antagonist is administered in combination with an additional agent or procedure.

26. The method of claim 25, wherein the additional agent is a chemotherapeutic agent.

27. The method of claim 26, wherein the chemotherapeutic agent is hydroxyurea.

28. The method of claim 27, wherein the hydroxyurea is administered to the subject in a dose of between 10 and 40 mg/kg/day.

29. The method of any one of claims 25-28, wherein the VLA-4 antagonist and the additional agent or procedure are administered simultaneously to the subject.

30. The method of any one of claims 25-28, wherein the VLA-4 antagonist and the additional agent or procedure are administered sequentially to the subject.

31. The method of any one of the preceding claims, wherein the subject has not received a previous treatment with a VLA-4 antagonist, e.g., natalizumab.

32. The method of any one of the preceding claims, wherein the subject does not have or is not at risk for developing progressive multifocal leukoencephalopathy (PML).

33. The method of any one of the preceding claims, wherein the subject has greater than 2%, e.g., greater than 5%, e.g., greater than 10%, or more, reticulocytes in their blood.

34. The method of any one of the preceding claims, wherein the subject has 70g/dL or more hemoglobin in their blood before administration.

35. The method of any one of the preceding claims, wherein the subject has 80g/dL or more hemoglobin in their blood before administration.

36. The method of any one of the preceding claims, administration is temporarily discontinued if the subject has less than 67 g/dL hemoglobin in their blood.

37. The method of any one of the preceding claims, administration is permanently discontinued if the subject has less than 55 g/dL hemoglobin in their blood.

38. The method of any one of the preceding claims, administration is permanently if hemoglobin levels in the subject's blood decrease by more than 25 g/L over a 1 week period.

39. A method of treating an acute vaso-occlusive event in a subject, the method comprising: administering to the subject a therapeutically effective amount of a composition comprising a VLA-4 antagonist, wherein the composition is administered such that the severity and/or frequency of the acute vaso-occlusive event is reduced in the subject.

40. The method of claim 39, wherein the composition is administered to the subject within 7 days, 6 days, 5 days, 4 days, 3 days, 2 days, or 1 day after the onset of the vaso-occlusive event in the subject.

41. The method of claim 39 or 40, wherein the therapeutically effective amount of the composition is less than 300 mg.

42. The method of any one of claims 39-41, wherein the therapeutically effective amount of the composition is 100-200 mg.

43. The method of claim 42, wherein the therapeutically effective amount of the composition is 150 mg.

44. The method of claim 39 or 40, wherein the therapeutically effective amount of the composition is 200-400 mg.

45. The method of claim 44, wherein the therapeutically effective amount of the composition is 300 mg.

46. The method of any one of claim 39 or 40, wherein the therapeutically effective amount of the composition is greater than 300 mg.

47. The method of claim 39 or 40, wherein the therapeutically effective amount of the composition is 400-500 mg.

48. The method of claim 47, wherein the therapeutically effective amount of the composition is 450 mg.

49. The method of any one of claims 39-48, wherein the composition is administered by intravenous administration.

50. The method of claim 39 or 40, wherein the therapeutically effective amount of the composition is 50-100 mg.

51. The method of claim 50, wherein the therapeutically effective amount of the composition is 75 mg.

52. The method of claim 50 or 51, wherein the composition is administered subcutaneously.

53. The method of any one of claims 39-52, wherein the VLA-4 antagonist is an anti-VLA-4 antibody molecule, e.g., an anti-VLA-4 antibody molecule described herein.

54. The method of claim 53, wherein the anti- VLA-4 antibody molecule is a monoclonal, a humanized, a human, a chimeric anti-VLA-4 antibody molecule.

55. The method of any one of claims 39-52, wherein the VLA-4 antagonist is an α4-binding fragment of an anti-VLA-4 antibody.

56. The method of claim 55, wherein the α4 binding fragment is an Fab, Fab′, F(ab′)2, or Fv fragment.

57. The method of any of claims 53-56, wherein the anti-VLA-4 antibody molecule comprises one or more, preferably all, of HC CDR1, HC CDR2, HC CDR3, LC CDR1, LC CDR2 and LC CDR3 of natalizumab.

58. The method of any of claims 39-57, wherein the VLA-4 antagonist is natalizumab.

59. The method of any of claims 39-58, wherein the VLA-4 antagonist is administered as a monotherapy.

60. The method of any of claims 39-58, wherein the VLA-4 antagonist is not administered in combination with hydroxyurea.

61. The method of any one of claims 39-58, wherein the VLA-4 antagonist is administered in combination with an additional agent or procedure.

62. The method of claim 61, wherein the additional agent is a chemotherapeutic agent.

63. The method of claim 62, wherein the chemotherapeutic agent is hydroxyurea.

64. The method of claim 63, wherein the hydroxyurea is administered to the subject in a dose of 10 to 40 mg/kg/day.

65. The method of claim 61, wherein the additional agent is an analgesic, e.g., an opiod analgesic.

66. The method of claim 61, wherein the additional procedure is a transfusion, e.g., a red blood cell transfusion or a transplant, e.g., a hematopoietic stem cell transplant (HSCT).

67. The method of any one of claims 39-66, wherein the VLA-4 antagonist and the additional agent or procedure are administered simultaneously to the subject.

68. The method of any one of claims 39-66, wherein the VLA-4 antagonist and the additional agent or procedure are administered sequentially to the subject.

69. The method of any one of claims 39-68, wherein the subject has not received a previous treatment with a VLA-4 antagonist, e.g., natalizumab.

70. The method of any one of claims 39-69, wherein the subject does not have or is not at risk for developing progressive multifocal leukoencephalopathy (PML).

71. The method of any one of the preceding claims, wherein the subject is an adult human subject.

72. The method of any one of the preceding claims, wherein the subject is a pediatric human subject, e.g., 18 years or younger.

73. The method of any one of claims 39-72, wherein the subject has greater than 2%, e.g., greater than 5%, e.g., greater than 10%, or more, reticulocytes in their blood.

74. The method of any one of claims 39-73, wherein the subject has 70 g/dL or more hemoglobin in their blood before administration.

75. The method of any one of claims 39-74, wherein the subject has 80 g/dL or more hemoglobin in their blood before administration.

76. The method of any one of claims 39-75, administration is temporarily discontinued if the subject has less than 67 g/dL hemoglobin in their blood.

77. The method of any one of claims 39-76, administration is permanently discontinued if the subject has less than 55 g/dL hemoglobin in their blood.

78. The method of any one of claims 39-77, administration is permanently if hemoglobin levels in the subject's blood decrease by more than 25 g/L over a 1 week period.

79. A method of evaluating a sample comprising blood cells from a subject, the method comprising:
(a) subjecting a first sample comprising blood cells that has been isolated from the subject to a flow adhesion assay through a channel, e.g., by perfusion via one or more microfluidic channels, wherein the channel is coated with VCAM-land wherein the flow adhesion assay is performed under shear stress conditions;
(b) determining a level of adhesion of blood cells from the first sample to the channel;
(c) contacting a second sample comprising blood cells that has been isolated from the subject with a VLA-4 antagonist, e.g., a VLA-4 binding antibody described herein;
(d) subjecting the second sample to flow adhesion assay through a channel, e.g., by perfusion via one or more microfluidic channels, wherein the channel is coated with VCAM-land wherein the flow adhesion assay is performed under shear stress conditions;
(e) determining a level of adhesion of blood cells from the second sample to the channel, e.g., microfluidic channel; and
(f) identifying the subject as a candidate for treatment with a VLA-4 antagonist, e.g., a VLA-4 binding antibody described herein, if the level of adhesion determined in (e) is less than the level of adhesion determined in (b).

80. The method of claim 79, further comprising a step of obtaining the sample comprising blood cells from the subject.

81. The method of claim 79 or 80, further comprising a step of administering a VLA-4 antagonist, e.g., a VLA-4 binding antibody described herein, to the subject.

82. The method of any one of claims 79-81, wherein the blood cells are red blood cells (RBCs).

83. The method of any one of claims 79-82, wherein the blood cells are reticulocytes.

84. The method of any of claims 1-78, wherein the subject is selected for treatment with the VLA-4 antagonist based upon an evaluation of any of claims 79-83.

85. A method of reducing the frequency of an acute vaso-occlusive event in a subject, the method comprising: administering to the subject a therapeutically effective amount of a composition comprising a VLA-4 antagonist, wherein the composition is administered such that the frequency of the acute vaso-occlusive event is reduced in the subject.

86. The method of claim 85, wherein the therapeutically effective amount of the composition is less than 300 mg.

87. The method of any one of claim 85 or 86, wherein the therapeutically effective amount of the composition is 100-200 mg.

88. The method of claim 87, wherein the therapeutically effective amount of the composition is 150 mg.

89. The method of claim 85, wherein the therapeutically effective amount of the composition is 200-400 mg.

90. The method of claim 89, wherein the therapeutically effective amount of the composition is 300 mg.

91. The method of any one of claims 85, wherein the therapeutically effective amount of the composition is greater than 300 mg.

92. The method of claim 85, wherein the therapeutically effective amount of the composition is 400-500 mg.

93. The method of claim 92, wherein the therapeutically effective amount of the composition is 450 mg.

94. The method of any one of claims 85-93, wherein the composition is administered by intravenous administration.

95. The method of claim 85, wherein the therapeutically effective amount of the composition is 50-100 mg.

96. The method of claim 95, wherein the therapeutically effective amount of the composition is 75 mg.

97. The method of claim 95 or 96, wherein the composition is administered subcutaneously.

98. The method of any one of claims 85-97, wherein the VLA-4 antagonist is an anti-VLA-4 antibody molecule, e.g., an anti-VLA-4 antibody molecule described herein.

99. The method of claim 98, wherein the anti- VLA-4 antibody molecule is a monoclonal, a humanized, a human, a chimeric anti-VLA-4 antibody molecule.

100. The method of any one of claims 85-97, wherein the VLA-4 antagonist is an α4-binding fragment of an anti-VLA-4 antibody.

101. The method of claim 100, wherein the α4 binding fragment is an Fab, Fab′, F(ab′)2, or Fv fragment.

102. The method of any of claims 98-101, wherein the anti-VLA-4 antibody molecule comprises one or more, preferably all, of HC CDR1, HC CDR2, HC CDR3, LC CDR1, LC CDR2 and LC CDR3 of natalizumab.

103. The method of any of claims 85-102, wherein the VLA-4 antagonist is natalizumab.

104. The method of any of claims 85-103, wherein the VLA-4 antagonist is administered as a monotherapy.

105. The method of any of claims 85-103, wherein the VLA-4 antagonist is not administered in combination with hydroxyurea.

106. The method of any one of claims 85-103, wherein the VLA-4 antagonist is administered in combination with an additional agent or procedure.

107. The method of claim 106, wherein the additional agent is a chemotherapeutic agent.

108. The method of claim 107, wherein the chemotherapeutic agent is hydroxyurea.

109. The method of claim 108, wherein the hydroxyurea is administered to the subject in a dose of 10 to 40 mg/kg/day.

110. The method of claim 106, wherein the additional agent is an analgesic, e.g., an opiod analgesic.

111. The method of claim 106, wherein the additional procedure is a transfusion, e.g., a red blood cell transfusion or a transplant, e.g., a hematopoietic stem cell transplant (HSCT).

112. The method of any one of claims 85-106, wherein the VLA-4 antagonist and the additional agent or procedure are administered simultaneously to the subject.

113. The method of any one of claims 85-106, wherein the VLA-4 antagonist and the additional agent or procedure are administered sequentially to the subject.

114. The method of any one of claims 85-113, wherein the subject has not received a previous treatment with a VLA-4 antagonist, e.g., natalizumab.

115. The method of any one of claims 85-114, wherein the subject does not have or is not at risk for developing progressive multifocal leukoencephalopathy (PML).

116. The method of any one of claims 85-115, wherein the subject is an adult human subject.

117. The method of any one of claims 85-116, wherein the subject is a pediatric human subject, e.g., 18 years or younger.

118. The method of any one of claims 85-117, wherein the subject has greater than 2%, e.g., greater than 5%, e.g., greater than 10%, or more, reticulocytes in their blood.

119. The method of any one of claims 85-118, wherein the subject has 70 g/dL or more hemoglobin in their blood before administration.

120. The method of any one of claims 85-119, wherein the subject has 80 g/dL or more hemoglobin in their blood before administration.

121. The method of any one of claims 85-120, administration is temporarily discontinued if the subject has less than 67 g/dL hemoglobin in their blood.

122. The method of any one of claims 85-121, administration is permanently discontinued if the subject has less than 55 g/dL hemoglobin in their blood.

123. The method of any one of claims 85-122, administration is permanently if hemoglobin levels in the subject's blood decrease by more than 25 g/L over a 1 week period.
